(19) United States
(12) Patent Application Publication
(10) Pub. No.: US 2021/0147865 A1
(43) Pub. Date: May 20, 2021

(54) INCREASING SALT TOLERANCE IN PLANTS

(71) Applicant: ARIZONA BOARD OF REGENTS ON BEHALF OF THE UNIVERSITY OF ARIZONA, Tucson, AZ (US)

(72) Inventors: Karen SCHUMAKER, Tucson, AZ (US); Shea MONIHAN, Tucson, AZ (US); Choong-Hwan RYU, Tucson, AZ (US)

(73) Assignee: ARIZONA BOARD OF REGENTS ON BEHALF OF THE UNIVERSITY OF ARIZONA, Tucson, AZ (US)

(21) Appl. No.: 17/047,539

(22) PCT Filed: Apr. 19, 2019
(86) PCT No.: PCT/US2019/028346

§ 371 Date: Oct. 14, 2020

Related U.S. Application Data
(60) Provisional application No. 62/660,757, filed on Apr. 20, 2018.

(51) Int. Cl. C12N 15/82

(52) U.S. Cl. CPC C12N 15/8273

(57) ABSTRACT

The present technology provides a duplicated Eutrema salsugineum CALCINEURIN B-LIKE 10 (CBL10) gene (EsCBL10a and EsCBL10b), nucleic acids that encode the EsCBL10a and EsCBL10b proteins, and methods for increasing salt tolerance in a plant by genetically engineering the plant to express EsCBL10a and/or EsCBL10b protein. Also provided are transgenic plants expressing the EsCBL10a and/or EsCBL10b protein and having increased salt tolerance.

### CROSS-REFERENCE TO RELATED APPLICATIONS

This application claims the benefit of and priority to U.S. Provisional Patent Application No. 62/660,757, filed Apr. 20, 2018, the entire contents of which are incorporated herein by reference.

### GOVERNMENT SUPPORT

This invention was made with government support under Grant No. 1552099, awarded by NSF. The government has certain rights in the invention.

### TECHNICAL FIELD

The present technology relates generally to a duplicated Eutrema salsugineum CALCINEURIN B-LIKE 10 (CBL10) gene (EsCBL10a and EsCBL10b), nucleic acids that encode the EsCBL10a and EsCBL10b proteins, and methods for increasing salt tolerance in a plant by genetically engineering the plant to express EsCBL10a and/or EsCBL10b protein. The present technology also relates to transgenic plants expressing the EsCBL10a and/or EsCBL10b protein.

### BACKGROUND

The following description is provided to assist the understanding of the reader. None of the information provided or references cited is admitted to be prior art.

A growing challenge to food security is the degradation of arable land due to the accumulation of salt in the soil. Current climate prediction models indicate that average surface temperatures will rise by 3-5° C. in the next 50-100 years, drastically affecting worldwide agricultural systems. Increasing temperatures are expected to result in warmer, drier summers, with a reduction in growing seasons in many regions and increased salinization resulting in decreased amounts of land suitable for agriculture. The estimated annual global cost of crop loss due to salt-affected soils is $27.3 billion (Qadir, et al., 2014). The Food and Agricultural Organization estimates that 20% of irrigated land, which produces over 40% of the world's food, is affected by salt, and 1-2% of irrigated land is lost each year due to the accumulation of salt (FAO, 2002). The changing climatic conditions, coupled with increasing pressure on global food productivity due to the demands of a growing population, underscore the importance of developing methods to enhance abiotic stress tolerance in plants, such as increased salt tolerance, leading to improved crop productivity.

Accordingly, there is a need to develop methods for increasing salt tolerance in plants, particularly those that are useful as agricultural crops. Practical solutions for sustainable agriculture will require integrated approaches including identification of genes and mechanisms that contribute to salt tolerance for natural trait variation and transgenic approaches.

### SUMMARY

In one aspect, the present disclosure provides an isolated cDNA molecule comprising a nucleotide sequence selected from the group consisting of: (a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24; (b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25; (c) a nucleotide sequence that is at least 90% identical to the nucleotide sequence of (a) or (b); (d) a nucleotide sequence that differs from the nucleotide sequence of (a) or (b) due to degeneracy of the genetic code; and (e) any combination of the nucleotide sequences of (a)-(d). In some embodiments, the nucleotide sequence is operably linked to a heterologous nucleic acid. In some embodiments, the heterologous nucleic acid is a promoter.

In some embodiments, the present disclosure provides an expression vector comprising an isolated cDNA molecule comprising a nucleotide sequence selected from the group consisting of: (a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24; (b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25; (c) a nucleotide sequence that is at least 90% identical to the nucleotide sequence of (a) or (b); (d) a nucleotide sequence that differs from the nucleotide sequence of (a) or (b) due to degeneracy of the genetic code; and (e) any combination of the nucleotide sequences of (a)-(d), operable linked to one or more control sequences suitable for directing expression in a host cell.

In some embodiments, the present disclosure provides a transgenic plant comprising a cell comprising a chimeric nucleic acid construct comprising an isolated cDNA molecule comprising a nucleotide sequence selected from the group consisting of: (a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24; (b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25; (c) a nucleotide sequence that is at least 90% identical to the nucleotide sequence of (a) or (b); (d) a nucleotide sequence that differs from the nucleotide sequence of (a) or (b) due to degeneracy of the genetic code; and (e) any combination of the nucleotide sequences of (a)-(d). In some embodiments, the transgenic plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny plant thereof, wherein the seeds comprise the chimeric nucleic acid construct. In some embodiments, the present disclosure provides a crop comprising a plurality of transgenic plants.

In some embodiments, the nucleotide sequence is set forth in SEQ ID NO: 1.

In some embodiments, the nucleotide sequence is set forth in SEQ ID NO: 2.

In some embodiments, the nucleotide sequence is set forth in SEQ ID NO: 4.

In some embodiments, the nucleotide sequence is set forth in SEQ ID NO: 5.

In some embodiments, the nucleotide sequence is set forth in SEQ ID NO: 10.

In some embodiments, the nucleotide sequence is set forth in SEQ ID NO: 12.

In some embodiments, the nucleotide sequence is set forth in SEQ ID NO: 14.

In some embodiments, the nucleotide sequence is set forth in SEQ ID NO: 16.

In some embodiments, the nucleotide sequence is set forth in SEQ ID NO: 18.

In some embodiments, the nucleotide sequence is set forth in SEQ ID NO: 20.

In some embodiments, the nucleotide sequence is set forth in SEQ ID NO: 22.

In some embodiments, the nucleotide sequence is set forth in SEQ ID NO: 24.

In one aspect, the present disclosure provides a method for increasing salt tolerance in a plant in need thereof, comprising: growing the plant under conditions which allow for the expression of a Eutrema salsugineum CALCINEURIN B-LIKE10 (EsCBL10) gene product from an exogenous expression vector in the plant, the vector comprising a promoter that is functional in a plant cell operably linked to a nucleotide sequence selected from the group consisting of: (a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24; (b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25; (c) a nucleotide sequence that is at least 90% identical to the nucleotide sequence of (a) or (b), and which encodes an EsCBL10 protein; (d) a nucleotide sequence that differs from the nucleotide sequence of (a) or (b) due to degeneracy of the genetic code; and (e) any combination of the nucleotide sequences of (a)-(d); and wherein expression of the EsCBL10 gene product results in the plant having an increased salt tolerance as compared to a control plant grown under similar conditions.

In some embodiments, the vector comprises the nucleotide sequence set forth in SEQ ID NO: 2.

In some embodiments, the vector comprises the nucleotide sequence set forth in SEQ ID NO: 5.

In some embodiments, the vector comprises the nucleotide sequence set forth in SEQ ID NO: 10.

In some embodiments, the vector comprises the nucleotide sequence set forth in SEQ ID NO: 12.

In some embodiments, the vector comprises the nucleotide sequence set forth in SEQ ID NO: 14.

In some embodiments, the vector comprises the nucleotide sequence set forth in SEQ ID NO: 16.

In some embodiments, the vector comprises the nucleotide sequence set forth in SEQ ID NO: 18.

In some embodiments, the vector comprises the nucleotide sequence set forth in SEQ ID NO: 20.

In some embodiments, the vector comprises the nucleotide sequence set forth in SEQ ID NO: 22.

In some embodiments, the vector comprises the nucleotide sequence set forth in SEQ ID NO: 24.

In some embodiments, the increased salt tolerance comprises an improvement in fresh weight, root length, or any combination thereof, as compared to a control plant grown under similar conditions.

In some embodiments, the present disclosure provides a transgenic plant produced by a method comprising growing the plant under conditions which allow for the expression of a Eutrema salsugineum CALCINEURIN B-LIKE10 (EsCBL10) gene product from an exogenous expression vector in the plant, the vector comprising a promoter that is functional in a plant cell operably linked to a nucleotide sequence selected from the group consisting of: (a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24; (b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25; (c) a nucleotide sequence that is at least 90% identical to the nucleotide sequence of (a) or (b), and which encodes an EsCBL10 protein; (d) a nucleotide sequence that differs from the nucleotide sequence of (a) or (b) due to degeneracy of the genetic code; and (e) any combination of the nucleotide sequences of (a)-(d); wherein the plant comprises the exogenous expression vector expressing the EsCBL10 gene product and has increased salt tolerance as compared to a control plant grown under similar conditions.

In some embodiments, the transgenic plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny thereof.

In one aspect, the present disclosure provides a method for increasing salt tolerance in a plant in need thereof, comprising introducing into the plant: (i) a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); and (ii) a second vector comprising a nucleotide sequence selected from the group consisting of: (f) the nucleotide sequence set forth in SEQ ID NO: 4 or SEQ ID NO: 5; (g) a nucleotide sequence that encodes a polypeptide having the amino acid sequence set forth in SEQ ID NO: 6; (h) a nucleotide sequence that is at least 90% identical to the nucleotide sequence of (f) or (g), and which encodes an EsCBL10b protein; (i) a nucleotide sequence that differs from the nucleotide sequence of (f) or (g) due to degeneracy of the genetic code; and (j) any combination of the nucleotide sequences of (f)-(i); growing the plant under conditions which allow for the expression of an EsCBL10a and EsCBL10b gene product from the nucleotide sequence; wherein expression of the EsCBL10a and EsCBL10b gene products results in the plant having an increased salt tolerance as compared to a control plant grown under similar conditions.

In some embodiments, the first vector comprises the nucleotide sequence set forth in SEQ ID NO: 2 and the second vector comprises the nucleotide sequence set forth in SEQ ID NO: 5.

In some embodiments, the increased salt tolerance comprises an improvement in fresh weight, root length, or any combination thereof, as compared to a control plant grown under similar conditions.

In some embodiments, the present disclosure provides a transgenic plant produced by a method comprising introducing into the plant: (i) a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); and (ii) a second vector comprising a nucleotide sequence selected from the group consisting of: (f) the nucleotide sequence set forth in SEQ ID NO: 4 or SEQ ID NO: 5; (g) a nucleotide sequence that encodes a polypeptide having the amino acid sequence set forth in SEQ ID NO: 6; (h) a nucleotide sequence that is at least 90% identical to the nucleotide sequence of (f) or (g), and which encodes an EsCBL10b protein; (i) a nucleotide sequence that differs from the nucleotide sequence of (f) or (g) due to degeneracy of the genetic code; and (j) any combination of the nucleotide sequences of (f)-(i); growing the plant under conditions which allow for the expression of an EsCBL10a and EsCBL10b gene product from the nucleotide sequence; wherein the plant expresses an EsCBL10a and EsCBL10b gene product and has increased salt tolerance as compared to a control plant grown under similar conditions.

In some embodiments, the transgenic plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny thereof.

In one aspect, the present disclosure provides a method for increasing salt tolerance in a plant in need thereof, comprising introducing into the plant: (i) a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); and (ii) a second vector comprising a nucleotide sequence selected from the group consisting of: (f) the nucleotide sequence set forth in SEQ ID NO: 7 or SEQ ID NO: 8; (g) a nucleotide sequence that encodes a polypeptide having the amino acid sequence set forth in SEQ ID NO: 9; (h) a nucleotide sequence that is at least 90% identical to the nucleotide sequence of (f) or (g), and which encodes a EsSOS3 protein; (i) a nucleotide sequence that differs from the nucleotide sequence of (f) or (g) due to degeneracy of the genetic code; and (j) any combination of the nucleotide sequences of (f)-(i); growing the plant under conditions which allow for the expression of an EsCBL10a and EsSOS3 gene product from the nucleotide sequence; wherein expression of the EsCBL10a and EsSOS3 gene products results in the plant having an increased salt tolerance as compared to a control plant grown under similar conditions.

In some embodiments, the first vector comprises the nucleotide sequence set forth in SEQ ID NO: 2 and the second vector comprises the nucleotide sequence set forth in SEQ ID NO: 8.

In some embodiments, the increased salt tolerance comprises an improvement in fresh weight, root length, or any combination thereof, as compared to a control plant grown under similar conditions.

In some embodiments, the present disclosure provides a transgenic plant produced by a method comprising introducing into the plant: (i) a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); and (ii) a second vector comprising a nucleotide sequence selected from the group consisting of: (f) the nucleotide sequence set forth in SEQ ID NO: 7 or SEQ ID NO: 8; (g) a nucleotide sequence that encodes a polypeptide having the amino acid sequence set forth in SEQ ID NO: 9; (h) a nucleotide sequence that is at least 90% identical to the nucleotide sequence of (f) or (g), and which encodes a EsSOS3 protein; (i) a nucleotide sequence that differs from the nucleotide sequence of (f) or (g) due to degeneracy of the genetic code; and (j) any combination of the nucleotide sequences of (f)-(i); growing the plant under conditions which allow for the expression of an EsCBL10a and EsSOS3 gene product from the nucleotide sequence; wherein the plant expresses an EsCBL10a and EsSOS3 gene product and has increased salt tolerance as compared to a control plant grown under similar conditions.

In some embodiments, the transgenic is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny thereof.

In one aspect, the present disclosure provides a method for increasing salt tolerance in a plant comprising: crossing a first plant comprising a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); with a second plant comprising a second vector comprising a nucleotide sequence selected from the group consisting of: (f) the nucleotide sequence set forth in SEQ ID NO: 4 or SEQ ID NO: 5; (g) a nucleotide sequence that encodes a polypeptide having the amino acid sequence set forth in SEQ ID NO: 6; (h) a nucleotide sequence that is at least 90% identical to the nucleotide sequence of (f) or (g), and which encodes an EsCBL10b protein; (i) a nucleotide sequence that differs from the nucleotide sequence of (f) or (g) due to degeneracy of the genetic code; and (j) any combination of the nucleotide sequences of (f)-(i); to produce a plant expressing EsCBL10a and EsCBL10b gene product and having an increased salt tolerance as compared to a control plant.

In some embodiments, the first vector comprises the nucleotide sequence set forth in SEQ ID NO: 2 and the second vector comprises the nucleotide sequence set forth in SEQ ID NO: 5.

In some embodiments, the increased salt tolerance comprises an improvement in fresh weight, root length, or any combination thereof, as compared to a control plant grown under similar conditions.

In some embodiments, the present disclosure provides a transgenic plant produced by a method comprising: crossing a first plant comprising a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); with a second plant comprising a second vector comprising a nucleotide sequence selected from the group consisting of: (f) the nucleotide sequence set forth in SEQ ID NO: 4 or SEQ ID NO: 5; (g) a nucleotide sequence that encodes a polypeptide having the amino acid sequence set forth in SEQ ID NO: 6; (h) a nucleotide sequence that is at least 90% identical to the nucleotide sequence of (f) or (g), and which encodes an EsCBL10b protein; (i) a nucleotide sequence that differs from the nucleotide sequence of (f) or (g) due to degeneracy of the genetic code; and (j) any combination of the nucleotide sequences of (f)-(i); wherein the plant expresses an EsCBL10a and EsCBL10b gene product and has increased salt tolerance as compared to a control plant.

In some embodiments, the transgenic plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny thereof.

In one aspect, the present disclosure provides a method for increasing salt tolerance in a plant comprising: crossing a first plant comprising a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); with a second plant comprising a second vector comprising a nucleotide sequence selected from the group consisting of: (f) the nucleotide sequence set forth in SEQ ID NO: 7 or SEQ ID NO: 8; (g) a nucleotide sequence that encodes a polypeptide having the amino acid sequence set forth in SEQ ID NO: 9; (h) a nucleotide sequence that is at least 90% identical to the nucleotide sequence of (f) or (g), and which encodes a EsSOS3 protein; (i) a nucleotide sequence that differs from the nucleotide sequence of (f) or (g) due to degeneracy of the genetic code; and (j) any combination of the nucleotide sequences of (f)-(i); to produce a plant expressing EsCBL10a and EsSOS3 gene product and having an increased salt tolerance as compared to a control plant.

In some embodiments, the first vector comprises the nucleotide sequence set forth in SEQ ID NO: 2 and the second vector comprises the nucleotide sequence set forth in SEQ ID NO: 8.

In some embodiments, the increased salt tolerance comprises an improvement in fresh weight, root length, or any combination thereof, as compared to a control plant grown under similar conditions.

In some embodiments, the present disclosure provides a transgenic plant produced by a method comprising: crossing a first plant comprising a first vector comprising a nucleotide sequence selected from the group consisting of: (a) the nucleotide sequence set forth in SEQ ID NO: 1 or SEQ ID NO: 2; (b) a nucleotide sequence that encodes a polypeptide having the amino acid sequence set forth in SEQ ID NO: 3; (c) a nucleotide sequence that is at least 90% identical to the nucleotide sequence of (a) or (b), and which encodes an EsCBL10a protein; (d) a nucleotide sequence that differs from the nucleotide sequence of (a) or (b) due to degeneracy of the genetic code; and (e) any combination of the nucleotide sequences of (a)-(d); with a second plant comprising a second vector comprising a nucleotide sequence selected from the group consisting of: (f) the nucleotide sequence set forth in SEQ ID NO: 7 or SEQ ID NO: 8; (g) a nucleotide sequence that encodes a polypeptide having the amino acid sequence set forth in SEQ ID NO: 9; (h) a nucleotide sequence that is at least 90% identical to the nucleotide sequence of (f) or (g), and which encodes a EsSOS3 protein; (i) a nucleotide sequence that differs from the nucleotide sequence of (f) or (g) due to degeneracy of the genetic code; and (j) any combination of the nucleotide sequences of (f)-(i); wherein the plant expresses an EsCBL10a and EsSOS3 gene product and has increased salt tolerance as compared to a control plant.

In some embodiments, the transgenic plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

In some embodiments, the present disclosure provides seeds from the transgenic plant or a progeny thereof.

The technology described and claimed herein has many attributes and embodiments including, but not limited to, those set forth or described or referenced in this brief summary. It is not intended to be all-inclusive and the technology described and claimed herein is not limited to or by the features or embodiments identified in this brief summary, which is included for purposes of illustration only and not restriction. Additional embodiments may be disclosed in the detailed description below.

### BRIEF DESCRIPTION OF THE DRAWINGS

FIGS. 1A and 1B demonstrate that Eutrema is salt tolerant during vegetative and reproductive development. FIG. 1A shows Arabidopsis and Eutrema seeds that were germinated on soil for one week and then seedlings were left untreated (control) or treated with NaCl in 50 mM increments every three days until final concentrations (indicated) were reached. Photographs were taken after three weeks of treatment (scale bar=1 cm, right upper panel). FIG. 1B shows Arabidopsis and Eutrema that were grown in soil for three weeks and then, after the onset of inflorescence development, were left untreated (control) or treated with NaCl in 50 mM increments every three days until final concentrations (indicated) were reached. Photographs were taken after four weeks of treatment (scale bar=7 cm, upper right panel).

FIGS. 2A and 2B demonstrate that the CBL10 genes from Arabidopsis and Eutrema have different expression patterns. FIG. 2A shows RNA isolated from Arabidopsis and Eutrema roots (R) and leaves (L) of two-week-old seedlings and from stage 14 flowers (F). Elongation Factor 1a (EF1α) was used as a loading control, gels represent one image of three replicates. FIG. 2B shows the transcript structure of the CBL10 genes from Arabidopsis and Eutrema (boxes=coding sequences, lines=introns).

FIG. 3 shows transcript structure of AtCBL10, EsCBL10a, and EsCBL10b and alternatively spliced variants (black boxes=coding sequence; gray boxes=untranslated region following a premature termination codon; lines=introns; bands (upper, middle, and lower), refer to the RT-PCR bands observed in FIG. 2A; numbers, the number of times the variant was detected in 10 sequencing reactions per band). Domains known to be important for AtCBL10 function are designated (hydrophobic domain, EF-hand calcium-binding domains, and serine phosphorylation site).

FIG. 4A shows amiRNA annealing sites and sequences targeting EsCBL10a, EsCBL10b, or both EsCBL10a and EsCBL10b. (Red, underlined letters=mismatches between the amiRNA and the target gene; black boxes=coding sequence; black lines=introns). FIG. 4B shows the specificity of amiRNAs for EsCBL10a and EsCBL10b. Regions of the amiRNA sequence and number of allowable matches are designated according to Ossowoski, S., et al., The Plant Journal for Cell and Molecular Biology, 2008, 53:674-690. (Red, underlined letters=mismatches between the amiRNA and EsCBL10 gene).

FIGS. 5A-5C demonstrate that reduced expression of EsCBL10a and EsCBL10b result in seedling hypersensitivity to salt. FIG. 5A shows photographs of seedlings expressing amiRNAs designed to reduce expression of EsCBL11a and EsCBL10b singly (a1 and b1, respectively) and in combination (ab1 and ab2). Five-day-old wild-type (-, no amiRNA construct) and amiRNA seedlings were transferred to media without (control) and with 200 mM NaCl for 12 days (scale bar=1 cm, right corner). FIG. 5B shows the quantification of fresh weight, used to quantify growth. Data are means±SE of at least 30 seedlings per genotype grown in three independent experiments. FIG. 5C demonstrates the fold change in expression levels in EsCBL10a and EsCBL10b by quantitative RT-PCR. mRNA levels were normalized to EsACTIN (ΔC_{T}) and the fold change in expression relative to wild type was calculated (2^{-ΔΔCT}). Data are means±SE of three biological replicates with two technical replicates each. For all graphs, letters represent significant differences between genotypes (Tukey-Kramer HSD, P≤0.05).

FIGS. 6A and 6B demonstrate that reduced expression of EsCBL11a and EsCBL10b decreases root growth. amiRNAs designed to reduce expression of EsCBL11a and EsCBL10b singly (a1 and b1, respectively) and in combination (ab1 and ab2). Five-day-old wild-type (-, no amiRNA construct) and amiRNA seedlings were transferred to media without (control) and with 200 mM NaCl for 12 days. To analyze root growth, the number of branch roots were counted (FIG. 6A) and the length of the primary root was measured (FIG. 6B). Data are means±SE of at least 30 seedlings per genotype grown in three independent experiments. For all graphs, letters represent significant differences between genotypes (Tukey-Kramer HSD, P≤0.05).

FIGS. 7A-7C demonstrate that EsCBL10a and EsCBL10b complement the Atcbl10 salt-sensitive phenotype. FIG. 7A shows photographs of seedlings where EsCBL10a and EsCBL10b were expressed in Atcbl10 downstream of the CaMV 35S promoter. Four-day-old wild-type (WT), Atcbl10, and Atcbl10 expressing AtCBL10 (A 10), EsCBL10a (E10a), and EsCBL10b (E10b) seedlings were transferred to media without (control) and with 125 mM NaCl for 10 days (scale bar=1 cm, right corner). FIG. 7B shows the quantification of fresh weight and the length of the primary root, used to quantify growth. Data are means±SE of at least 24 seedlings per genotype grown in three independent experiments. For all graphs, letters represent significant differences between genotypes (Tukey-Kramer HSD, P≤0.05). FIG. 7C shows RNA isolated from seedlings and transcript levels compared using RT-PCR with primers designed to amplify all three CBL10 genes. ACTIN, loading control. One representative image of three replicates is shown.

FIG. 8 demonstrates that EsCBL10a and EsCBL10b complement the Atcbl10 fertilization defect. Reproductive development was examined in wild type (WT), Atcbl10, and Atcbl10 expressing EsCBL10a and EsCBL10b downstream of the CaMV 35S promoter. Plants were grown for three weeks and then left untreated (control) or treated with 40 mM NaCl for three weeks. Siliques were removed from the primary inflorescence (oldest to youngest, left to right; scale bar=1.5 cm, upper right corner). One representative image of two replicates is shown.

FIGS. 9A-9C demonstrate that EsCBL10b strongly activates the Arabidopsis and Eutrema SOS pathways. A salt-sensitive strain of Saccharomyces cerevisiae (AXT3K, Δena1-4Δnha1Δnhx1) was transformed with SOS1 and SOS2 from Arabidopsis (FIG. 9A) or Eutrema (FIG. 9B) in combination with AtCBL10 (A 10), EsCBL10a (E10a), and EsCBL10b (E10b). Serial decimal dilutions of yeast cells were spotted onto control media or media containing 125 mM NaCl (FIG. 9A) or 250 mM NaCl (FIG. 9B) and grown for four days at 30° C. Two independently transformed colonies were assayed in three biological replicates. FIG. 9C shows RNA isolated and transcript levels compared using RT-PCR with primers that amplify all three CBL10 genes. Yeast 18S rRNA, loading control. One representative image of three replicates is shown.

FIG. 9D demonstrates that EsCBL10b and AtCBL10 interact more strongly with AtSOS2 than EsCBL10a. AtCBL10 (A10), EsCBL10a (E10a), EsCBL10b (E10b), and AtSOS3 (A3) were fused to the GAL4 DNA-activation domain (AD) and interaction with AtSOS2 fused to the GAL4 DNA-binding domain (BD) was assessed using yeast two-hybrid assays. Serial decimal dilutions of diploid yeast harboring both constructs were spotted onto synthetic defined media (SD) minus leucine (L) and tryptophan (W), minus LW and histidine (H), minus LWH and adenine (A), or with the addition of 0.5 mM 3-amino-1,2,4-triazole 3-AT (3AT). Two independently mated colonies were assayed in two biological replicates; one representative image is shown.

FIG. 9E demonstrates that EsCBL10b interaction with AtSOS2 is orientation specific. AtCBL10 (A10), EsCBL10a (E10a), EsCBL10b (E10b), and AtSOS3 (A3) were fused to the GAL4 DNA-binding domain (BD) and interaction with AtSOS2 fused to the GAL4 DNA-activation domain (AD) was assessed using yeast two-hybrid assays. Serial decimal dilutions of diploid yeast harboring both constructs were spotted onto synthetic defined media (SD) minus leucine (L) and tryptophan (W), minus LW and histidine (H), minus LWH and adenine (A), or with the addition of 0.5 mM 3-amino-1,2,4-triazole 3-AT (3AT). Two independently mated colonies were assayed in two biological replicates; one representative image is shown.

FIGS. 10A-10C demonstrate that EsCBL10a but not EsCBL10b complements the Atsos3 salt-sensitive phenotype. FIG. 10A shows photographs of seedlings where EsCBL10a, EsCBL10b and EsSOS3 were expressed in Atsos3 downstream of the CaMV 35S promoter. Four-day-old wild-type (WT), Atsos3, and Atsos3 expressing AtCBL10 (A10), EsCBL10a (E10a), EsCBL10b (E10b), AtSOS3 (A3), and EsSOS3 (E3) seedlings were transferred to media without (control) or with 75 mM NaCl for 10 days (scale bar=1 cm, upper right). FIG. 10B shows the quantification of fresh weight and the length of the primary root, used to quantify growth. Data are means±SE of at least 24 seedlings per genotype grown in three independent experiments. For all graphs, letters represent significant differences between genotypes (Tukey-Kramer HSD, P≤0.05). FIG. 10C shows RNA isolated from seedlings and transcript levels compared using RT-PCR with primers designed to amplify all three CBL10 or both SOS3 genes. ACTIN, loading control. One representative image of three replicates is shown.

FIG. 10D. Four Arabidopsis CIPKs interact with EsCBL10a. AtCBL10 (A10), EsCBL10a (E10a), EsCBL10b (E10b), and AtSOS3 (A3) were fused to the GAL4 DNA-activation domain (AD) and interaction with the Arabidopsis CIPK proteins fused to the GAL4 DNA-binding domain (BD) was assessed using yeast two-hybrid assays. Serial decimal dilutions of diploid yeast harboring both constructs were spotted onto synthetic defined media (SD) minus leucine (L) and tryptophan (W) or minus LW and histidine (H). Two independently mated colonies were assayed in two biological replicates; one representative image is shown.

FIG. 10E. Orientation of the yeast two-hybrid affects CBL10 interaction with the CIPK proteins. AtCBL10 (A10), EsCBL10a (E10a), EsCBL10b (E10b), and AtSOS3 (A3) were fused to the GAL4 DNA-binding domain (BD) and interaction with the Arabidopsis CIPK proteins fused to the GAL4 DNA-activation domain (AD) was assessed using yeast two-hybrid assays. Serial decimal dilutions of diploid yeast harboring both constructs were spotted onto synthetic defined media (SD) minus leucine (L) and tryptophan (W) or minus LW and histidine (H). Two independently mated colonies were assayed in two biological replicates; one representative image is shown.

FIGS. 11A-11D demonstrate EsSOS3 activates the Arabidopsis and Eutrema SOS pathways. A salt-sensitive strain of Saccharomyces cerevisiae (AXT3K, Δena1-4Δnha1Δnhx1) was transformed with SOS1 and SOS2 from Arabidopsis (FIGS. 11A and 11C) or Eutrema (FIGS. 11B and 11D) in combination with AtCBL10 (A10), EsCBL10a (E10a), EsCBL10b (E10b), AtSOS3 (A3), and EsSOS3 (E3). FIGS. 11A and 11B show serial decimal dilutions of yeast cells spotted onto media without (control) or with 125 mM NaCl (FIG. 11A) or 250 mM NaCl (FIG. 11B) and grown for four days at 30° C. Two independently transformed colonies were assayed in three biological replicates. FIGS. 11C and 11D show RNA isolated and transcript levels compared using RT-PCR with primers that amplify all three CBL10 or both SOS3 genes. 18S rRNA, loading control. One representative image of three replicates is shown.

FIGS. 12A-12C demonstrate that EsSOS3 does not complement the Atcbl10 salt-sensitive phenotype. FIG. 12A shows photographs of seedlings where EsSOS3 was expressed in Atcbl10 downstream of the CaMV 35S promoter. Four-day-old wild-type (WT), Atcbl10, and Atcbl10 expressing AtCBL10 (A10), AtSOS3 (A3), and EsSOS3 (E3) seedlings were transferred to media without (control) and with 125 mM NaCl for 10 days (scale bar=1 cm, right panel). FIG. 12B shows the quantification of fresh weight and the length of the primary root, used to quantify growth. Data are means±SE of at least 24 seedlings per genotype grown in three independent experiments. For all graphs, letters represent significant differences between genotypes (Tukey-Kramer HSD, P≤0.05). FIG. 12C shows RNA isolated from seedlings and transcript levels compared using RT-PCR with primers that amplify both SOS3 genes. ACTIN, loading control. One representative image of three replicates is shown.

FIGS. 13A-13C demonstrate that EsCBL10a and EsCBL10b can confer salt tolerance. FIG. 13A shows photographs of seedlings where Atcbl10 plants strongly expressing EsCBL10a (E10a) and EsCBL10b (E10b) singly downstream of the CaMV 35S promoter were crossed to generate heterozygous plants expressing both genes (double, abD). Four-day-old seedlings were transferred to media without (control) and with 125 mM NaCl for 10 days (scale bar=1 cm, right panel). FIG. 13B shows the quantification of fresh weight and the length of the primary root, used to quantify growth. Data are means±SE of at least 48 seedlings from six independent crosses grown in three independent experiments. For all graphs, letters represent significant differences between genotypes (Tukey-Kramer HSD, P≤0.05). FIG. 13C shows RNA isolated from seedlings and transcript levels compared using RT-PCR with primers that specifically amplify each gene. ACTIN, loading control. One representative image of three replicates is shown.

FIGS. 14A-14C demonstrate that EsCBL10a and EsSOS3 significantly enhance root growth. FIG. 14A shows photographs of plants expressing EsCBL10a (E10a) and EsSOS3 (E3) singly downstream of the CaMV 35S promoter. These plants were crossed to generate heterozygous plants expressing both genes (double, a3D). Four-day-old seedlings were transferred to media without (control) and with 75 mM NaCl for 10 days (scale bar=1 cm, upper right panel). FIG. 14B shows the quantification of fresh weight and the length of the primary root, used to quantify growth. Data are means±SE of at least 48 seedlings from six independent crosses grown in three independent experiments. For all graphs, letters represent significant differences between genotypes (Tukey-Kramer HSD, P≤0.05). FIG. 14C shows RNA isolated from seedlings and transcript levels compared using RT-PCR with primers that specifically amplify each gene. ACTIN, loading control. One representative image of three replicates is shown.

FIGS. 15A-15C demonstrate that EsCBL10a and EsSOS3 significantly enhance root growth. FIG. 15A shows photographs of a second set of plants expressing EsCBL10a (E10a) and EsSOS3 (E3) singly downstream of the CaMV 35S promoter were crossed to generate heterozygous plants expressing both genes (double, a3D). Four-day-old seedlings were transferred to media without (control) and with 75 mM NaCl for 10 days. FIG. 15B shows the quantification of fresh weight and the length of the primary root, used to quantify growth. Data are means±SE of at least 48 seedlings from six independent crosses grown in three independent experiments. For all graphs, letters represent significant differences between genotypes (Tukey-Kramer HSD, P≤0.05). FIG. 15C shows RNA isolated from seedlings and transcript levels compared using RT-PCR with primers that specifically amplify each gene. ACTIN, loading control. One representative image of three replicates is shown.

FIG. 16 is a schematic showing that the duplication of EsCBL10 increased the complexity of signaling in response to salt. Without wishing to be bound by theory, as shown in the schematic, in Arabidopsis, SOS3 and CBL10 interact with and activate the protein kinase SOS2 which, in turn, activates the sodium-proton exchanger SOS1 (SOS pathway) at the plasma membrane (PM). SOS1 then transports sodium out of the cell preventing its toxic accumulation. SOS3 and CBL10 also appear to have additional functions outside of activation of the SOS pathway. Homologs were identified in Eutrema and one gene, CBL10, has been duplicated. EsCBL10b has an enhanced ability to activate SOS2 and SOS1 from Arabidopsis and Eutrema and complements the Atcbl10 salt-sensitive phenotype. EsCBL10a complements both the Atcbl10 and Atsos3 salt-sensitive phenotypes but only weakly activates the SOS pathway. EsSOS3 activates SOS2 and SOS1 from Arabidopsis and Eutrema and complements the Atsos3 salt-sensitive phenotype.

FIGS. 17A-17C show nucleotide and amino acid comparisons. FIG. 17A shows a comparison of CBL10 and SOS3 nucleotides (% identity). FIG. 17B shows a comparison of CBL10 and SOS3 proteins. FIG. 17C shows an alignment of CBL10 and SOS3 proteins. Nucleotide sequences from Arabidopsis and Eutrema were aligned by codon and translated to amino acids. Domains known to be important for AtCBL10 function are designated. Hydrophobic domain, amino acids with hydrophobic side chains that likely target AtCBL10 to a membrane; Ca′, EF-hand calcium-binding domains; S, serine phosphorylation site; red letters, amino acid differences between CBL10 proteins or between SOS3 proteins; blue boxes, amino acid changes that might underlie the difference in EsCBL10 function.

FIGS. 18A-18C demonstrate that the first eight amino acids of EsCBL10b are important for activation of the SOS pathway. FIG. 18A: Yeast (AXT3K) was transformed with Arabidopsis SOS1 and SOS2 in combination with AtCBL10 (A10), EsCBL10a (E10a), EsCBL10b (E10b) or the chimeric genes. Red, E10a protein; blue, E10b protein; I, insertion of seven amino acids in E10a; HD, hydrophobic domain; V, variable sequence of amino acids; Ca, calcium-binding domains; S, serine phosphorylation site. Serial decimal dilutions of yeast cells were spotted onto control media or media containing 125 mM NaCl. Two independently transformed colonies were assayed in three biological replicates; one representative image is shown. FIG. 18B: Transcript accumulation. Yeast/8S rRNA, loading control. One representative image of three replicates is shown. FIG. 18C: Amino acids in the N3 fragment were aligned and color coded based on side chain properties. Blue, non-polar (for E10b, blue amino acids (i.e., non-polar amino acids) are the underlined amino acids in MDW-------PRFSS; for A10 blue amino acids are the underlined amino acids in ME---------QVSS; for E10a blue amino acids are the underlined amino acids in MVPVNQCLLDPKVSS); magenta, polar (for E10b, magenta amino acids (i.e., polar amino acids) are the underlined amino acids in MDW-------PRFSS; for A10 magenta amino acids are the underlined amino acids in ME-------QVSS; for E10a magenta amino acids are the underlined amino acids in MVPVNQCLLDPKVSS); green, negatively charged (for E10b, green amino acids (i.e., negatively charged amino acids) are the underlined amino acids in MDW-------PRFSS; for A10 green amino acids are the underlined amino acids in ME QVSS; for E10a green amino acids are the underlined amino acids in MVPVNQCLLDPKVSS); orange, positively charged (for E10b, orange amino acids (i.e., positively charged amino acids) are the underlined amino acids in MDW PRFSS; for A10, there are no orange amino acids; for E10a orange amino acids are the underlined amino acids in MVPVNQCLLDPKVSS).

FIGS. 19A-19B: Chimeric EsCBL10 proteins. EsCBL10a and EsCBL10b were divided into an N-terminus (N1, N2, and N3) and a C-terminus (C1, C2, and C3) and the termini exchanged to generate chimeric proteins. Domains important for CBL10 function are indicated: I, insertion of seven amino acids in EsCBL10a; HD, hydrophobic domain; V, variable sequence of amino acids; Ca, calcium-binding domains; S, serine phosphorylation site. FIG. 19A: Schematic representation of the chimeric proteins. Red, EsCBL10a or 10a; blue, EsCBL10b or 10b. Chimeric proteins are designated based on the N-terminus. FIG. 19B: Amino acid alignment of the CBL10 proteins with chimeric termini indicated with black lines. Domains important for AtCBL10 function are indicated (I, HD, V, Ca, S). Underlined letters indicate amino acid differences.

FIGS. 20A-20C: The chimeric EsCBL10 genes are expressed in the Atcbl10 and Atsos3 mutants. Gene specific primers were used to confirm the identity of the chimeric genes (FIG. 20A) and primers amplifying all genes were used to examine transcript accumulation (FIGS. 20B-20C). ACTIN, loading control. One representative image of three replicates is shown.

FIGS. 21A-21C: All chimeric proteins complement the Atcbl10 salt-sensitive phenotype. Chimeric EsCBL10a (E10a)/EsCBL10b (E10b) proteins were expressed in Atcbl10 and growth in the absence (control) and presence of salt (125 mM NaCl) was monitored. FIG. 21A: Schematic representation of the chimeric proteins. Red, E10a protein; blue, E10b protein; I, insertion of seven amino acids in E10a; HD, hydrophobic domain; V, variable sequence of amino acids; Ca, calcium-binding domains; S, serine phosphorylation site. FIG. 21B: Photographs of wild type (WT), Atcbl10, and Atcbl10 expressing E10a, E10b, or the chimeric proteins. Bar (1 cm, upper right panel) shows magnification for all images. FIG. 21C: Fresh weight was measured to quantify growth. Data are means±standard error of at least 24 seedlings per genotype grown in three independent experiments. For all graphs, different letters indicate significant differences between genotypes (two-way ANOVA, Tukey-Kramer HSD, P≤0.05).

FIGS. 22A-22D: The hydrophobic domain of EsCBL10a is important for complementation of Atsos3. Chimeric EsCBL10a (E10a)/EsCBL10b (E10b) proteins were expressed in Atsos3 and growth in the absence (control) and presence of salt (75 mM NaCl) was monitored. FIG. 22A: Schematic representation of the chimeric proteins. Red, E10a protein; blue, E10b protein; I, insertion of seven amino acids in E10a; HD, hydrophobic domain; V, variable sequence of amino acids; Ca, calcium-binding domains; S, serine phosphorylation site. FIG. 22B: Photographs of wild type (WT), Atsos3, and Atsos3 expressing E10a, E10b, or the chimeric proteins. Bar (1 cm, upper right) shows magnification for all images. FIG. 22C: Fresh weight and length of the primary root were measured to quantify growth. Data are means±standard error of at least 24 seedlings per genotype grown in three independent experiments. For all graphs, different letters indicate significant differences between genotypes (two-way ANOVA, Tukey-Kramer HSD, P≤0.05). FIG. 22D: Amino acids in the N-terminus of E10a, E10b, AtCBL10 (A10), and AtSOS3 (A3) proteins were aligned and color coded based on side chain properties. Blue, non-polar (for E10a, blue amino acids (i.e., non-polar amino acids) are the underlined amino acids in

for A3, blue amino acids (i.e., non-polar amino
acids) are the underlined amino acids in MGCSV-
SKKKKKNAMRPP-
MVPVNQCLLDPKVSSRSSSLTVGEQICAVFIPFFAVVDFLFSTMGQCFDC
HRRRRRRL-PQTCQHADLARLARESRFSINEVE;
for E10b, blue amino acids (i.e., non-polar amino
acids) are the underlined amino acids in
MDW-------
GYEDPELLASVTPFTVEEVE;
for A10, blue amino acids (i.e., non-polar amino
acids) are the underlined amino acids in
ME---------
PRFSSRSSSLTVGEKICAVFIPLIAIIDILFSTVGQCFDCCRVP-NRS-
SQTCQHLDLAHLARESRFSINEVE;
magenta, polar (for E10a, magenta amino acids
(i.e., polar amino acids) are the underlined amino
acids in MVPVNQCLLDPKVSSRSSSLTVGEQICAVFIPFFAVVDFLF
STMGQCFDCHRRRRRRL
QVSSRSSSLTVGEQFCAVFIPFFAIIDVLVSSVGQCFDCR----STS-
PRTCQHADLERLARESQFSVNEVE);
for A3, magenta amino acids (i.e., polar amino
acids) are the underlined amino acids in MGCSV-
SKKKKKNAMRPP-
-PQTCQHADLARLARESRFSINEVE;
for E10b, magenta amino acids (i.e., polar amino
acids) are the underlined amino acids in
MDW-------
GYEDPELLASVTPFTVEEVE;
for A10, magenta amino acids (i.e., polar amino
acids) are the underlined amino acids in
ME---------
PRFSSRSSSLTVGEKICAVFIPLIAIIDILFSTVGQCFDCCRVP-NRS-
SQTCQHLDLAHLARESRFSINEVE;
green, negatively charged (for E10a, green amino
acids (i.e., negatively charged amino acids) are
the underlined amino acids in MVPVNQCLLDPKVSSRSS
SLTVGEQICAVFIPFFAVVDFLFSTMGQCFDCHRRRRRRL
QVSSRSSSLTVGEQFCAVFIPFFAIIDVLVSSVGQCFDCR----STS-
PRTCQHADLERLARESQFSVNEVE);
for A3, green amino acids (i.e., negatively
charged amino acids) are the underlined amino
acids in MGCSV-SKKKKKNAMRPP-
-PQTCQHADLARLARESRFSINEVE;
for E10b, green amino acids (i.e., negatively
charged amino acids) are the underlined amino
acids in MDW-------
GYEDPELLASVTPFTVEEVE;
for A10, green amino acids (i.e., negatively
charged amino acids) are the underlined amino
acids in ME---------
PRFSSRSSSLTVGEKICAVFIPLIAIIDILFSTVGQCFDCCRVP-NRS-
SQTCQHLDLAHLARESRFSINEVE;
orange, positively charged (for E10a, orange amino
acids (i.e., positively charged amino acids) are
the underlined amino acids in MVPVNQCLLDPKVSSRSS
SLTVGEQICAVFIPFFAVVDFLFSTMGQCFDCHRRRRRRL
QVSSRSSSLTVGEQFCAVFIPFFAIIDVLVSSVGQCFDCR----STS-
PRTCQHADLERLARESQFSVNEVE);
for A3, orange amino acids (i.e., positively
charged amino acids) are the underlined amino
acids in MGCSV-SKKKKKNAMRPP-
-PQTCQHADLARLARESRFSINEVE;
for E10b, orange amino acids (i.e., positively
charged amino acids) are the underlined amino
acids in MDW-------
GYEDPELLASVTPFTVEEVE;
for A10, orange amino acids (i.e., positively
charged amino acids) are the underlined amino
acids in ME---------
PRFSSRSSSLTVGEKICAVFIPLIAIIDILFSTVGQCFDCCRVP-NRS-
SQTCQHLDLAHLARESRFSINEVE;
QVSSRSSSLTVGEQFCAVFIPFFAIIDVLVSSVGQCFDCR----STS-
PRTCQHADLERLARESQFSVNEVE).

Major element, portion of the N2 fragment conferring full ability to complement Atsos3; minor element, portion of the N1 fragment conferring partial ability to complement Atsos3; black boxes, amino acid differences that might underlie complementation.

FIGS. 23A-23C: EsSOS3 activates the Arabidopsis and Eutrema SOS pathways. FIGS. 23A and 23B: Yeast (AXT3K) was transformed with SOS1 and SOS2 from Arabidopsis (A) or Eutrema (B) in combination with AtCBL10 (A10), EsCBL10a (E10a), and EsCBL10b (E10b), AtSOS3 (A3), and EsSOS3 (E3). Serial decimal dilutions of yeast cells were spotted onto control media or media containing 125 mM NaCl (FIG. 23A) or 250 mM NaCl (FIG. 23B). Two independently transformed colonies were assayed in three biological replicates; one representative image is shown. FIG. 23C: Transcript accumulation. Yeast 18S rRNA, loading control. One representative image of three replicates is shown.

### DETAILED DESCRIPTION

### I. Introduction

The present technology relates to the identification and characterization of the gene, EsCBL10, that was found to be duplicated (EsCBL10a and EsCBL10b) and linked to salt tolerance in Eutrema salsugineum. The nucleic acid sequences of the EsCBL10a and EsCBL10b genes have been determined. The full-length sequence of EsCBL10a, including the coding region and its 5′ and 3′ upstream and downstream regulatory sequences, is set forth in SEQ ID NO: 1. The open reading frame (ORF) of SEQ ID NO: 1 is set forth in SEQ ID NO: 2. The ORF of SEQ ID NO: 2 encodes the EsCBL10a polypeptide sequence set forth in SEQ ID NO: 3. The full-length sequence of EsCBL10b, including the coding region and its 5′ and 3′ upstream and downstream regulatory sequences, is set forth in SEQ ID NO: 4. The open reading frame (ORF) of SEQ ID NO: 4 is set forth in SEQ ID NO: 5. The ORF of SEQ ID NO: 5 encodes the EsCBL10b polypeptide sequence set forth in SEQ ID NO: 6. As demonstrated herein, EsCBL10a and EsCBL10b gene products are useful in methods for increasing the salt tolerance of plants in need thereof.

The present technology also relates to EsCBL10 chimeric proteins that are useful in methods for increasing salt tolerance in plants in need thereof. Portions of EsCL10a and EsCBL10b were exchanged to generate three sets of chimeric proteins. The first chimeric proteins fused the amino-terminal half of one of the EsCBL10 proteins with the carboxy-terminal half of the other (aN1 (SEQ ID NOs: 10, 11) and bN1 (SEQ ID NOs: 16, 17)). The second (aN2 (SEQ ID NOs: 12, 13) and bN2 (SEQ ID NOs: 18, 19)) and third (aN3 (SEQ ID NOs: 14, 15) and bN3 (SEQ ID NOs: 20, 21)) sets of chimeric proteins progressively narrowed down the region underlying the specific functions. In addition, the present technology relates to the identification and characterization of regions of EsCBL10 important for conferring salt tolerance. For example, the EsCBL10a major element (SEQ ID NOs: 22, 23) and minor element (SEQ ID NOs: 24, 25).

Salt, particularly sodium chloride (NaCl), negatively affects plant growth in several ways. The accumulation of salt in the soil restricts water movement into the plant (osmotic stress) and the accumulation of sodium ions in a plant cell interferes with metabolic and photosynthetic processes (ionic stress) (Munns & Tester, 2008). Molecular studies in Arabidopsis thaliana (Arabidopsis) have identified genes important for alleviating ionic stress and provided insight into the pathways in which these genes function. The Arabidopsis SALT-OVERLY-SENSITIVE (SOS) pathway removes sodium from the cell preventing its toxic accumulation in the cytosol. In this pathway, accumulation of sodium triggers an influx in cytosolic calcium. Increased calcium is perceived by two calcium sensors, SOS3 (roots) and CALCINEURIN B-LIKE10 (CBL10, also known as SOS3-LIKE CALCIUM-BINDING PROTEIN8, leaves), which bind to and activate the SOS2 serine/threonine protein kinase (Liu & Zhu, 1998; Halfter, et al., 2000; Liu, et al. 2000; Kim, et al., 2007; Quan, 2007). SOS2 phosphorylates SOS1 a sodium/proton exchanger, initiating transport of sodium out of the cell (Shi, et al., 2000; Qiu, et al., 2002; Quintero, et al., 2002; Qiu, et al., 2003).

Despite advances in identifying Arabidopsis genes important during growth in the presence of salt, growth of Arabidopsis, along with many crop plants, is significantly reduced when salt is present in the soil (glycophytes) (Munns & Tester, 2008). Halophytes are plants that can maintain growth for longer in higher concentrations of salt, but little is known about the mechanisms underlying their adaptation (Munns & tester, 2008). Eutrema salsugineum (Eutrema, formerly Thellungiella halophila) is a halophytic relative of Arabidopsis (Bressan, et al., 2001; Inan, et al., 2004; Amtmann, 2009). Eutrema is a small crucifer native to the seashore saline soils of Eastern China. Eutrema does not require salt for optimal growth but is able to survive in conditions that kill most, if not all, crop plants and its tolerance does not appear to rely on specialized morphological structures (Inan, et al., 2004; Amtmann, 2009).

Comparative genomic studies reveal that plant gene families are largely conserved across species, indicating that plants amplify and modify a basic set of genes rather than developing new gene families (Flagel & Wendel, 2009). This tendency to use a basic suite of genes suggested that the SOS pathway, identified in Arabidopsis, might also function in Eutrema with modifications that contribute to Eutrema's increased salt tolerance. Several studies have compared the activity of the SOS1 sodium/proton exchanger in Arabidopsis (AtSOS1) and Eutrema (EsSOS1). When the two proteins were expressed in a salt-sensitive strain of Saccharomyces cerevisiae (yeast), yeast expressing EsSOS1 grew better in the presence of salt than yeast expressing AtSOS1, indicating that EsSOS1 has a greater level of self-activation than AtSOS1 (Oh, et al., 2009; Jarvis, et al., 2014). When the entire SOS pathways were reconstructed in this salt-sensitive yeast strain, yeast expressing the Eutrema SOS pathway (EsSOS3, EsSOS2, and EsSOS1) was more salt tolerant than yeast expressing the Arabidopsis SOS pathway (AtSOS3, AtSOS2, and AtSOS1) (Jarvis, et al., 2014). From this study, it was not possible to determine whether the greater growth of yeast expressing the Eutrema SOS pathway is due to EsSOS1 alone or whether EsSOS2 and EsSOS3 can more effectively activate EsSOS1.

The disclosure of the present technology relates to the identification and characterization of additional Eutrema SOS pathway proteins and one, CBL10 (EsCBL10), was found to be duplicated (EsCBL10a and EsCBL10b). Gene duplication is a major source of genetic diversity and, consequently, adaptive evolution (Ohno, 1970; Kondrashov, 2012). Because most genes are lost via pseudogenization, the maintenance of duplicated gene pairs in a genome can be indicative of adaptive benefit conferred by the paralogous genes (Hah, 2009; Kondrashov, 2012). The disclosure of the present technology demonstrates that the duplication of CBL10 contributes to Eutrema's salt tolerance.

Because Eutrema maintains growth in salt-affected soils that kill most crop plants, the duplication of CBL10 provides a unique opportunity to functionally test the outcome of gene duplication and its link to plant salt tolerance. To understand the roles the duplicated Eutrema CBL10 genes play in response to salinity, their expression patterns were monitored and their expression was reduced individually and in combination. To determine the extent of divergence in CBL10 function after duplication and to uncover elements of the signaling pathways in which the proteins function, protein activities were examined using cross-species complementation and yeast two-hybrid assays. To identify domains and amino acids responsible for the divergence in function, chimeric EsCBL10 proteins were generated.

As described herein, it was found that, in Eutrema, down-regulation of either of the duplicated EsCBL10 genes decreased growth in the presence of salt suggesting that both EsCBL10a and EsCBL10b function in Eutrema's response to salinity. Down-regulation of both EsCBL10 genes in combination led to an even greater decrease in growth, suggesting the genes have additive effects or different functions. Cross-species complementation assays revealed that EsCBL10b has an enhanced ability to activate the SOS pathway while EsCBL10a has a function not performed by Arabidopsis CBL10 (AtCBL10) or EsCBL10b. Four kinases that interact with EsCBL10a were identified and chimeric proteins revealed that the role of EsCBL10b in the SOS pathway and the role of EsCBL10a in an alternative pathway were the result of changes in the N-termini of the proteins. Analysis of the EsSOS3 calcium sensor revealed that EsCBL10a and EsSOS3 have partially overlapping functions, but also distinct roles in salt tolerance. The duplication of EsCBL10 appears to have increased the calcium-mediated signaling capacity in Eutrema and the duplicated genes conferred increased salt tolerance when expressed in salt-sensitive Arabidopsis. Accordingly, as described herein, EsCBL10a and EsCBL10b confer salt tolerance in E. salsugineum and can improve the growth of glycophytes, such as Arabidopsis thaliana, in hypersaline conditions.

Thus, in some embodiments, the present technology provides an EsCBL10a and EsCBL10b gene or biologically active fragments thereof, or EsCBL10 chimeric protein nucleic acids that may be used to genetically engineer plants to have increased salt tolerance. In some embodiments, a salt-sensitive crop plant may be engineered to express EsCBL10a and/or EsCBL10b proteins or biologically active fragments thereof, or EsCBL10 chimeric proteins to increase salt tolerance (e.g., increased growth, as measured by, e.g., fresh weight, root length under hypersaline conditions) in the plant. In some embodiments, a glycophytic plant such as Arabidopsis thaliana can be genetically engineered to express EsCBL10a and/or EsCBL10b proteins or biologically active fragments thereof, or EsCBL10 chimeric proteins to increase salt tolerance (e.g., increased growth, as measured by, e.g., fresh weight, root length under hypersaline conditions) in the plant.

### II. Definitions

All technical terms employed in this specification are commonly used in biochemistry, molecular biology, and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “biologically active fragment” means a fragment of EsCBL10a, EsCBL10b, or EsSOS3 which can, for example, bind to an antibody that will also bind the full length EsCBL10a, EsCBL10b, or EsSOS3, respectively. In some embodiments, a biologically active fragment of EsCBL10a, EsCBL10b, or EsSOS3 can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the full length sequence (either amino acid or nucleic acid).

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active protein. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

As used herein, “EsCBL10 chimeric proteins” include aN1 (SEQ ID NOs: 10, 11), bN1 (SEQ ID NOs: 16, 17), aN2 (SEQ ID NOs: 12, 13), bN2 (SEQ ID NOs: 18, 19), aN3 (SEQ ID NOs: 14, 15), and bN3 (SEQ ID NOs: 20, 21).

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant (e.g., “EsCBL10 overexpression”).

As used herein, “expression cassette” refers to a nucleic acid construct comprising a nucleotide sequence of interest (e.g., the nucleotide sequences of the present technology), wherein the nucleotide sequence is operatively associated with at least a control sequence (e.g., a promoter). Thus, some embodiments of the present technology provide expression cassettes designed to express the nucleotide sequences of the present technology. In this manner, for example, one or more plant promoters operatively associated with one or more nucleotide sequences of the present technology (e.g., SEQ ID NOs: 1, 2, 4, 5, 7, 8, 10, 12, 14, 16, 18, 20, 22, and/or 24) are provided in expression cassettes for expression in an organism or cell thereof (e.g., a plant, plant part, and/or plant cell).

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event.

“Genetic engineering” encompasses any methodology for introducing a nucleic acid into a host organism. For example, a plant is genetically engineered when a polynucleotide sequence is introduced into the plant that results in the expression of a novel gene in the plant, or an increase in the level of a gene product that is naturally found in the plants. In the present context, “genetically engineered” includes transgenic plants and plant cells, as well as plants and plant cells produced by the methods disclosed herein. “Transgenic plant” includes reference to a plant that comprises within its genome a heterologous polynucleotide. In some embodiments, the heterologous polynucleotide is stably integrated within the genome such that the polynucleotide is passed on to successive generations. In some embodiments, the heterologous polynucleotide may be integrated into the genome alone or as part of a recombinant expression cassette. “Transgenic” may include any cell, cell line, callus, tissue, plant part, or plant, the genotype of which has been altered by the presence of heterologous nucleic acid including those transgenics initially so altered as well as those created by sexual crosses or asexual propagation from the initial transgenic.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

“Increased salt tolerance” or “increasing salt tolerance” refers to a plant that exhibits the ability to grow in a manner that is more successful than comparable control plants that are not genetically engineered as described herein, even under circumstances or conditions of increased salinity (e.g., increases in soil or water salinity (e.g., NaCl)) for the plant. In other words, the genetically engineered plant is able to develop more or less normally with respect to growth rate (e.g., root length), biomass (e.g., fresh weight), color, maturation, fruit production, etc., even under conditions of increased salinity. Under adverse, increased salinity conditions, plants that are genetically engineered according to the methods of the present technology maintain a growth rate and/or accumulate biomass and/or achieve a final biomass that is at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater than control plants grown under similar conditions.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores. The class of plants that can be used in the methods of the present technology is generally as broad as the class of higher plants amenable to transformation techniques, including monocotyledonous and dicotyledonous plants, as well as gymnosperms.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” Examples of promoters include, but are not limited to, a CaMV 35S promoter, a CaMV 19S promoter, a viral coat protein promoter, a monocot promoter, a ubiquitin promoter, an actin promoter, a cab promoter, a sucrose synthase promoter, a tubulin promoter, a napin R gene complex promoter, a tomato E8 promoter, a patatin promoter, a mannopine synthase promoter, a soybean seed protein glycinin promoter, a soybean vegetative storage protein promoter, a bacteriophage SP6 promoter, a bacteriophage T3 promoter, a bacteriophage T7 promoter, a Ptac promoter, a root-cell promoter, an ABA-inducible promoter, and a turgor-inducible promoter. “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software.

A “vector” or “expression vector” refers to a composition for transferring, delivering, or introducing a nucleic acid (or nucleic acids) into a cell. A vector comprises a nucleic acid molecule comprising the nucleotide sequence(s) to be transferred, delivered, or introduced. Vectors for use in the transformation of plants are well known in the art.

### III. Increasing Salt Tolerance in Plants

The disclosure of the present technology relates to the use of EsCBL10a, EsCBL10b, and/or EsSOS3 or biologically active fragments thereof in compositions and methods for increasing salt tolerance in plants.

A. Genetic Engineering of Plants and Cells Using EsCBL10a, EsCBL10b, EsCBL10 Chimeric Protein, and/or EsSOS3 Sequences that Confer Salt Tolerance.

Sequences

Genes of the present technology conferring salt tolerance in plants include the sequences set forth in SEQ ID NOs: 2, 4, 5, 7, 8, 10, 12, 14, 16, 18, 20, 22, and 24, and nucleic acids encoding the polypeptides set forth in SEQ ID NOs: 3, 6, 9, 11, 13, 15, 17, 19, 21, 23, and 25 including biologically active fragments thereof.

Biologically active fragments of SEQ ID NO: 1 may include fragments of at least about 15 contiguous nucleic acids up to about 1930 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375; about 1400; about 1425; about 1450; about 1475; about 1500; about 1525; about 1550; about 1575; about 1600; about 1625; about 1650; about 1675; about 1700; about 1725; about 1750; about 1775; about 1800; about 1825; about 1850; about 1875; about 1900; about 1910, or about 1920 contiguous nucleic acids.

Biologically active fragments of SEQ ID NO: 2 may include fragments of at least about 15 contiguous nucleic acids up to about 775 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 760, or about 770 contiguous nucleic acids.

Biologically active fragments of SEQ ID NO: 4 may include fragments of at least about 15 contiguous nucleic acids up to about 2000 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375, about 1400, about 1425, about 1450, about 1475, about 1500, about 1525, about 1550, about 1575, about 1600, about 1625, about 1650, about 1675, about 1700, about 1725, about 1750, about 1775, about 1800, about 1825, about 1850, about 1875, about 1900, about 1925, about 1950, about 1960, about 1970, about 1980, or about 1990 contiguous nucleic acids.

Biologically active fragments of SEQ ID NO: 5 may include fragments of at least about 15 contiguous nucleic acids up to about 750 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 735, or about 745 contiguous nucleic acids.

Biologically active fragments of SEQ ID NO: 7 may include fragments of at least about 15 contiguous nucleic acids up to about 1500 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375, about 1400, about 1425, about 1450, about 1460, about 1470, about 1480, or about 1490 contiguous nucleic acids.

Biologically active fragments of SEQ ID NO: 8 may include fragments of at least about 15 contiguous nucleic acids up to about 660 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 635, about 645, or about 655 contiguous nucleic acids.

The present technology also includes “variants” of SEQ ID NOs: 1, 2, 4, 5, 7, 8, 10, 12, 14, 16, 18, 20, 22, and 24, with one or more bases deleted, substituted, inserted, or added, which variant codes for a polypeptide that confers salt tolerance in a plant. Accordingly, sequences having base sequences with one or more bases deleted, substituted, inserted, or added retain physiological activity even when the encoded amino acid sequence has one or more amino acids substituted, deleted, inserted, or added. Additionally, multiple forms of EsCBL10a, EsCBL10b, EsSOS3, EsCBL10 chimeric protein, or EsCBL10a major or minor elements may exist, which may be due to post-translational modification of a gene product, or to multiple forms of the transcription factor gene. Nucleotide sequences that have such modifications and that code for an EsCBL10a, EsCBL10b, or EsSOS3 protein, or EsCBL10 chimeric protein, or EsCBL10a major or minor element that confers salt tolerance in a plant are included within the scope of the present technology.

For example, the poly A tail or 5′- or 3′-end, nontranslated regions may be deleted, and bases may be deleted to the extent that amino acids are deleted. Bases may also be substituted, as long as no frame shift results. Bases also may be “added” to the extent that amino acids are added. However, it is essential that any such modification does not result in the loss of activity that confers salt tolerance.

An EsCBL10a, EsCBL10b, EsCBL10 chimeric protein, EsCBL10a major or minor element, or EsSOS3 sequence can be synthesized ab initio from the appropriate bases, for example, by using an appropriate protein sequences disclosed herein as a guide to create a DNA molecule that, though different from the native DNA sequence, results in the production of a protein with the same or similar amino acid sequence.

The present technology encompasses nucleic acid molecules which are at least about 90%, about 91%, about 92%, about 93%, about 94%, about 95%, about 96%, about 97%, about 98%, about 99% or 100% identical to a nucleic acid sequence described in any of SEQ ID NOs: 1, 2, 4, 5, 7, 8, 10, 12, 14, 16, 18, 20, 22, and 24. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

Nucleic Acid Constructs

In some embodiments of the present technology, a sequence that confers or increases salt tolerance in a plant is incorporated into a nucleic acid construct that is suitable for introducing into a plant or cell. Thus, such a nucleic acid construct can be used to express or overexpress EsCBL10a, EsCBL10b, EsCBL10 chimeric protein, EsCBL10a major or minor element, and/or EsSOS3 in a plant or cell.

Recombinant nucleic acid constructs may be made using standard techniques. For example, the DNA sequence for transcription may be obtained by treating a vector containing the sequence with restriction enzymes to cut out the appropriate segment. The DNA sequence for transcription may also be generated by annealing and ligating synthetic oligonucleotides or by using synthetic oligonucleotides in a polymerase chain reaction (PCR) to give suitable restriction sites at each end. The DNA sequence then is cloned into a vector containing suitable regulatory elements, such as upstream promoter and downstream terminator sequences.

In some embodiments of the present technology, nucleic acid constructs comprise a sequence encoding an EsCBL10a, EsCBL10b, EsCBL10 chimeric protein, EsCBL10a major or minor element, and/or EsSOS3 operably linked to one or more regulatory or control sequences, which drive expression of the protein-encoding sequence in certain cell types, organs, or tissues without unduly affecting normal plant development or physiology.

Promoters useful for expression of the nucleic acid sequences of the present technology may be constitutive promoters, tissue-specific, tissue-preferred, cell type-specific, or inducible. Examples of promoters include, but are not limited to, a CaMV 35S promoter, a CaMV 19S promoter, a viral coat protein promoter, a monocot promoter, a ubiquitin promoter, an actin promoter, a cab promoter, a sucrose synthase promoter, a tubulin promoter, a napin R gene complex promoter, a tomato E8 promoter, a patatin promoter, a mannopine synthase promoter, a soybean seed protein glycinin promoter, a soybean vegetative storage protein promoter, a bacteriophage SP6 promoter, a bacteriophage T3 promoter, a bacteriophage T7 promoter, a Ptac promoter, a root-cell promoter, an ABA-inducible promoter, and a turgor-inducible promoter.

The vectors of the technology may also contain termination sequences, which are positioned downstream of the nucleic acid molecules of the present technology, such that transcription of mRNA is terminated, and polyA sequences added. Exemplary terminators include Agrobacterium tumefaciens nopaline synthase terminator (Tnos), Agrobacterium tumefaciens mannopine synthase terminator (Tmas), and the CaMV 35S terminator (T35S). Termination regions include the pea ribulose bisphosphate carboxylase small subunit termination region (TrbcS) or the Tnos termination region. The expression vector also may contain enhancers, start codons, splicing signal sequences, and targeting sequences.

Expression vectors of the present technology may also contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

Examples of suitable selectable markers include but are not limited to adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3′-O-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium glufosinate. See, e.g., Thompson et al., EMBO J. 9:2519-23 (1987)). Other suitable selection markers known in the art may also be used.

Visible markers such as green florescent protein (GFP) may be used. Methods for identifying or selecting transformed plants based on the control of cell division have also been described. See, e.g., WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For example, when Agrobacterium is the host, T-DNA sequences may be included to facilitate the subsequent transfer to and incorporation into plant chromosomes.

Such gene constructs may suitably be screened for activity by transformation into a host plant via Agrobacterium.

Suitably, the nucleotide sequences for the genes may be extracted from the GenBank™ nucleotide database and searched for restriction enzymes that do not cut. These restriction sites may be added to the genes by conventional methods such as incorporating these sites in PCR primers or by sub-cloning.

Constructs may be comprised within a vector, such as an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors, A Laboratory Manual, Elsevier, Amsterdam (1986). Examples of suitable vectors include plant binary vectors such as the pEZT-NL vector.

In some embodiments, the present technology provides expression vectors that enable the expression or overexpression of EsCBL10a, EsCBL10b, EsCBL10 chimeric protein, and/or EsCBL10a major and/or minor elements for increasing or conferring salt tolerance in plants. In some embodiments, the expression vectors of the present technology further enable the expression or overexpression of EsSOS3. These expression vectors can be transiently introduced into host plant cells or stably integrated into the genomes of host plant cells to generate transgenic plants by various methods known to persons skilled in the art. When these expression vectors are stably integrated into the genomes of host plant cells to generate stable cell lines or transgenic plants, the expression or overexpression of EsCBL10a, EsCBL10b, EsCBL10 chimeric protein, and/or EsCBL10a major and/or minor elements alone or in combination with EsSOS3, can be deployed as a method for increasing or conferring salt tolerance in a plant.

In some embodiments, an expression vector comprises a promoter operably linked to the cDNA encoding EsCBL10a, EsCBL10b, EsCBL10 chimeric protein, and/or EsCBL10a major and/or minor elements. In another embodiment, a plant cell line comprises an expression vector comprising a promoter operably linked to the cDNA encoding EsCBL10a, EsCBL10b, EsCBL10 chimeric protein, and/or EsCBL10a major and/or minor elements. In another embodiment, a transgenic plant comprises an expression vector comprising a promoter operably linked to the cDNA encoding EsCBL10a, EsCBL10b, EsCBL10 chimeric protein, and/or EsCBL10a major and/or minor elements. In some embodiments, the expression vector further comprises a promoter operably linked to cDNA encoding EsSOS3.

In another embodiment, an expression vector comprises (i) a first promoter operably linked to cDNA encoding EsCBL10a, and (ii) a second promoter operably linked to cDNA encoding EsCBL10b. In another embodiment, a plant cell line comprises (i) an expression vector comprising a first promoter operably linked to cDNA encoding EsCBL10a, and (ii) a second promoter operably linked to cDNA encoding EsCLB10b. In another embodiment, a transgenic plant comprises (i) an expression vector comprising a first promoter operably linked to cDNA encoding EsCLB10a, and (ii) a second promoter operably linked to cDNA encoding EsCBL10b. In another embodiment, the transgenic plants of the present technology are generated by crossing a transgenic plant engineered to express EsCBL10a with a transgenic plant engineered to express EsCBL10b to produce a transgenic plant expressing EsCBL10a and EsCBL10b in combination.

In another embodiment, an expression vector comprises (i) a first promoter operably linked to cDNA encoding EsCBL10a, and (ii) a second promoter operably linked to cDNA encoding EsSOS3. In another embodiment, a plant cell line comprises (i) an expression vector comprising a first promoter operably linked to cDNA encoding EsCBL10a, and (ii) a second promoter operably linked to cDNA encoding EsSOS3. In another embodiment, a transgenic plant comprises (i) an expression vector comprising a first promoter operably linked to cDNA encoding EsCLB10a, and (ii) a second promoter operably linked to cDNA encoding EsSOS3. In another embodiment, the transgenic plants of the present technology are generated by crossing a transgenic plant engineered to express EsCBL10a with a transgenic plant engineered to express EsSOS3 to produce a transgenic plant expressing EsCBL10a and EsSOS3 in combination.

Host Plants and Cells

In some embodiments, the present technology relates to the genetic manipulation of a plant or cell via introducing a polynucleotide sequence that encodes an EsCBL10a protein, EsCBL10b protein, EsCBL10 chimeric protein, and/or EsCBL10a major and/or minor element protein that confers salt tolerance in a plant. Accordingly, the present technology provides methodology and constructs for increasing salt tolerance in a plant.

The plants utilized in the present technology include higher plants amenable to genetic engineering techniques, including both monocotyledonous and dicotyledonous plants, as well as gymnosperms. In some embodiments, the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

As known in the art, there are a number of ways by which genes and gene constructs can be introduced into plants, and a combination of plant transformation and tissue culture techniques have been successfully integrated into effective strategies for creating transgenic crop plants.

These methods, which can be used in the present technology, have been described elsewhere (Potrykus, Annu. Rev. Plant Physiol. Plant Mol. Biol. 42:205-225 (1991); Vasil, Plant Mol. Biol. 5:925-937 (1994); Walden & Wingender, Trends Biotechnol. 13:324-331 (1995); Songstad, et al., Plant Cell, Tissue and Organ Culture 40:1-15 (1995)), and are well known to persons skilled in the art. For example, one skilled in the art will certainly be aware that, in addition to Agrobacterium-mediated transformation of Arabidopsis by vacuum infiltration (Bechtold, et al., C. R. Acad. Sci. Ser. III Sci. Vie, 316:1194-1199 (1993)) or wound inoculation (Katavic, et al., Mol. Gen. Genet. 245:363-370 (1994)), it is equally possible to transform other plant and crop species, using Agrobacterium Ti-plasmid-mediated transformation (e.g., hypocotyl (DeBlock, et al., Plant Physiol. 91:694-701 (1989)), the floral dip method (Clough & Bent, Plant J. 16(6):735-743 (1998)), or cotyledonary petiole (Moloney, et al., Plant Cell Rep. 8:238-242 (1989) wound infection), particle bombardment/biolistic methods (Sanford, et al., J. Part. Sci. Technol. 5:27-37 (1987); Nehra, et al., Plant 5: 285-297 (1994); Becker, et al., Plant J. 5: 299-307 (1994)), or polyethylene glycol-assisted protoplast transformation (Rhodes, et al., Science 240: 204-207 (1988); Shimamoto, et al., Nature 335: 274-276 (1989)) methods.

Additionally, plants may be transformed by Rhizobium, Sinorhizobium, or Mesorhizobium transformation. (Broothaerts, et al., Nature 433: 629-633 (2005)).

After transformation of the plant cells or plant, those plant cells or plants into which the desired DNA has been incorporated may be selected by such methods as antibiotic resistance, herbicide resistance, tolerance to amino-acid analogues, or using phenotypic markers.

Various assays may be used to determine whether the plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

Increased salt tolerance, effected in accordance with the present technology, can be combined with other traits of interest, such as disease resistance, pest resistance, high yield or other traits. For example, a stable genetically engineered transformant that contains a suitable transgene that confers or increases salt tolerance may be employed to introgress a trait into a desirable commercially acceptable genetic background, thereby obtaining a cultivar or variety that combines increased salt tolerance with said desirable background. Alternatively, cells of an increased salt tolerance plant of the present technology may be transformed with nucleic acid constructs conferring other traits of interest.

Constructs according to the present technology may be introduced into any plant cell, using a suitable technique, such as Agrobacterium-mediated transformation, particle bombardment, electroporation, and polyethylene glycol fusion, or cationic lipid-mediated transfection.

In some embodiments, such cells may be genetically engineered with a nucleic acid construct of the present technology with the use of a selectable or visible marker. In other embodiments, such cells may be genetically engineered with a nucleic acid construct of the present technology without the use of a selectable or visible marker and transgenic organisms may be identified by detecting the presence of the introduced construct. The presence of a protein, polypeptide, or nucleic acid molecule in a particular cell can be measured to determine if, for example, a cell has been successfully transformed or transfected. For example, and as routine in the art, the presence of the introduced construct can be detected by PCR or other suitable methods for detecting a specific nucleic acid or polypeptide sequence. Additionally, genetically engineered cells may be identified by recognizing differences in the growth rate or a morphological feature of a transformed cell compared to the growth rate or a morphological feature of a non-transformed cell that is cultured under similar conditions.

B. The Duplication of the CBL10 Calcium Sensor Increased the Complexity of Calcium-Mediated Signaling in Eutrema's Response to Salt.

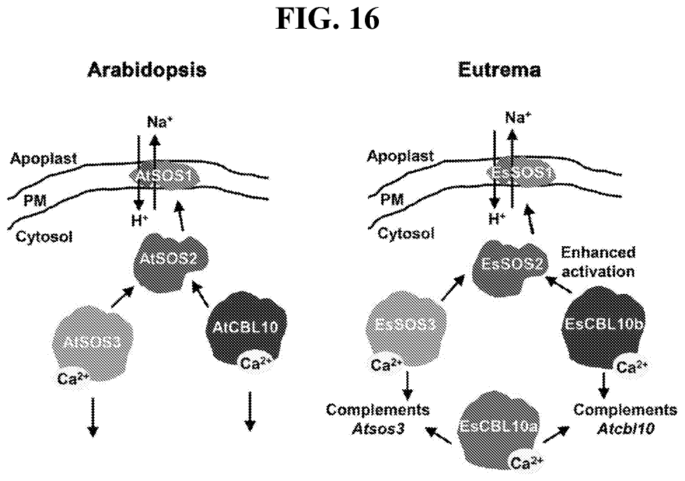

In Arabidopsis, CBL10 and SOS3 function during plant growth in the presence of salt; loss of either gene results in a salt-sensitive phenotype (Liu & Zhu, 1998; Quan, 2007). Both activate AtSOS2 and AtSOS1 in yeast (Quintero, et al., 2002; Quan, 2007) and AtSOS1 activity is reduced in plasma membrane vesicles from the Atsos3 and Atcbl10 mutants (Qiu, et al., 2002; Lin, et al., 2009) suggesting that both function in the SOS pathway (summarized in FIG. 16). However, AtSOS3 and AtCBL10 cannot complement each other's salt-sensitive mutant phenotype even when expressed under the control of the constitutive CaMV 35S promoter suggesting that each protein also has distinct functions (FIG. 16). Previous studies provide evidence for a role for AtCBL10 outside of the SOS pathway. During reproductive development when plants are grown in the presence of NaCl, loss of AtCBL10 but not AtSOS3, AtSOS2 or AtSOS1 resulted in a fertilization defect (Monihan, et al., 2016). In addition, there is evidence for AtCBL10's localization at the tonoplast and regulation of sodium accumulation in the vacuole (Kim, et al., 2007).

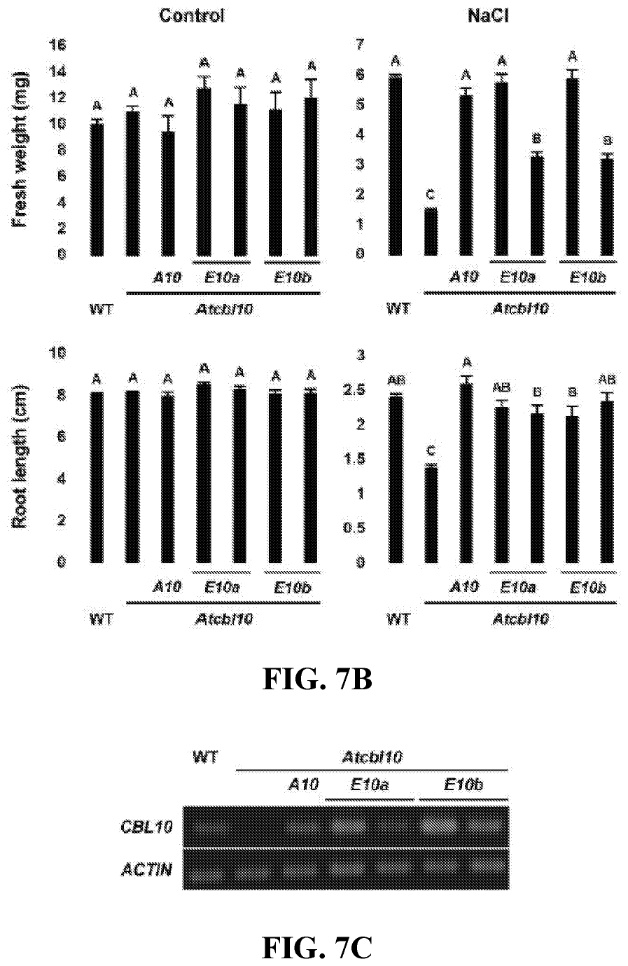

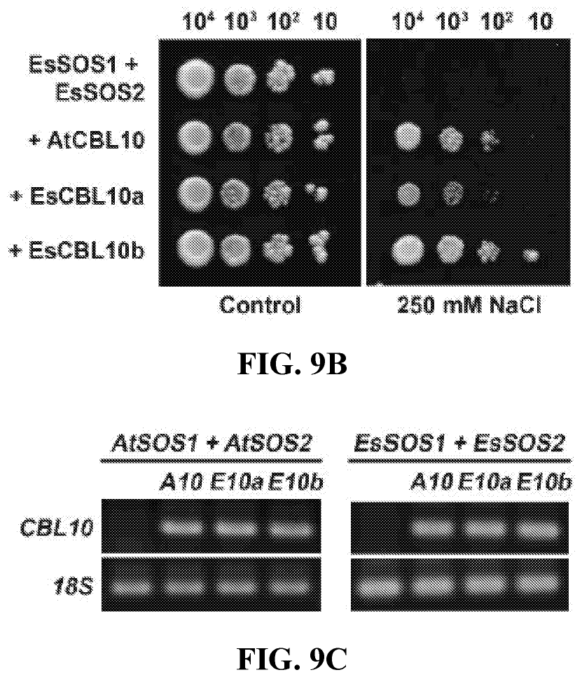

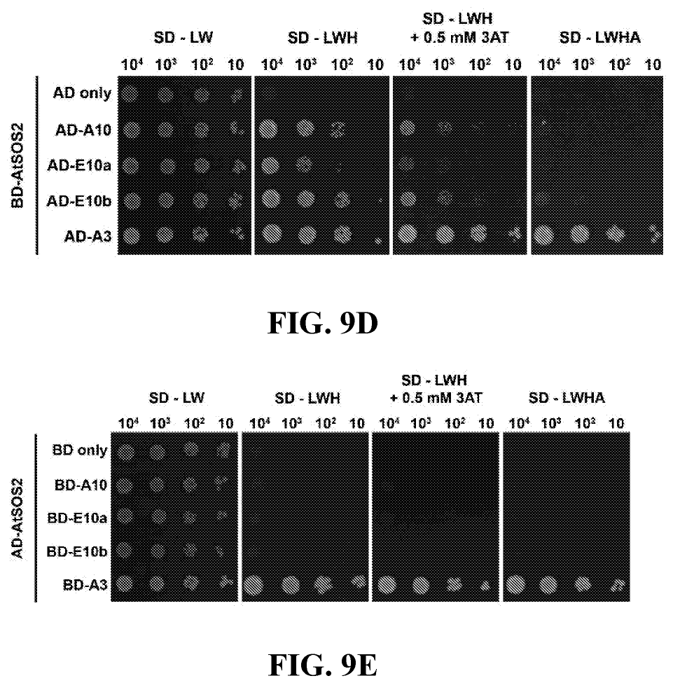

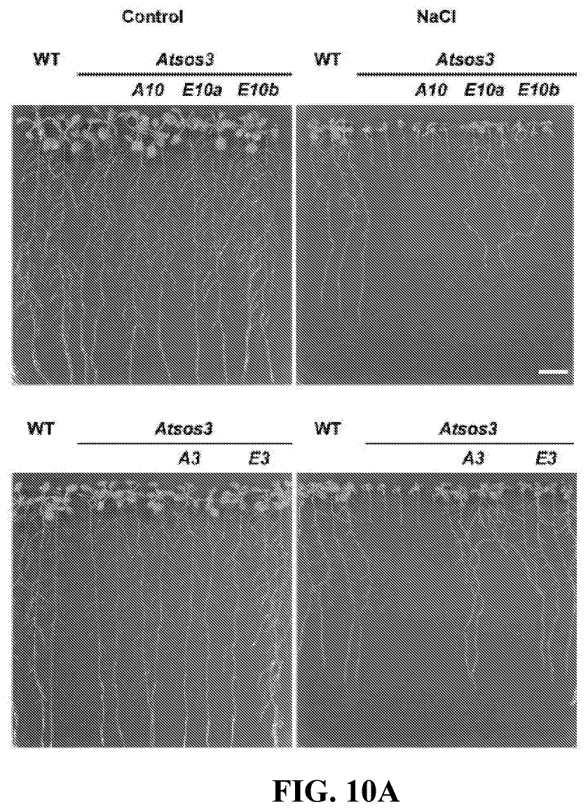

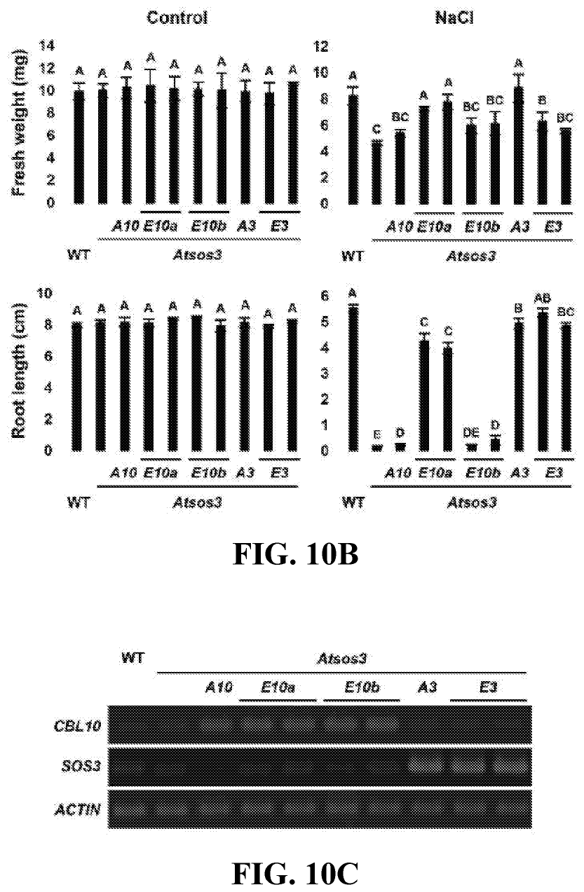

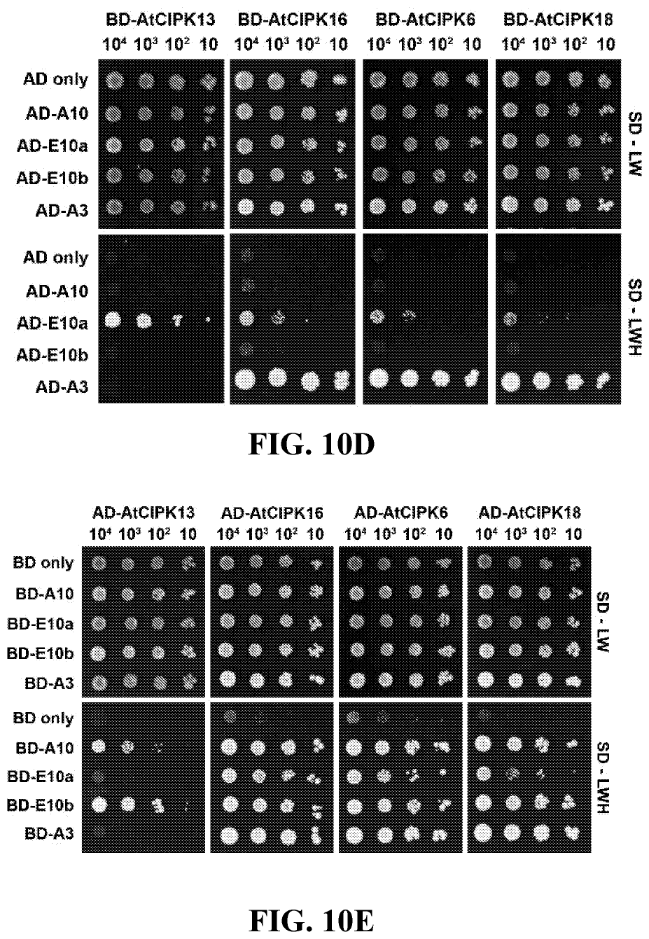

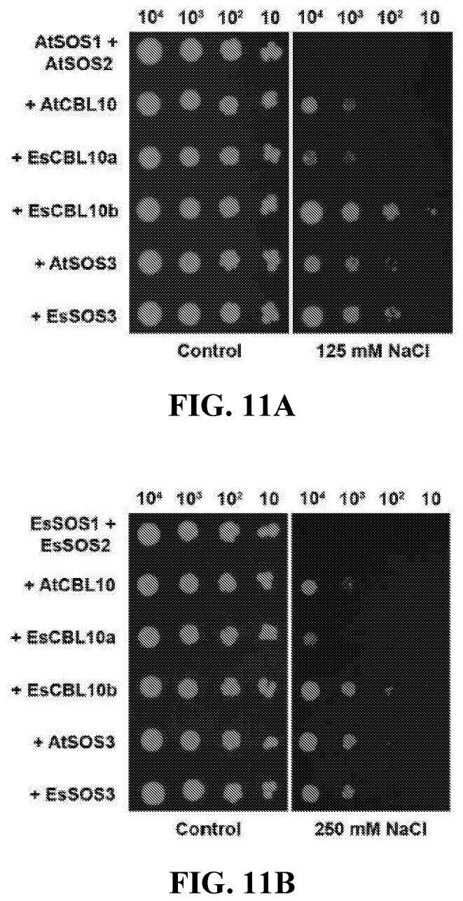

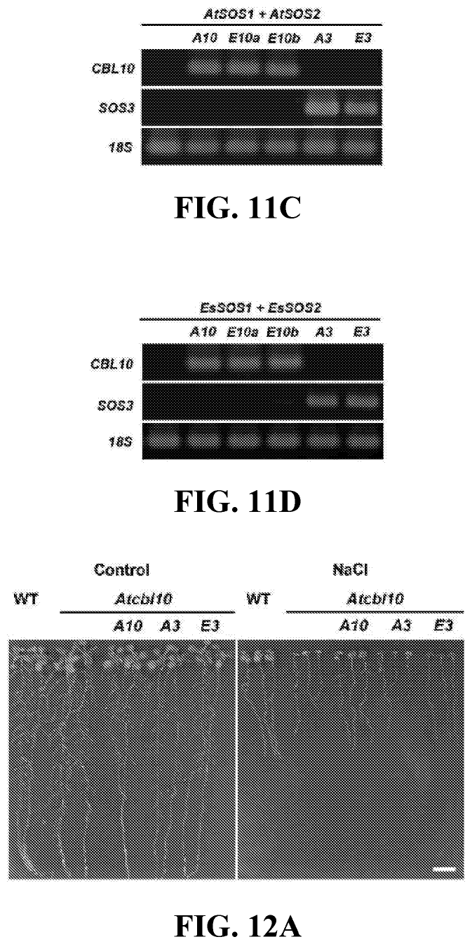

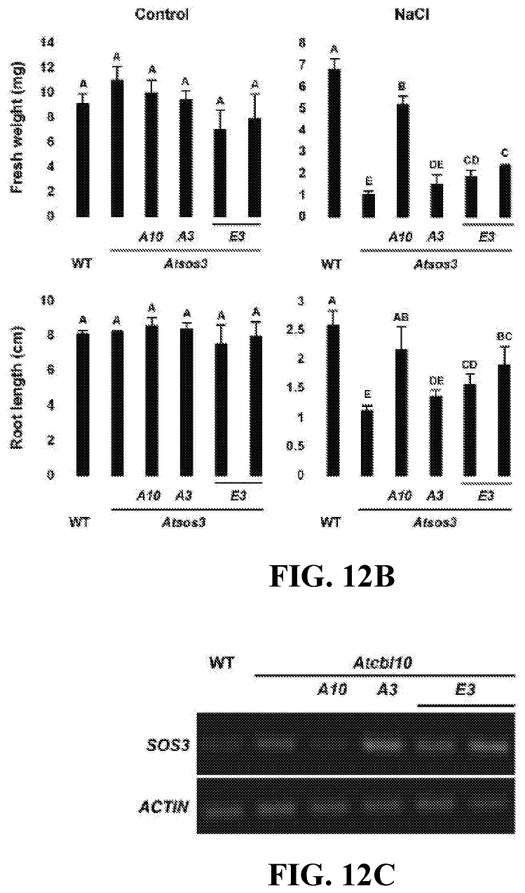

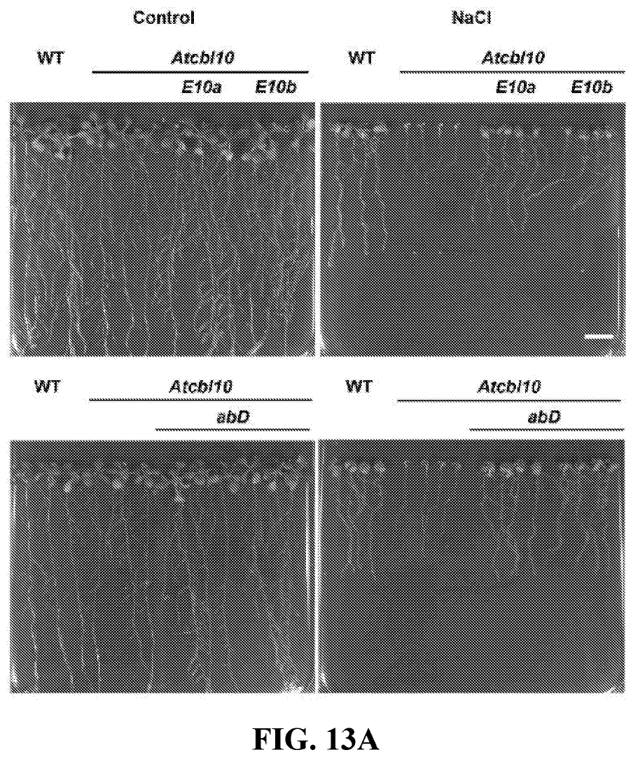

Results provided herein demonstrate that the duplication of CBL10 in Eutrema resulted in three calcium sensors, EsCBL10a, EsCBL10b, and EsSOS3, with both overlapping and distinct roles in seedling responses to salt. EsCBL10a and EsCBL10b complement the Atcbl10 seedling salt-sensitive phenotype and the fertility defect suggesting that they can function in a manner similar to AtCBL10 (FIGS. 7A-7C and FIG. 16). Reduced expression of either gene singly results in a salt-sensitive phenotype and reduced expression of the two genes in combination leads to an even greater sensitivity to salt indicating that EsCBL10a and EsCBL10b have distinct roles. Additional evidence for a divergence in function was seen in differences in the ability of EsCBL10a and EsCBL10b to activate the SOS pathway and complement Atsos3 (FIGS. 9A-9C, 10A-10C, and FIG. 16). EsCBL10a's ability to complement Atsos3 suggested that it might have overlapping functions with EsSOS3 (FIGS. 10A-10C). However, EsCBL10a and EsSOS3 complement Atsos3 in a tissue-specific manner (FIGS. 10A-10C) and have differing abilities to complement Atcbl10 (FIGS. 12A-12C) and activate the SOS pathway (FIGS. 11A-11D) suggesting that these genes have distinct roles in response to salt (FIG. 16).

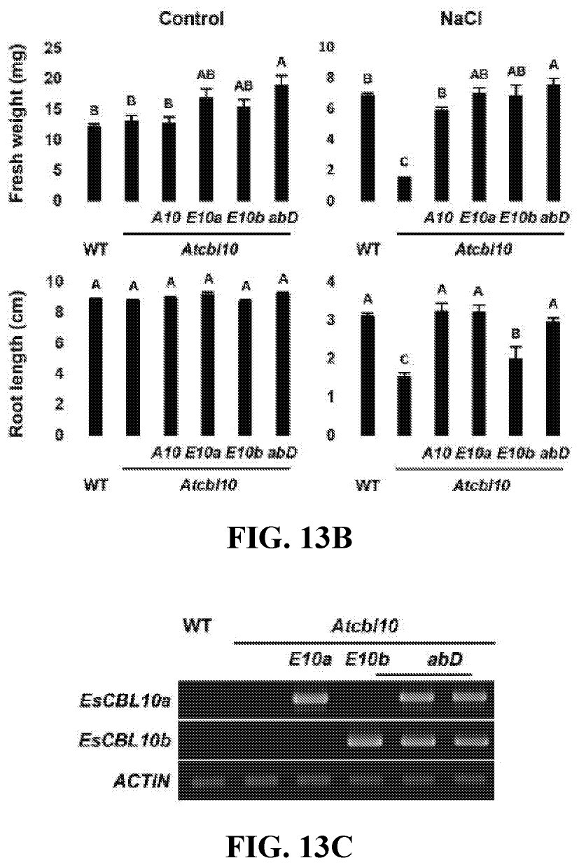

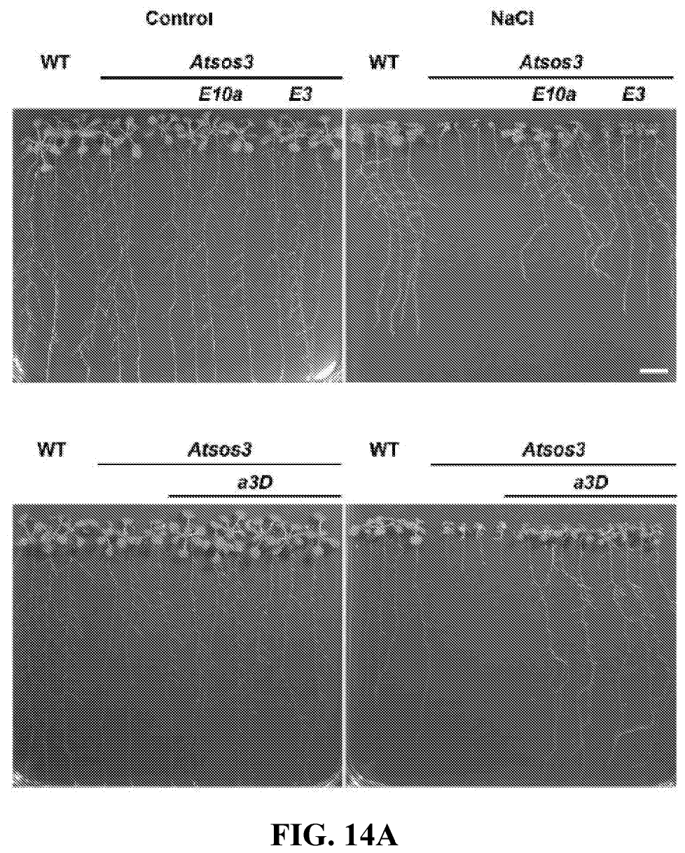

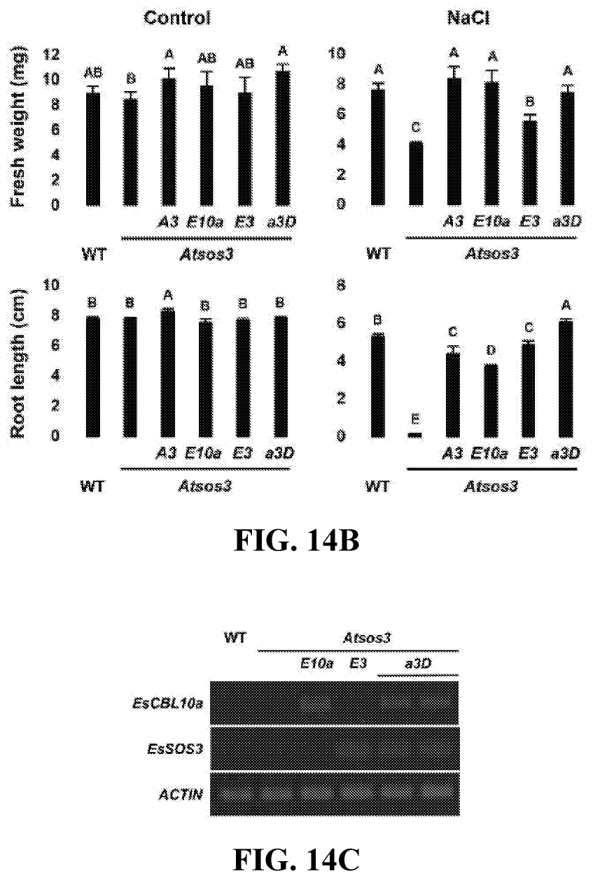

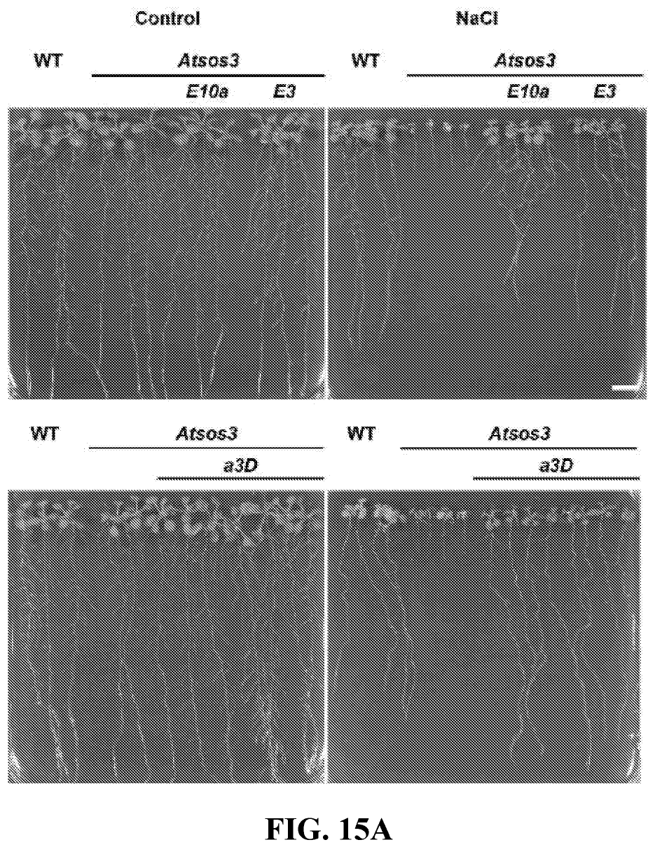

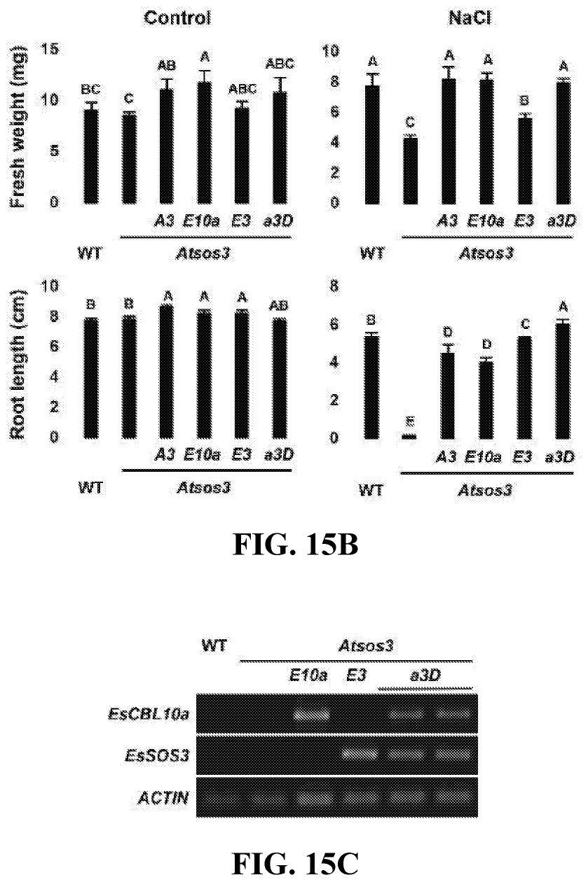

The duplication in Eutrema expanded the complexity of calcium sensors that contribute to its salt tolerance. The expression of EsCBL11a and EsCBL10b in combination in Atclb10 can confer salt tolerance to Arabidopsis (FIGS. 13A-13C). When EsCBL10a and EsSOS3 were expressed together in Atsos3, root growth increased beyond wild type and Atsos3 expressing the genes singly suggesting that the combined functions of EsCBL10a and EsSOS3 are important for root growth in the presence of NaCl and can confer salt tolerance to Arabidopsis (FIGS. 14A-14C and 15A-15C). Longer roots have been shown to be advantageous in salt-affected soils increasing the ability of plants to access water and nitrogen deeper in the soil (Lynch, 2013).

C. Multiple CBL10 Genes have been Observed in Other Plants.

Multiple CBL10 genes have been identified in Populus trichocarpa (Poplar; (Tang, et al., 2014)) and Schrenkiella parvula (Schrenkiella; formerly known as Thellungiella parvula; (Dassanayake, et al., 2011)). CBL10A and CBL10B genes in Poplar complement Atcbl10 suggesting they are functional homologs of AtCBL10 (Tang, et al., 2014). Both interact with PtSOS2 but the proteins were detected at the vacuolar membrane suggesting that they function outside of the SOS pathway (Tang, et al., 2014). While both genes appear to function in plant responses to salt, no difference in the function of the two genes was observed. Currently, little is known about the function of the CBL10 genes in Schrenkiella.

D. Mechanisms Underlying Differences in EsCBL10 Function.

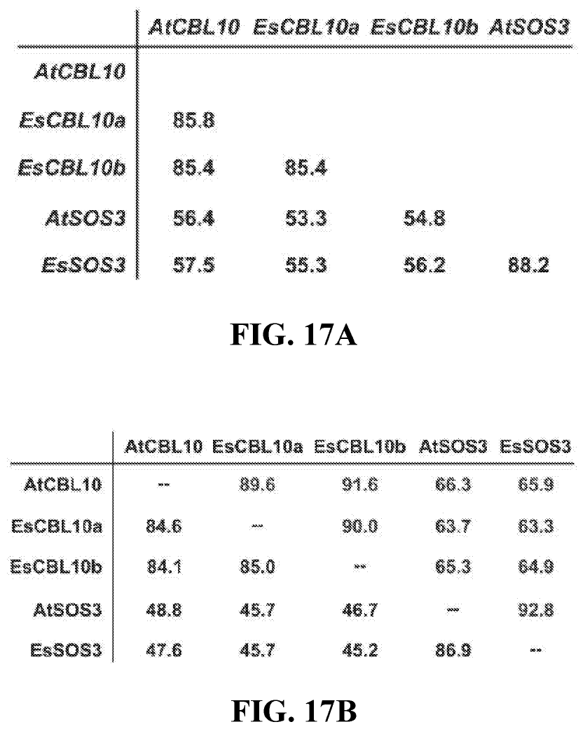

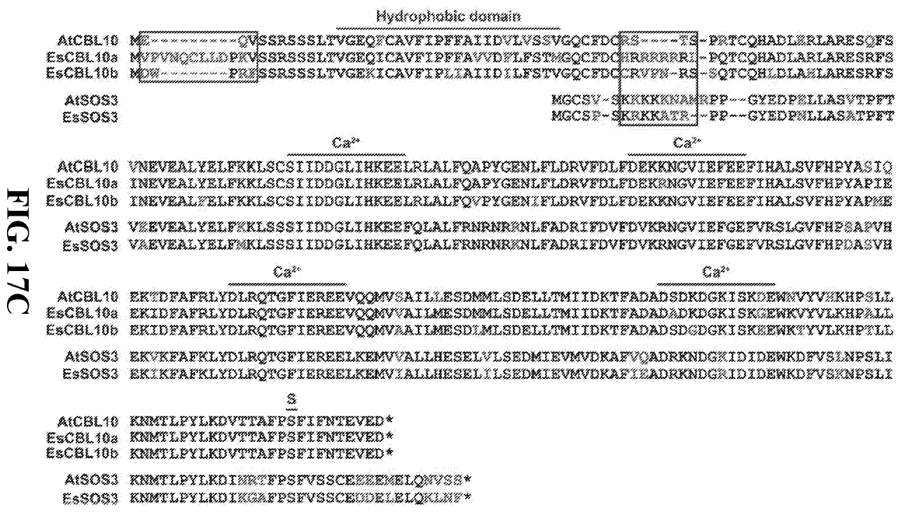

Calcium sensors function by binding calcium, undergoing a conformational change, and interacting with a target protein. Specificity in sensor function depends on the expression and localization of the sensor, its affinity for calcium, and its interaction with a target protein. Several possible mechanisms might underlie the EsCBL10 divergence in function. EsCBL10a's ability to complement Atsos3 (and potentially its EsCBL10b-independent function in Eutrema) might involve a change in protein subcellular localization and/or interacting proteins. EsCBL10b's enhanced ability to activate the Eutrema SOS pathway might be due to a stronger interaction with or activation of SOS2. To provide insight into how two initially redundant genes diverged in function, nucleotide and amino acid sequences of EsCBL10a, EsCBL10b, AtCBL10, EsSOS3, and AtSOS3 were compared. The high level of identity and similarity among the CBL10 sequences suggests that the divergence in function is due to only a few amino acid changes (FIGS. 17A-17B). There are two regions in the CBL10 proteins in which the amino acids vary. At the amino-terminus of the protein, there is an insertion of seven amino acids in EsCBL10a that is not present in EsCBL10b or AtCBL10 and amino acids present in EsCBL10b differ from those in AtCBL10 (FIG. 17C). Adjacent to the hydrophobic domain is a region in which the amino acid sequence varies in all three proteins. EsCBL10a has a series of arginine residues which have similar properties (positively charged side chains) to that of the lysine residues present in the SOS3 proteins (FIG. 17C). Within this region, EsCBL10b has additional and different amino acids compared to AtCBL10 (FIG. 17C). Both of these regions could influence protein localization and/or interaction with a target protein. In order to identify the amino acids underlying the EsCBL10 functions, chimeric proteins fusing regions of the EsCBL10 genes expressed in Atcbl10 to verify that the protein is functional, in Atsos3 to identify regions of EsCBL10a important for complementation of the Atsos3 salt-sensitive phenotype, and in yeast to identify regions of EsCBL10b important for activation of the SOS pathway will be required.

Differences in CBL10 function can be achieved through changes in protein localization, affinity for calcium, interaction with and activation of a target protein, or phosphorylation by a target protein. Without wishing to be bound by theory, it is reasoned that the enhanced ability of EsCBL10b to activate the SOS pathway might be due to a stronger interaction with SOS2 and this hypothesis was tested using yeast two-hybrid assays. EsCBL10b interacted with AtSOS2 more strongly than EsCBL10a, but at a level similar to AtCBL10 (FIG. 9D). This result suggests that interaction alone cannot explain pathway activation and that additional factors likely play a role.

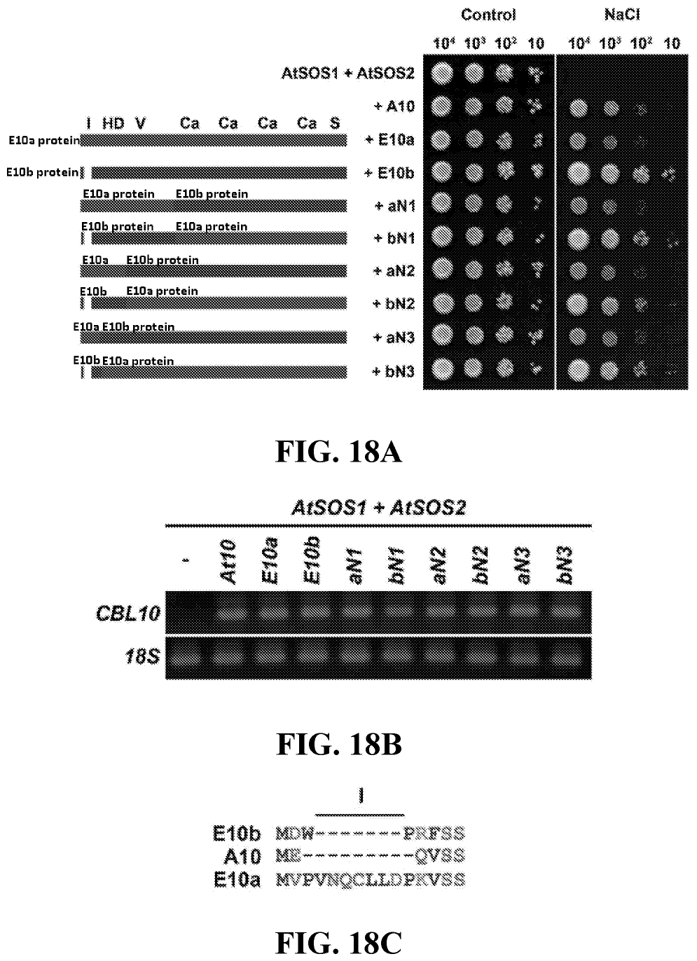

Experiments with chimeric proteins revealed that the ability of EsCBL10b to strongly activate the SOS pathway resides in the first eight amino acids of the protein (FIGS. 18A-18C). The AtCBL10 hydrophobic domain is large enough to span the membrane (Quan et al., 2007); however, this would mean that the amino acids that underlie EsCBL10b's activation of the SOS pathway would be extracellular. If the EsCBL10b hydrophobic domain associates with the membrane via a hydrophobic loop (Winter et al., 2009; Hernandez-Gras and Boronat, 2015; Xu et al., 2016), the amino acids 5′ to the hydrophobic domain would reside in the cytosol and might influence subcellular localization and/or interaction with a target protein. In EsCBL10a, seven amino acids are present in this region, suggesting that the reduced ability of EsCBL10a to activate the SOS pathway might be due to this insertion (FIG. 18C).

Because EsCBL10a only weakly activated the SOS pathway and showed little interaction with SOS2, yeast two-hybrid assays were performed to determine if interaction with a different kinase underlies EsCBL10a's ability to complement Atsos3. Four CIPK proteins were identified that interact with EsCBL10a but not EsCBL10b or AtCBL10 (FIG. 10D). Three of the four also interact with AtSOS3 suggesting that EsCBL10a might be performing both SOS3-like and SOS3-independent functions (FIG. 10D). Two of the identified CIPK proteins, AtCIPK16 and AtCIPK6, have known roles in salt tolerance (Tsou et al., 2012; Roy et al., 2013); both have been shown to interact with and activate the potassium transporter, AtAKT1 (Lee et al., 2007). In addition, AtCIPK6 has been shown to interact with AtSOS3 to recruit the potassium transporter, AtAKT2, to the plasma membrane (Held et al., 2011). These results suggest that EsCBL10a could interact with AtCIPK6 and/or AtCIPK16 proteins to regulate potassium levels in Atsos3, thereby alleviating some of the toxic effects of sodium. A homolog of AtCIPK16 has been identified in Eutrema but the role of EsCIPK16 in plant responses to salt has not been explored (Amarasinghe et al., 2016). Future studies focusing on EsCBL10a interaction with EsCIPK proteins and down-regulation of EsCIPK gene expression will link the associated proteins to salt tolerance. EsCBL10a also interacted with AtCIPK13 and AtCIPK18 (FIG. 10D); however, a role for these genes in salt tolerance has not been reported. Single and double-mutant phenotypes (based on the phylogenetic relatedness of the two genes) were assessed; however, salt-sensitive phenotypes were not detected (data not shown). These results suggest that these genes do not function with EsCBL10a to complement Atsos3 or that additional redundant genes prevent the manifestation of a salt-sensitive phenotype.

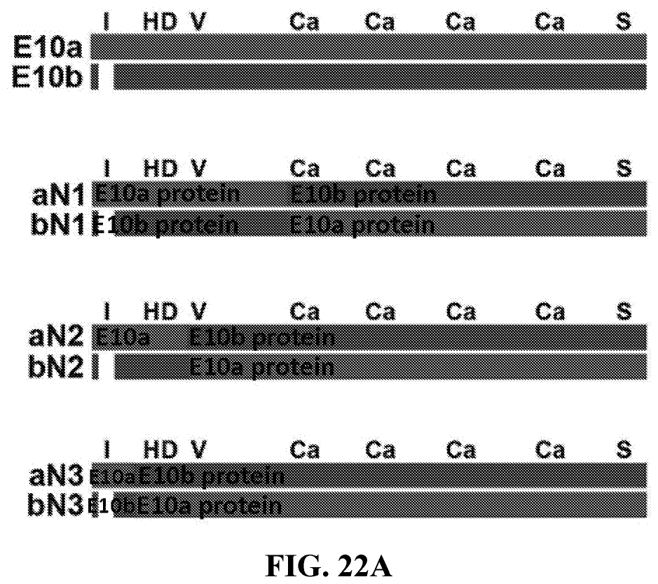

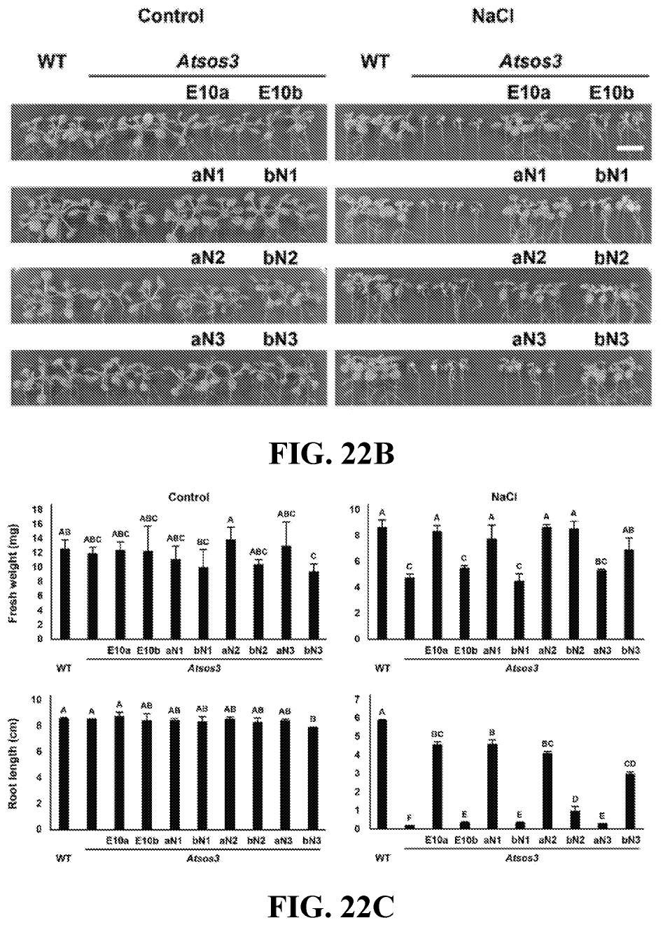

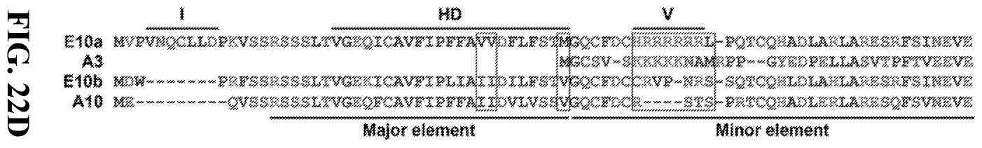

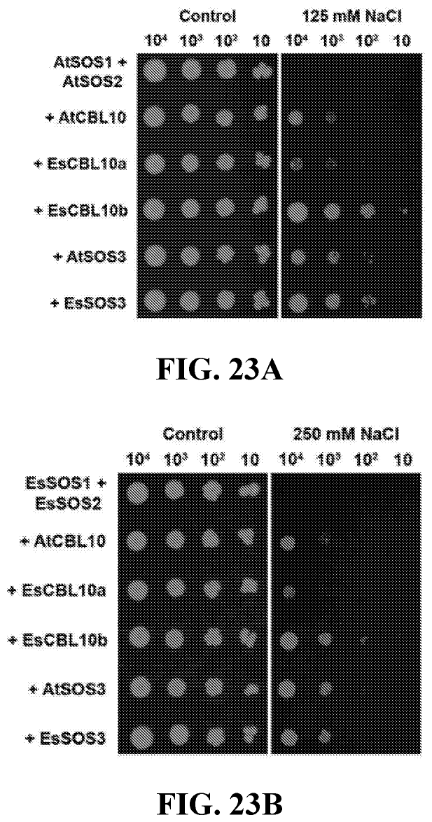

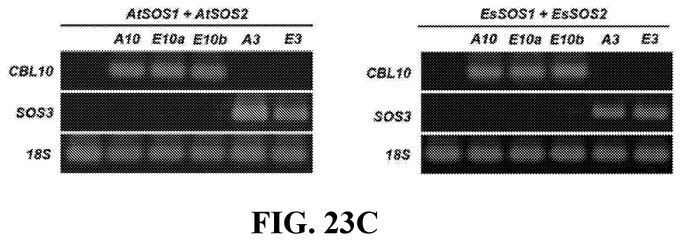

EsCBL10 chimeric proteins revealed that the EsCBL10a-specific function resides in the N-terminus within the hydrophobic domain. While little is known about the function of the hydrophobic domain in EsCBL10, studies in Arabidopsis and Poplar (Populus trichocarpa) suggest that the CBL10 hydrophobic domain is important for subcellular localization. Removal of the domain from AtCBL10 prevented it from recruiting AtSOS2 to the plasma membrane (Quan et al., 2007), while its removal from the duplicated CBL10 proteins in Poplar prevented their localization to the vacuolar membrane (Tang et al., 2014). When compared to AtCBL10 and EsCBL10b, two differences in amino acid sequence were observed in the hydrophobic domain of EsCBL10a (major element; FIGS. 22A-22D). A second element was identified in EsCBL10a that confers partial function (minor element; FIGS. 22A-22D). The chimeric protein containing the EsCBL10b hydrophobic domain and the EsCBL10a variable domain was able to restore rosette growth to wild-type like levels but not root length (bN2; FIGS. 22A-22D). Because of its proximity to the hydrophobic domain this region might influence localization of the protein. Alternatively, while the three-dimensional structure of other CBL proteins indicates that this region would be unlikely to directly interact with a protein partner, it might regulate initial recognition of that protein.

E. Changes in CBL10 Expression May have Also Increased the Complexity of Calcium-Mediated Signaling in Eutrema's Response to Salt.

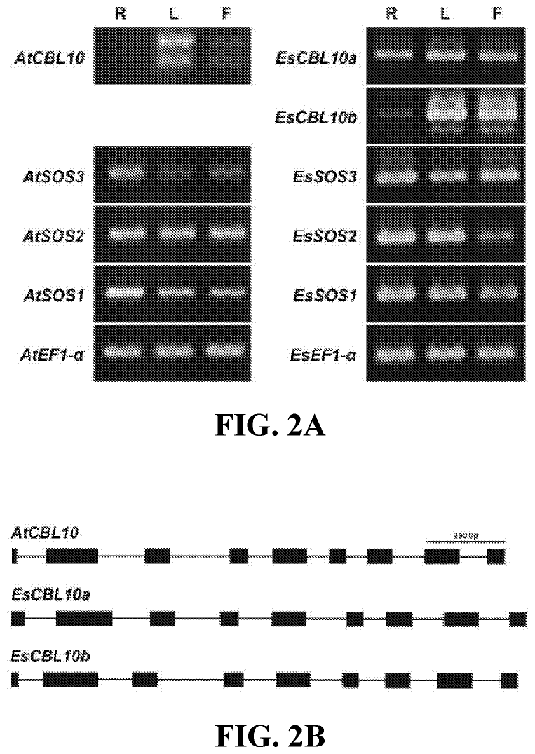

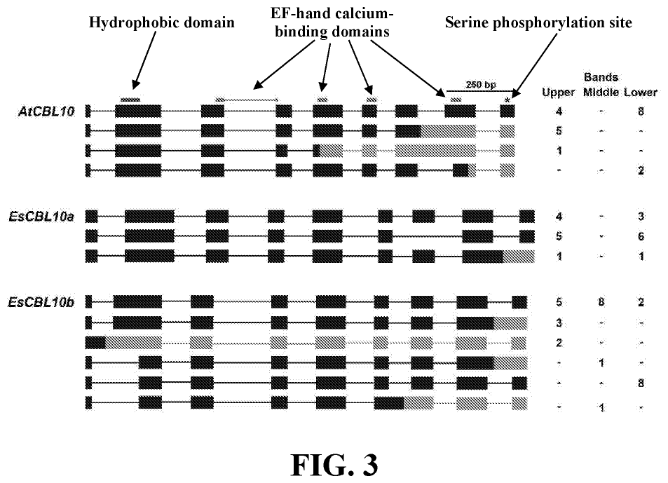

The results presented herein indicate that the expression patterns of the EsCBL10 genes diverged; the EsCBL10b transcript is predominantly present in aerial tissues while the EsCBL10a transcript is present throughout the plant FIG. 2A). Alternative splicing of AtCBL10 results in two major variants; only one of which encodes a functional protein (Quan et al., 2007). Most of the EsCBL10a and EsCBL10b splice variants identified likely encode non-functional proteins due to premature stop codons or the absence of domains required for binding calcium (FIG. 3). However, one EsCBL10b variant, abundant in the lower band in the RT-PCR gel (FIG. 2A), lacks the hydrophobic domain (thought to target AtCBL10 to the plasma membrane; (Quan et al., 2007)), but has all four calcium-binding domains suggesting that it may encode a functional protein with a different subcellular localization (FIG. 3). Once functional variants have been identified, it may be possible to determine if transcriptional regulation of the EsCBL10 genes further expanded calcium signaling in Eutrema in response to salt by linking transcript presence and abundance to function.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Materials and Methods

Plant material. Arabidopsis thaliana Columbia-0 (Arabidopsis) and Eutrema salsugineum Shangdong (Eutrema) were used as wild type for this study. An Arabidopsis CBL10 T-DNA insertion line (SALK 056042) was obtained from the Arabidopsis Biological Resource Center and a homozygous mutant was generated (Monihan, et al., 2016). The Arabidopsis sos1-1 (Wu, et al., 1996) sos2-2 (Zhu, et al., 1998), and sos3-1 (Liu & Zhu, 1997) ethyl methanesulfonate mutants were obtained from Dr. Jian-Kang Zhu (Purdue University).

Plant growth and salt assays. To monitor seedling shoot and root development, Arabidopsis seeds were sown on 0.5× Murashige and Skoog (MS) media (PhytoTechnology Laboratories) containing 2.5 mM 2-(N-morpholino)ethanesulfonic acid (EMS), 2% sucrose, and 1% agar (A8678; Sigma). Eutrema seeds were sown on Eutrema Basal Media (EBM) containing 3 mM calcium nitrate, 1.5 mM magnesium sulfate, 1.25 mM potassium phosphate, 1× micronutrient solution (100 μM boric acid, 0.1 μM cobalt chloride, 0.1 μM cupric sulfate, 100 μM ferrous sulfate, 100 μM manganese sulfate, 5 μM potassium iodine, 1 μM sodium molybdate, 30 μM zinc sulfate, 100 μM disodium EDTA), 2.5 mM MES, 2% sucrose, and 1% agar. All media was brought to a final pH of 5.7 (adjusted with potassium hydroxide). Plates with seed were incubated at 4° C. in the dark for 2 (Arabidopsis) or 4 (Eutrema) days to break dormancy and transferred to a growth chamber at 21° C. under a 16 hour light/8 hour dark photoperiod with light provided by Phillips F32T8/TL841 bulbs (135 μmol m⁻² s⁻¹).

For salt assays, seeds were germinated on media without sodium chloride (NaCl) for 4 (Arabidopsis) or 5 (Eutrema) days after which seedlings were transferred to media without or with the indicated concentration of NaCl. NaCl concentrations were chosen for maximal differences in growth between wild type and mutants or knockdown lines. Values for fresh weight represent the entire seedling weight; values for root length represent the amount of growth from the time of seedling transfer to the end of the assay, 10 days for Arabidopsis and 14 days for Eutrema.

To monitor vegetative (rosette) and reproductive development, Arabidopsis and Eutrema were grown in SunGro Sunshine LC1 soil mix (SunGrow Horticulture) and watered every 3 days with 0.5× Hoagland's solution (Hoagland & Arnon, 1938) with cobalt chloride in place of cobalt nitrate and a final pH of 5.7 (adjusted with potassium hydroxide). To break dormancy, seeds were stratified for 4 days at 4° C. in the dark. To monitor reproductive development, seeds were stratified for 3 weeks at 4° C. in the dark to break dormancy and promote flowering in Eutrema without vernalization. After cold treatment, plants were transferred to a growth chamber at 21° C. under a 16 hour light/8 hour dark photoperiod with light provided by Phillips F32T8/TL841 bulbs (135 μmol m⁻² s⁻¹). For salt assays, NaCl was added to 0.5× Hoagland's solution and plants were treated with NaCl in 50 mM increments every 3 days until the indicated, final concentration was reached. To monitor the effect of salt on development, NaCl treatments were started 1 week after germination and continued for 3 weeks (vegetative development) or started 3 weeks after germination, at the start of inflorescence development, and continued for 4 weeks (reproductive development).

Transcript analysis. Arabidopsis and Eutrema leaves and roots of 2-week-old seedlings grown on MS or EBM plates and open flowers (stage 14, (Smyth, et al., 1990)) from plants grown in soil were collected for transcript analysis. To determine transgenic transcript levels in Arabidopsis, 2-week-old seedlings grown on MS plates were collected. RNA was isolated using the Qiagen RNeasy Plant Mini Kit, treated with TURBO DNase (Invitrogen), purified (RNeasy MinElute Cleanup Kit; Qiagen), and used to synthesize cDNA (M-MLV Reverse Transcriptase; Promega). All PCR amplifications were performed using Recombinant Taq Polymerase (Invitrogen) and primers are provided in Tables 1 and 2. Multiple bands (2-3) were identified for the endogenous CBL10 genes. Products from each band were cloned into pGEM-T Easy (Promega) and ten clones were sequenced per band to identify alternatively spliced variants.

TABLE 1
Primers.

| | | |
|---|---|---|
| Transcript | Forward primer | Reverse primer |

Analysis of endogenous transcript levels in Arabidopsis and Eutrema

| | | |
|---|---|---|
| AtCBL10 | TGAGCTATTCAAGAAATTGAGCTG | ACTAATTTTACCGTCTTTGTCAGAA |
| AtSOS3 | GCGCTCGACATGGGCTGGGCTGCTCTGTA | GGCGAATTCGGAAGATACGTTTTGCAATTCC |
| | TCGAAG | |
| AtSOS2 | GGTTTTCCGCAGAAGTGAAG | ATTTACGAGGTGGCACCATC |
| ALSOS1 | AGTGATTCTCTCGTTCTTGT | AAATTGGGTAGTGGATCCATTAACTATCAGA |
| AtEF1-α | TGAGCACGCTCTTCTTGCTTT | CCACTGGCACCGTTCCAAT |
| EsCBL10a | GCGCTCGAGATGGTCCCGGTTAATCAATG | GGCGGATCCCGGTCTTCAACCTCTGTGTTGA |
| EsCBL10b | GCGCTCGAGATGGACTGGCCCAGATTTTCC | GGCGGATCCCGGTCTTCAACCTCAGTATTGA |
| EsSOS3 | AGAGGAAGAAGGCAACACG | GTTCTTGCGATCTGCTTCG |
| EsSOS2 | TTACGATGGTTCAGCAGCAG | CCAGCAGCCTTTCTAACGTC |
| EsSOS1 | CAAGCAGCACGATACAGT | CCTCTAGATCACACATCGTTCCTGAA |
| EsEF1-α | TGAGCACGCTCTTCTTGCTTT | CCACTGGCACCGTTCCAAT |

Analysis of transcript levels in amiRNA lines (qRT-PCR)

| | | |
|---|---|---|
| EsCBL10a | GGTTAATCAATGTCTCCTGGAC | TGGAGAAGAGGAAATCAACA |
| EsCBL10b | CCTCTAGATCCAGTTCTTTG | GACGATCGATTGGGGACAC |
| ACTIN2 | CAAGCAGCATGAAGATTAAGGTCGTT | CTTGGAGATCCACATCTGCTGGAAT |

Cloning for expression in Atcbl10 and Atsos3

| | | |
|---|---|---|
| AtCBL10 | GCGCTCGAGATGGAACAAGTTTCCTCTAGAT | GGCGGATCCCGGTCTTCAACCTCAGTGTTGAA |
| EsCBL10a | GCGCTCGAGATGGTCCCGGTTAATCAATG | GGCGGATCCCGGTCTTCAACCTCTGTGTTGA |
| EsCBL10b | GCGCTCGAGATGGACTGGCCCAGATTTTCC | GGCGGATCCCGGTCTTCAACCTCAGTATTGA |
| EsSOS3 | GCGCTCGAGATGGGCTGCTCTCCGTCGAAG | GCCGAATTCGAAAATTTAGTTTTTGCAACTCC |
| | | AATTCATCG |

Analysis of transcript levels in Atcb110 and Atsos3

| | | |
|---|---|---|
| AtCBL10 | GGCTTGATTCACAAGGAAGA | TCATAGAGCCTAAATGCAAA |
| EsCBL10a | | |
| EsCBL10b | | |
| AtSOS3 | AGAAGTGGAGGCTTTGTATGAACTG | CCACCATTACTTCAATCATATCTTCGG |
| EsSOS3 | | |
| ACTINS2 | GTCGTACAACCGGTATTGTG | GAGCTGGTCTTTGAGGTTTC |
| EsCBL10a | GGTTAATCAATGTCTCCTGGAC | TGGGAATGCTGTTGTCACATCC |
| EsCBL10b | CCTCTAGATCCAGTTCTTTG | TGGGAATGCTGTTGTCACATCC |
| EsSOS3 | AGAGGAAGAAGGCAACACG | GTTCTTGCGATCTTCGTTCG |

Cloning for expression in yeast

| | | |
|---|---|---|
| AtCBL10 | GCGCTCGAGATGGAACAAGTTTCCTCTAGAT | GCGGCGGCCGCTCAGTCTTCAACCTCAGTGTTG |
| EsCBL10a | GCGCTCGAGATGGTCCCGGTTAATCAATG | TTTGCGGCCGCTCAGTCTTCAACCTCTGTGT |
| EsCBL10b | GCGCTCGAGATGGACTGGCCCAGATTTTCC | TTTGCGGCCGCTCAGTCTTCAACCTCAGTATTG |

Analysis of transcript levels in yeast

| | | |
|---|---|---|
| AtCBL10 | GGCTTGATTCACAAGGAAGA | TCATAGAGCCTAAATGCAAA |
| EsCBL10a | | |
| EsCBL10b | | |
| AtSOS3 | GCTTTGTATGAACTGTTCAAGAA | TTCTCGCTCGATGAATCCAG |
| EsSOS3 | | |
| 18SrRNA | AAACGGCTACCACATCCAAG | CCTCCAATTGTTCCTCGTTA |

TABLE 2
Primers.

| | | |
|---|---|---|
| Transcript | Forward primer | Reverse primer |

Analysis of endogenous transcript levels
in Arabidopsis and Eutrema

| | | |
|---|---|---|
| AtSOS3 | GCGCTCGAGATGGGCTGCTCTGTAT | GGCGAATTCGGGAAGATACGT |
| | CGAAG | TTTGCAATTCC |
| AtSOS1 | AGTGATTCTCTCGTTCTTGT | AAATTGGGTAGTGGATCCATT |
| | | AACTATCAGA |
| AtEF1-α | TGAGCACGCTCTTCTTGCTTT | CCACTGGCACCGTTCCAAT |
| EsCBL10a | GCGCTCGAGATGGTCCCGGTTAATC | GGCGGATCCCGGTCTTCAACC |
| | AATG | TCTGTGTTGA |
| EsCBL10b | GCGCTCGAGATGGACTGGCCCAGAT | GGCGGATCCCGGTCTTCAACC |
| | TTTCC | TCAGTATTGA |
| EsSOS3 | AGAGGAAGAAGGCAACACG | GTTCTTGCGATCTGCTTCG |
| EsSOS2 | TTACGATGGTTCAGCAGCAG | CCAGCAGCCTTTCTAACGTC |
| EsSOS1 | CAAGCAGCACGATACAGT | CCTCTAGATCACACATCGTTCC |
| | | TGAA |
| EsEF1-α | TGAGCACGCTCTTCTTGCTTT | CCACTGGCACCGTTCCAAT |

Analysis of transcript levels in amiRNA lines (qRT-PCR)

| | | |
|---|---|---|
| EsCBL10a | GGTTAATCAATGTCTCCTGGAC | TGGAGAAGAGGAAATCAACA |
| EsCBL10b | CCTCTAGATCCAGTTCTTTG | GACGATCGATTGGGGACAC |
| ACTIN2 | CAAGCAGCATGAAGATTAAGGTCGT | CTTGGAGATCCACATCTGCTG |
| | T | GAAT |

Cloning for expression in Atcbl10 and Atsos3

| | | |
|---|---|---|
| AtCBL10 | GCGCTCGAGATGGAACAAGTTTCCT | GGCGGATCCCGGTCTTCAACC |
| | CTAGAT | TCAGTGTTGAA |
| EsCBL10a | GCGCTCGAGATGGTCCCGGTTAATC | GGCGGATCCCGGTCTTCAACC |
| | AATG | TCTGTGTTGA |
| EsCBL10b | GCGCTCGAGATGGACTGGCCCAGAT | GGCGGATCCCGGTCTTCAACC |
| | TTTCC | TCAGTATTGA |
| EsSOS3 | GCGCTCGAGATGGGCTGCTCTCCGT | GGCGAATTCGAAAATTTAGTT |
| | CGAAG | TTTGCAACTCCAATTCATCG |
| AtCBL10 | GGCTTGATTCACAAGGAAGA | TCATAGAGCCTAAATGCAAA |
| EsCBL10a | | |
| EsCBL10b | | |
| AtSOS3 | AGAAGTGGAGGCTTTGTATGAACTG | CCACCATTACTTCAATCATATC |
| EsSOS3 | | TTCGG |
| ACTIN2 | GTCGTACAACCGGTATTGTG | GAGCTGGTCTTTGAGGTTTC |
| EsCBL10a | GGTTAATCAATGTCTCCTGGAC | TTCTTCAATAAGGCGGGATG |
| EsCBL10b | CCTCTAGATCCAGTTCTTTG | TGGGAATGCTGTTGTCACATCC |
| EsSOS3 | AGAGGAAGAAGGCAACACG | GTTCTTGCGATCTGCTTCG |

Analysis of transcript levels in yeast

| | | |
|---|---|---|
| AtCBL10 | GGCTTGATTCACAAGGAAGA | TCATAGAGCCTAAATGCAAA |
| EsCBL10a | | |
| EsCBL10b | | |
| AtSOS3 | GCTTTGTATGAACTGTTCAAGAA | TTCTCGCTCGATGAATCCAG |
| EsS0S3 | | |
| 18SrRNA | AAACGGCTACCACATCCAAG | CCTCCAATTGTTCCTCGTTA |

Yeast two-hybrid analysis

| | | |
|---|---|---|
| AtCBL10 | GCGCCATGGAACAAGTTTCCTCTAG | GGCGGATCCCGGTCTTCAACC |
| | AT | TCAGTGTTGAA |
| AtSOS3 | GCGCCATGGAGATGGGCTGCTCTGT | GGCGAATTCGGGAAGATACGT |
| | ATCGAAG | TTTGCAATTCC |
| EsCBL10a | CGGGATCCATATGGTCCCGGTTAAT | TTCTCGAGTCAGTCTTCAACCT |
| | CAATG | CTGTGT |
| EsCBL10b | CGGGATCCATATGGACTGGCCCAGA | TTCTCGAGTCAGTCTTCAACCT |
| | TTTTCC | CAGTATTG |
| AtCIPK1 | CCGGAATTCATGGTGAGAAGGCAAG | CGCGGATCCCTAAGTTACTATC |
| | AGGAGG | TCTTGCTCC |
| AtCIPK2 | CCGGAATTCATGGAGAACAAACCAA | CGCGGATCCCTATGATGGTTCT |
| | GTGTAT | TGCTCTCCT |
| AtCIPK3 | CCCCCCGGGTATGTTGATCCCCAAC | CCGCTCGAGTCACTTTGCTGTT |
| | AAAAAAT | TCTTTCTTA |
| AtCIPK4 | CCGGAATTCATGGAATCTCCATATC | CGCGGATCCTCAATTGTGCCAT |
| | CAAAAT | GAGAGCACA |
| AtCIPK5 | CCCCCCGGGTATGGAGGAAGAACG | CCGCTCGAGTCAACAATCCTC |
| | GCGAGTTC | GGAAGAAGTG |
| AtCIPK6 | CCGGAATTCATGGTCGGAGCAAAAC | CGCGGATCCTCAAGCAGGTGT |
| | CGGTGG | AGAGGTCCAG |
| AtCIPK7 | CCGGAATTCATGGAATCACTTCCCC | CGCGGATCCTTACATGATGTC |
| | AGCCGC | ATTGTGCCAT |
| AtCIPK8 | CCCCCCGGGTATGGTGGTAAGGAAG | CCGCTCGAGTCAACGTCTTTTA |
| | GTGGGCA | CTCTTGGCC |
| AtCIPK9 | CCGGAATTCATGAGTGGAAGCAGAA | CGCGGATCCTTATTGCTTTTGT |
| | GGAAGG | TCTTCAGCG |
| AtCIPK10 | CCGGAATTCATGGAAAATAAGCCAA | CGCGGATCCTCAAAACTTCAA |
| | GTGTTT | TGGTTCTTCC |
| AtCIPK11 | CCGGAATTCATGCCAGAGATCGAGA | CGCGGATCCTTAAATAGCCGC |
| | TTGCCG | GTTTGTTGAC |
| AtCIPK12 | CCCCCCGGGTATGGCGGAGAAAATC | CCGCTCGAGCTATTCAGTGTCA |
| | ACGAGAG | GACGGCAAG |
| AtCIPK13 | CCGGAATTCATGGCTCAAGTACTAT | CGCGGATCCTCACTGTTCAATT |
| | CTACAC | TCAGGTGGC |
| AtCIPK14 | CCCCCCGGGTATGGTAGATTCTGAC | CCGCTCGAGCTACGACGTCGT |
| | CCGGTGG | ATGTACTTGA |
| AtCIPKL5 | CCCCCCGGGTATGGAGAAGAAAGG | CCGCTCGAGTCAGTGCCAAGC |
| | ATCTGTGT | TAATACAAAG |
| AtCIPK16 | CCCCCCGGGTATGGAAGAATCAAAC | CCGCTCGAGTCATGAAACATT |
| | CGTAGTA | ATTTATTTTG |
| AtCIPK17 | CCCCCCGGGTATGGTGATAAAGGGA | CCGCTCGAGCTACGCTAAAAG |
| | ATGCGTG | CTCCTGTACT |
| AtCIPK18 | CCGGAATTCATGGCTCAAGCCTTGG | CGCGGATCCCTATTCAGTATCA |
| | CTCAAC | GATGGCAAA |
| AtCIPK19 | CCGGAATTCATGGCGGATTTGTTAA | CGCGGATCCCTAATCAGTATC |
| | GAAAAG | AGAAAGTAAA |
| AtCIPK20 | CCCCCCGGGTATGGATAAAAACGGC | CCGCTCGAGTTAATGTATCACT |
| | ATAGTTT | TCAATCTTC |
| AtCIPK21 | CCGGAATTCATGGGTTTGTTTGGAA | CGCGGATCCTTAGCTTACTTCC |
| | CGAAGA | GCGGTAAGT |
| AtCIPK22 | CCCCCCGGGTATGTCTTTTACAATTC | CCGCTCGAGTTACGGTTTGTCA |
| | CTAGAC | GGAACTTTA |
| AtCIPK23 | CCCCCCGGGTATGGCTTCTCGAACA | CCGCTCGAGTTATGTCGACTGT |
| | ACGCCTT | TTTGCAATT |
| AtCIPK24 | CCGGAATTCATGACAAAGAAAATGA | CGCGGATCCTCAAAACGTGAT |
| | GAAGAG | TGTTCTGAGA |
| AtCIPK25 | CCCCCCGGGTATGGGATCCAAACTT | CCGGTCGACTTAGCAGTCACT |
| | AAACTTT | ACCAGAATTT |
| AtCIPK26 | CCCCCCGGGTATGAATCGGCCAAAG | CCGCTCGAGTTATTTGCTTAGA |
| | GTTCAGC | CCAGAGCTC |

Chimeric protein generation

| | | |
|---|---|---|
| aN2 | GCGCTCGAGATGGTCCCGGTTAATC | ATCAAAGCACTGTCCCATGGT |
| | AATG | GGAGAAGAGGAAAT |
| bC2 | CTCTTCTCCACCATGGGACAGTGCTT | TTTGCGGCCGCTCAGTCTTCAA |
| | TGATTGCTG | CCTCAGTATTG (yeast) |
| | | GGCGGATCCCGGTCTTCAACC |
| | | TCAGTATTGA (plant) |
| bN2 | GCGCTCGAGATGGACTGGCCCAGAT | ATCGAAGCATTGTCCAACGGT |
| | TTTCC | GGAGAAGAGAATAT |
| bC2 | CTCTTCTCCACCGTTGGACAATGCTT | TTTGCGGCCGCTCAGTCTTCAA |
| | CGATTGCCA | CCTCTGTGT (yeast) |
| | | GGCGGATCCCGGTCTTCAACC |
| | | TCTGTGTTGA (plant) |

Artificial microRNAs (amiRNAs). To reduce expression of EsCBL10a and EsCBL10b singly and in combination, amiRNAs were designed using the Web MicroRNA Designer (WMD) site (wmd3.weigelworld.org, (Schwab, et al., 2006; Ossowski, et al., 2008)). Two 21 base pair sequences per target (EsCBL10a, EsCBL10b, and EsCBL10a/EsCBL10b) were identified. Primers (Table 3) containing the amiRNA sequences were generated and the amiRNA sequence was incorporated into the AtMIR319a precursor gene located on the pRS300 vector (Dr. Detlef Weigel; Max Planck Institute for Developmental Biology; (Ossowski, et al., 2008)). The AtMIR319a gene containing the desired amiRNA sequence was digested with EcoRI-XbaI and subcloned into the corresponding site of the plant binary vector pEZT-NL (Drs. Sean Cutler and David W. Ehrhardt; Carnegie Institution of Washington) behind the Cauliflower Mosaic Virus 35S promoter. Agrobacterium tumefaciens EH105 containing the binary vector was used to transform Eutrema via the floral dip method (Clough & Bent, 1998). T1 seed was germinated on soil for 1.5 weeks and then sprayed 3 times with Basta (18.2% glufosinate ammonium; Rely 200 Herbicide; Bayer Crop Science) at 3-day intervals. T1 lines with antibiotic resistance were subsequently transferred to pots and grown to collect T2 seed. Single insertion lines were identified by screening T2 seed on EBM plates containing 7.5 mg/L glufosinate ammonium (Santa Cruz Biotechnology) and selecting lines with 75% resistance. Homozygous lines were identified by screening T3 seed on EBM plates with glufosinate ammonium and selecting lines with 100% resistance.

TABLE 3
Primers used for generating the amiRNA constructs.

| | | | Primer | |
|---|---|---|---|---|
| Construct | Target | amiRNA sequence | number | Primer sequences |
| a1 | EsCBL10a | TACATTGATTAACGGGGACCA | I | gaTTCACGAGCTAGACGCGCCATtctctcttttgtattcc |
| | | | II | gaATGGCGCGTCTAGCTCGTGAAtcaaagagaatcaatga |
| | | | III | gaATAGCGCGTCTAGGTCGTGATtcacaggtcgtgatatg |
| | | | IV | gaATCACGACCTAGACGCGCTATtctacatatatattcct |
| b1 | EsCBL10b | TACCATCCCCATCCCAGTCCG | I | gaTACCATCCCCATCCCAGTCCGtctctcttttgtattcc |
| | | | II | gaCGGACTGGGATGGGGATGGTAtcaaagagaatcaatga |
| | | | III | gaCGAACTGGGATGGCGATGGTTtcacaggtcgtgatatg |
| | | | IV | gaAACCATCGCCATCCCAGTTCGtctacatatatattcct |
| b2 | EsCBL10b | TTCACGTGCAAGATGAGCCAC | I | gaTTCACGTGCAAGATGAGCCACtctctcttttgtattcc |
| | | | II | gaGTGGCTCATCTTGCACGTGAAtcaaagagaatcaatga |
| | | | III | gaGTAGCTCATCTTGGACGTGATtcacaggtcgtgatatg |
| | | | IV | gaATCACGTCCAAGATGAGCTACtctacatatatattcct |
| ab1 | EsCBL10a | TCATCAAAATAGCAGCCACCA | I | gaTCATCAAAATAGCAGCCACCAtctctcttttgtattcc |
| | and | | II | gaTGGTGGCTGCTATTTTGATGAtcaaagagaatcaatga |
| | EsCBL10b | | III | gaTGATGGCTGCTATATTGATGTtcacaggtcgtgatatg |
| | | | IV | gaACATCAATATAGCAGCCATCAtctacatatatattcct |
| ab2 | EsCBL10a | TAGAAGTTCATCTGACAGCAC | I | gaTAGAAGTTCATCTGACAGCACtctctcttttgtattcc |
| | and | | II | gaGTGCTGTCAGATGAACTTCTAtcaaagagaatcaatga |
| | EsCBL10b | | III | gaGTACTGTCAGATGTACTTCTTtcacaggtcgtgatatg |
| | | | IV | gaAAGAAGTACATCTGACAGTACtctacatatatattcct |

The efficiency of amiRNA targeting was determined using quantitative real-time PCR (qRT-PCR). RNA was isolated from 3-week-old seedlings using TRIZOL (Invitrogen). RNA was treated with RQ1 DNase (Promega), purified using the RNeasy Plant Mini Kit (Qiagen), and used to synthesize cDNA (M-MLV Reverse Transcriptase; Promega). qRT-PCR was performed using the LightCycler Fast Start DNA Master^{PLUS} SYBR Green I kit (Roche) in the LightCycler 1.5 instrument (Roche). Melting-curve analysis was performed to verify that a single PCR product was amplified. C_{T} values were calculated using the LightCycler software 4.0 package (Roche). The relative fold change in expression was determined using the calculation 2^{-ΔΔC}^{_{T}} (Schmittgen & Livak, 2008). All primers are provided in Tables 1 and 2.

Complementation assays. The functions of EsCBL10a, EsCBL10b, and EsSOS3 were analyzed by expressing each gene in the Atcbl10 and Atsos3 mutants. Full-length coding sequences without a stop codon were amplified from cDNA and cloned into pGEM-T Easy (Promega). All PCR amplifications were performed using Phusion High-Fidelity DNA Polymerase (ThermoFisher Scientific); primers are provided in Tables 1 and 2. AtCBL10, EsCBL10a, EsCBL10b were digested with XhoI-BamHI and EsSOS3 was digested with XhoI-EcoRI. All genes were subcloned into the corresponding site of the plant binary vector pEZT-NL containing the Cauliflower Mosaic Virus 35S promoter and a C-terminal fusion to Green Fluorescent Protein. Agrobacterium tumefaciens LBA4404 containing the binary vector was used to transform Atcbl10 and Atsos3 via the floral dip method (Clough & Bent, 1998). A similar strategy was used to generate single insertion, homozygous lines as described above for the amiRNA lines, except MS plates were used in place of EBM plates. AtSOS3 expressed in Atcbl10 and Atsos3 under the control of the Cauliflower Mosaic Virus 35S promoter was provided by Dr. Yan Guo (China Agricultural University). To determine if co-expression of more than one Eutrema calcium sensor confers salt tolerance to Arabidopsis, plants expressing EsCBL10a, EsCBL10b, and EsSOS3 singly were crossed to generate heterozygous plants expressing two genes.

Yeast salt screens. To monitor the ability of EsCBL10a and EsCBL10b to activate the SOS pathway, Saccharomyces cerevisiae strain AXT3K (ena1::HIS3::ena4, nha1::LEU2, and nhx1::KanMX4; (Quintero, et al., 2002)) containing the pYPGE15 plasmid with AtSOS1 or EsSOS1 (Jarvis, et al., 2014) and the pFL32T plasmid with AtSOS2 and AtSOS3 or the pFLE3E2T plasmid with EsSOS2 and EsSOS3 (Jarvis, et al., 2014) was modified to express the CBL10 genes. In order to replace AtSOS3 and EsSOS3 with the CBL10 genes from Arabidopsis and Eutrema, full-length coding sequences of AtCBL10, EsCBL10a, and EsCBL10b were amplified from cDNA (Phusion High-Fidelity DNA polymerase; ThermoFisher Scientific; primers are provided in Table s1 and 2), cloned into pGEM-T Easy (Promega), digested with XhoI-NotI, and subcloned into the corresponding site of the pDR195 vector (Dr. Alonso Rodriguez-Navarro; (Rentsch, et al., 1995)). The plasmid was digested with AgeI-NotI and a fragment containing the CBL10 gene was subcloned into the corresponding site of the pFL32T plasmid in place of AtSOS3 to be expressed with AtSOS2 or the pFLE3E2T plasmid in place of EsSOS3 to be expressed with EsSOS2. Plasmids containing AtSOS2 or EsSOS2 but lacking the CBL10 and SOS3 genes were generated as previously described (Jarvis, et al., 2014). Transformed AXT3K cells were selected on synthetic dropout medium lacking both uracil and tryptophan. Salt assays were carried out in alkali cation-free medium (AP; (Rodrigueznavarro & Ramos, 1984)) containing 1 mM KCl with the designated concentrations of NaCl and cultured at 30° for 4 days.

To monitor the level of CBL10 expression in yeast, spheroplasts were generated by removing the cell wall with Lyticase (Sigma; (Yilmaz, et al., 2012)). RNA was extracted from spheroplasts using the TRI REAGENT (Invitrogen; (Chomczynksi & Sacchi, 1987; 5006; Yilmaz, et al., 2012)), treated with TURBO DNase (ThermoFisher Scientific) and purified using the RNeasy MiniElute Cleanup kit (Qiagen). cDNA was synthesized using SuperScript III Reverse Transcriptase (Life Technologies) and RNA was removed using RNaseH (Life Technologies). All PCR amplifications were performed using Phusion High-Fidelity DNA Polymerase (ThermoFisher Scientific) and primers are provided in Tables 1 and 2.

Yeast two-hybrid screens. EsCBL10a-interacting kinases were identified using yeast two-hybrid screens. Each gene to be tested for yeast two-hybrid interaction was amplified by PCR using Phusion High-Fidelity DNA Polymerase (ThermoFisher Scientific); primers are provided in Tables 1 and 2. For CIPK genes 1, 2, 4, 6, 7, 9-11, 13, 18, 19, 21, and 24 (SOS2), the PCR products were digested with EcoRI and BamHI and cloned into the corresponding site of the pGADT7 and pGBKT7 vectors (Clonetech/TaKaRa), which allow for expression of the genes as fusion proteins with the GAL4 DNA-activation domain (AD) or the GAL4 DNA-binding domain (BD), respectively. For CIPK genes 3, 5, 8, 12, 14, 15-17, 20, 22, 23, and 26, the PCR products were digested with XmaI and XhoI and cloned into the corresponding site of pGADT7. pGBKT7 does not have a XhoI site but the SalI site produces the same overhang so each CIPK gene digested with XmaI and XhoI was cloned into pGBKT7 digested with XmaI and SalI. For CIPK25, which contains an internal XhoI site, the PCR product was digested with XmaI and SalI and cloned into the corresponding site of pGBKT7 and into pGADT7 digested with XmaI and XhoI. The pGADT7 clones were transformed into Saccharomyces cerevisiae strain Y2HGold (Clontech/TaKaRa) except for the AtSOS3 clone, which had been previously transformed into Saccharomyces cerevisiae strain AH109. The pGBKT7 clones were transformed into Saccharomyces cerevisiae strain Y187 (Clontech/TaKaRa). Yeast were mated and grown on synthetic defined medium (SD) minus leucine and tryptophan (SD-LW, Clonetech/TaKaRa) to select for diploid yeast expressing both constructs. To determine interaction, serial decimal dilutions of yeast colonies were grown on SD-LW and without histidine (H) and adenine (A) or with the addition of 0.5 mM 3-amino-1,2,4-triazole 3-AT (3AT, Sigma) and incubated for five days.

Chimeric proteins. To generate chimeric EsCBL10 for expression in yeast, EsCBL10a and EsCBL10b were cloned into pDR195 (cloning described in yeast salt screens section above). For the first set of chimeric proteins, HindIII was then used to generate two sites in the plasmid, one in the vector and the other within each gene. The resulting fragments containing the N-terminus of each gene were then cloned into the vector containing the C-terminus of the other gene. The plasmids were digested with AgeI and NotI and a fragment containing the chimeric gene was subcloned into the corresponding site of the pFL32T plasmid to be expressed with AtSOS2 in AXT3K. For the second set of chimeric proteins, each gene fragment (aN2, aC2, bN2, and bC2) was PCR amplified using Phusion High-Fidelity DNA Polymerase (ThermoFisher Scientific); primers are provided in Tables 1 and 2. The primers were designed so that the aN2 and bC2 and the bN2 and aC2 fragments would overlap. The fragments were amplified by PCR and the full-length chimeric gene cloned into pGEM-T Easy (Promega). The plasmid was then digested with XhoI and NotI and a fragment containing the chimeric gene was subcloned into the corresponding site of the pDR195 vector. The resulting plasmid was digested with AgeI and NotI and the fragment containing the chimeric gene was subcloned into the corresponding sites of the pFL32T plasmid to be expressed with AtSOS2 in AXT3K. For the third set of chimeric proteins, EsCBL10a and EsCBL10b cloned into pDR195 were transformed into dam-/dcm-competent cells (New England Biolabs) to get a methylation-free plasmid. The resulting plasmids were digested with AgeI and XbaI and the fragments containing the N-terminus of one gene were cloned into the plasmid containing the C-terminus of the other gene. The resulting plasmid was digested with AgeI and Nod and the fragment containing the chimeric gene was subcloned into the corresponding site of the pFL32T plasmid to be expressed with AtSOS2 in AXT3K.

For expression in Arabidopsis, each chimeric gene was amplified from the yeast plasmids and cloned into pGEM-T Easy (Promega). All PCR amplifications were performed using Phusion High-Fidelity DNA Polymerase (ThermoFisher Scientific); primers are provided in Tables 1 and 2. Each chimeric gene was digested with XhoI and BamHI and subcloned into the corresponding site of the plant binary vector pEZT-NL containing the Cauliflower Mosaic Virus 35S promoter. Transformation of Arabidopsis and generation of homozygous lines were performed as described above (complementation assay).

Statistical analysis. To determine significant differences in growth, experiments were organized and analyzed as a randomized complete block design with genotypes and salt concentrations as treatments, and individual experiments as replicates. Treatment effects were assessed using a full-factorial mixed-model ANOVA in JMP, Version 11 (SAS Institute; 1989-2007). In these analyses, treatments were considered fixed effects and replicates random effects. The normality of the distributions of all dependent variables was analyzed by examining a plot of the residuals from a full-factorial ANOVA of untransformed data. A Shapiro-Wilk test (Shapiro & Wilk, 1965) was performed to assess normality, and Bartlett's (Bartlett, 1937) and Levene's (Levene, 1960) tests were performed to evaluate the homogeneity of variance. Based on the pattern of distribution and the results of these tests, a nonparametric approach was used to analyze the data throughout. Data were rank transformed using Microsoft Excel (function: RANK) followed by an ANOVA and Tukey's honestly significant difference (Tukey's HSD) test for multiple comparisons of means (Conover & Iman, 1981). The HSD values from rank-based ANOVA were then applied to the actual means for each measurement (i.e., not the ranks used in ANOVA). Statistical significance was assigned at P≤0.05 throughout, and all tests of significance were two sided.

### Example 1: Eutrema is a Salt-Tolerant Relative of Arabidopsis

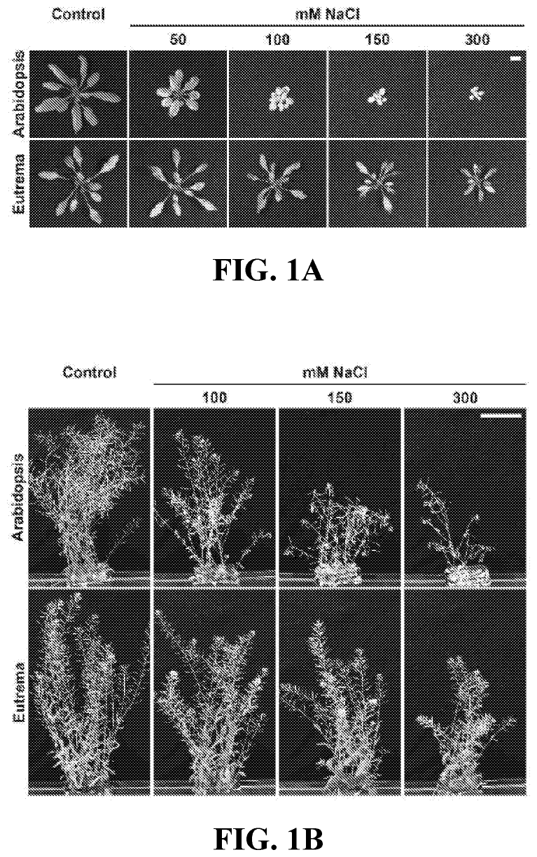

To compare salt tolerance in Arabidopsis and Eutrema, growth of the two species was monitored in the absence and presence of salt during vegetative and reproductive development. Vegetative (rosette) growth in Arabidopsis was reduced at all salt concentrations while Eutrema maintained rosette growth even in the presence of 300 mM NaCl (FIG. 1A). During reproductive development, Arabidopsis inflorescence number, height, and seed production were reduced at all salt concentrations while only small reductions in these traits were found in Eutrema (FIG. 1B). These results, in combination with data from previous studies (Bressan, et al., 2001; In an, et al., 2004), demonstrate that Eutrema is more salt-tolerant than Arabidopsis during both vegetative and reproductive development. Subsequent studies focused on understanding if the duplicated EsCBL10 genes play a role in this salt tolerance.

### Example 2: The Duplicated EsCBL10 Genes Play a Role in Eutrema's Response to Salt

Models describing the retention of duplicated genes predict changes in expression and/or gene function. In this example, EsCBL10a and EsCBL10b transcript accumulation was examined in Eutrema roots, leaves, and flowers and expression of the genes was reduced singly and in combination. In Arabidopsis, CBL10 is alternatively spliced into two primary transcripts; one encoding a functional calcium-binding protein and one encoding a non-functional protein (cannot complement the Atcbl10 mutant) likely due to a premature stop codon in a retained intron (FIG. 3; (Quan et al., 2007)). RT-PCR was used to determine the expression patterns of the Eutrema CBL10 genes and to identify alternatively spliced transcripts. The results show that the expression of EsCBL10b in Eutrema was most similar to expression of AtCBL10 in Arabidopsis; both transcripts were found in aerial tissues (FIG. 2A). When comparing transcript accumulation in leaves and flowers, the EsCBL10b transcript was present at similar levels in both organs whereas the AtCBL10 transcript was found predominantly in leaves and only weakly in flowers (FIG. 2A). EsCBL10a was expressed throughout Eutrema; with similar transcript levels in roots, leaves, and flowers (FIG. 2A). Multiple bands were observed indicating that all three genes are alternatively spliced (FIG. 2A). Gene structures of the EsCBL10 splice variants were determined by cloning and sequencing ten transcripts from each band; multiple variants with different splicing patterns were identified (FIG. 3). If translated, the majority of these variants would likely encode non-functional proteins due to premature stop codons or removal of domains required for binding calcium (FIG. 3). EsCBL10a and EsCBL10b transcripts whose exon/intron structures were most similar to AtCBL10 and likely encode functional proteins (shown in FIG. 2B and listed first in FIG. 3), were used in experiments described below.

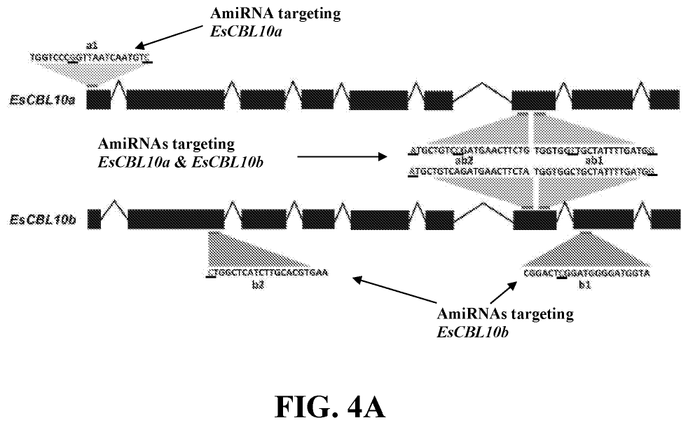

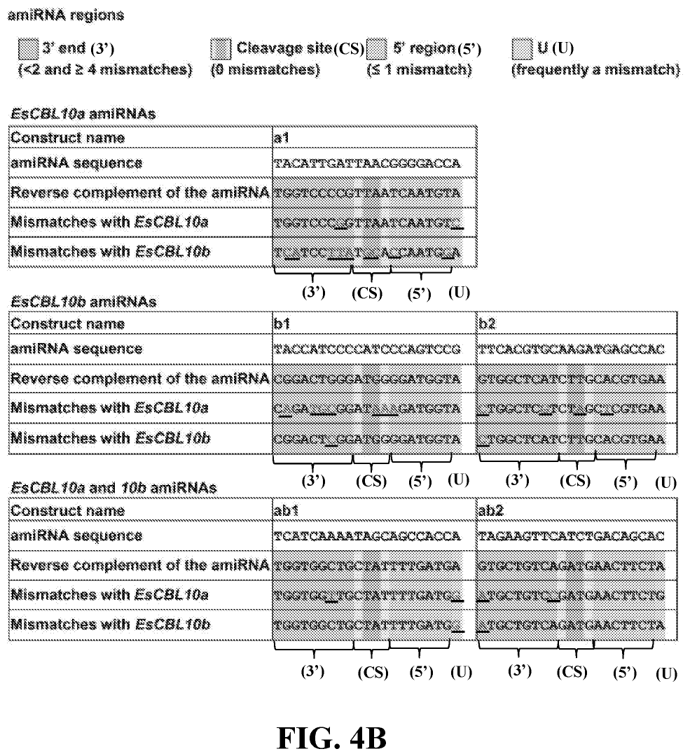

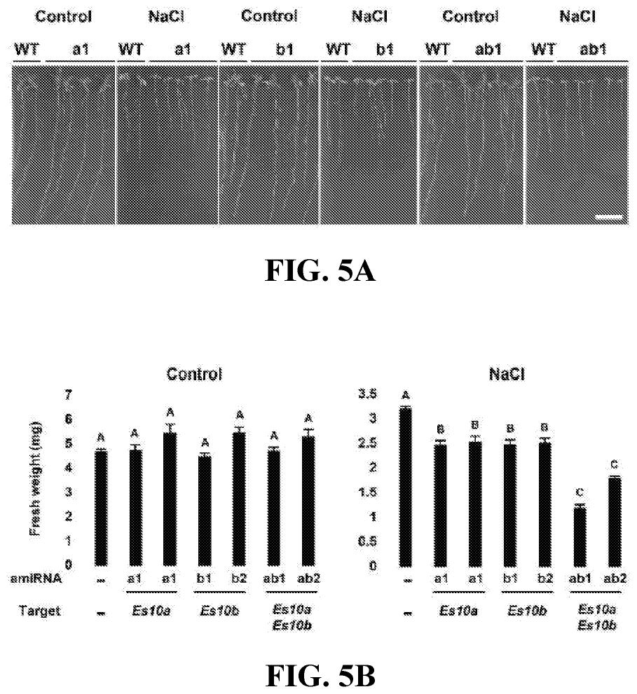

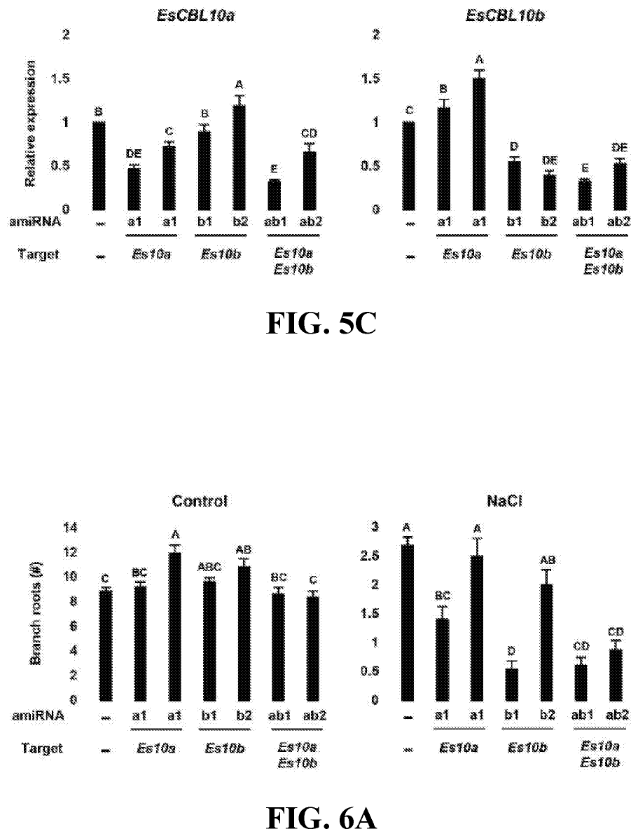

To determine if the EsCBL10 duplication contributes to Eutrema's adaptation to salt, six artificial microRNAs (amiRNAs) were designed to reduce expression of EsCBL10a and EsCBL10b individually and in combination (FIGS. 4A-4B). Two amiRNAs were designed to target EsCBL10a singly but only one was specific; the other reduced transcript levels of both EsCBL10a and EsCBL10b. The salt tolerance of nine independently transformed, single insertion, homozygous lines for each target gene/s was monitored. Two representative lines were chosen; one line per amiRNA construct except for EsCBL10a for which only one amiRNA construct was used. Seedling weight and root length of seedlings grown on solid media were measured as likely components of salt tolerance (shoot biomass or yield) to allow small growth differences associated with down-regulation of one or both genes to be measured. Reduced expression of EsCBL10a and EsCBL10b singly and in combination did not affect growth in control conditions but decreased growth in the presence of salt (FIGS. 5A and 5B). Compared to wild type, in amiRNA lines targeting EsCBL10a, transcript accumulation was reduced (56 and 32%) resulting in seedling hypersensitivity to salt (23 and 21% decrease in fresh weight) (FIGS. 5B and 5C). In amiRNA lines targeting EsCBL10b, transcript accumulation was reduced (45 and 61%) resulting in seedling hypersensitivity to salt (23 and 22% decrease in fresh weight) (FIGS. 5B and 5C). In amiRNA lines targeting both genes, EsCBL10a and EsCBL10b transcript accumulation of EsCBL10a and EsCBL10b was reduced (64 and 36% and 61 and 42%, respectively) resulting in even greater hypersensitivity to salt (63 and 45% decrease) (FIGS. 5B and 5C). These results suggest that both EsCBL10a and EsCBL10b function in Eutrema's response to salt and may have quantitative effects or different roles in salt tolerance.

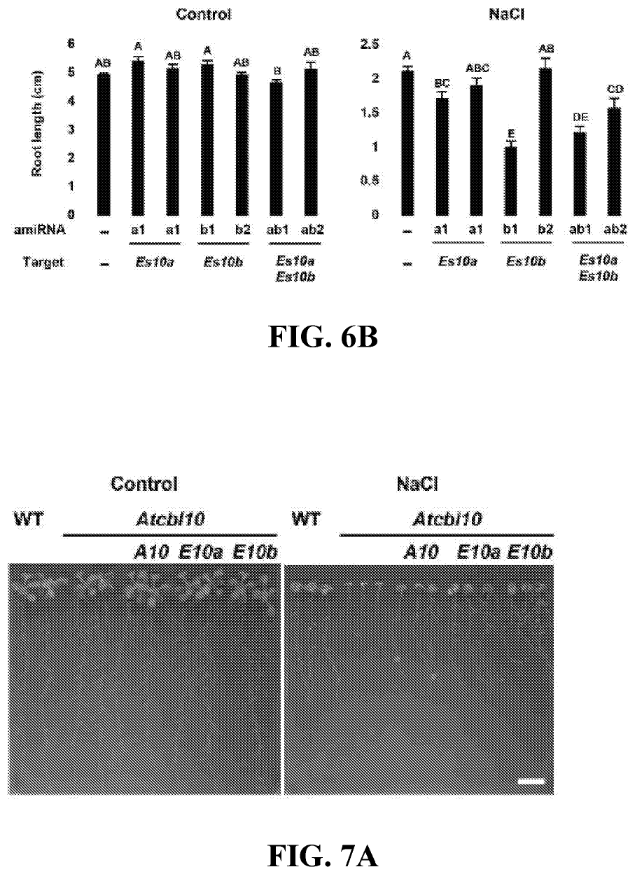

When grown in the presence of salt, lines with reduced expression of both EsCBL10a and EsCBL10b, had fewer branch roots (77 and 67% decrease) and shorter primary roots (43 and 26% decrease) compared to wild type (FIGS. 6A and 6B). The effect of salt on root development in lines targeting EsCBL10a and EsCBL10b individually was variable (FIGS. 6A and 6B). These results suggest that the two genes may have overlapping roles in root development during growth in the presence of salt.

### Example 3: EsCBL10a and EsCBL10b Function in Distinct Pathways in Response to Salt

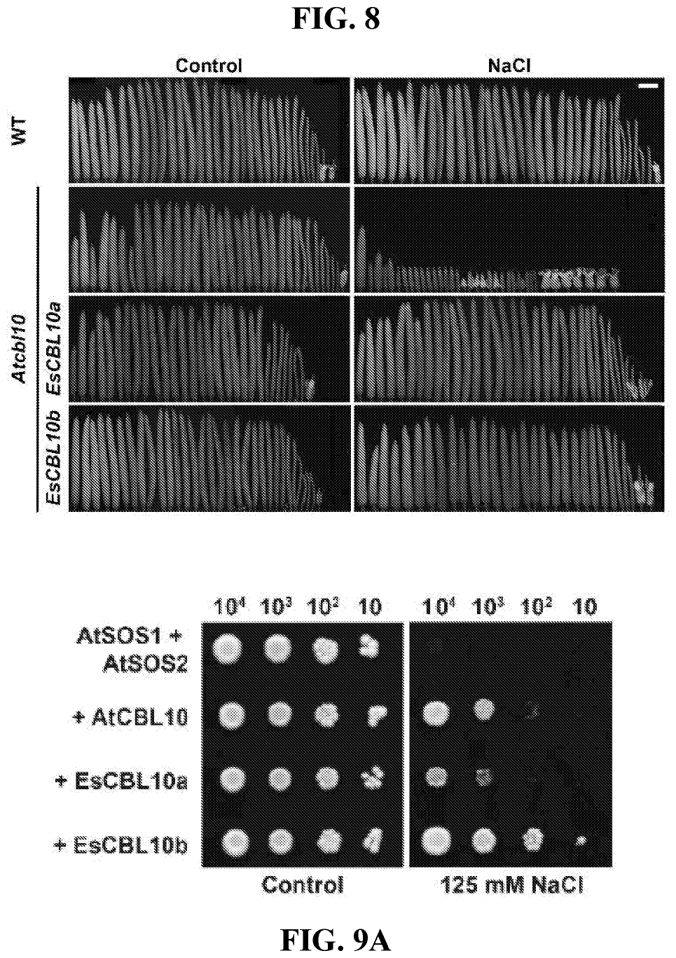

Cross-species complementation assays were used to provide additional insight into the functions of the EsCBL10 genes. Because EsCBL10a and EsCBL10b are orthologous to AtCBL10, their ability to complement the Arabidopsis cbl10 mutant (Atcbl10) was determined. Each gene was expressed in Atcbl10 under control of the constitutive Cauliflower Mosaic Virus 35S (CaMV 35S) promoter and the salt tolerance of five independently transformed, single insertion, homozygous lines was determined for each gene. Both EsCBL10a and EsCBL10b complemented the Atcbl10 salt-sensitive phenotype in all lines examined; however, the degree of complementation varied relative to wild-type Arabidopsis. Strongly complementing lines fully restored growth to wild type-like levels while weakly complementing lines restored growth to 45% of that in wild type—representative strong and weak lines for each are shown (FIGS. 7A and 7B). Transcript accumulation of EsCBL10a and EsCBL10b correlated with the degree of complementation; strongly complementing lines had higher transcript accumulation (FIG. 7C). In Arabidopsis, loss of CBL10 also results in a fertilization defect with shorter siliques due to unfertilized ovules (Monihan, et al., 2016). Both EsCBL10a and EsCBL10b restored fertility in the Atcbl10 mutant (FIG. 8). These results demonstrate that there is conservation of CBL10 function in Arabidopsis and Eutrema.

Potential roles for EsCBL10a and EsCBL10b in the Arabidopsis SOS pathway were investigated using a salt-sensitive strain of yeast (AXT3K) expressing SOS2 and SOS1 from either Arabidopsis or Eutrema. In AXT3K cells expressing either pathway, salt tolerance was greatest in those cells expressing EsCBL10b and weakest in those expressing EsCBL10a relative to yeast expressing AtCBL10 (FIGS. 9A and 9B); differences in activation were not due to differences in gene expression (FIG. 9C). Enhanced activation of the SOS pathway by EsCBL10b could be due to stronger interaction with AtSOS2; yeast two-hybrid assays were performed to determine the strength of this interaction. EsCBL10b and AtCBL10 interacted more strongly with AtSOS2 than EsCBL10a (FIG. 9D) and interaction was orientation-specific (FIG. 9E).

Because EsCBL10a functions in plant responses to salt (down-regulation in Eutrema results in increased salt sensitivity and expression complements the Atcbl10 salt-sensitive phenotype), but only weakly activates the SOS pathway, it was determined whether EsCBL10a can function in alternative pathways. Even though AtCBL10 and AtSOS3 both activate the SOS pathway, they do not reciprocally complement the Atcbl10 or Atsos3 salt-sensitive phenotypes indicating that the two proteins also function in distinct pathways (Quan, 2007).

To determine if EsCBL10a and EsCBL10b can function in an Arabidopsis SOS3 pathway, the genes were expressed in Atsos3 downstream of the CaMV 35S promoter and the salt tolerance of five independently transformed, single insertion, homozygous lines was determined for each gene. EsCBL10a but not EsCBL10b complemented the Atsos3 salt-sensitive phenotype in all lines examined; two representative lines of each are shown (FIGS. 10A and 10B). Expression of EsCBL10a in Atsos3 restored rosette growth (fresh weight) to wild-type levels and partially restored root growth (FIG. 10B). Transcript accumulation of EsCBL10a and EsCBL10b was similar suggesting that the inability of EsCBL10b to complement the mutant was not due to lack of gene expression (FIG. 10C). These results suggest that EsCBL10a and EsCBL10b can function in distinct signaling pathways in response to salt; EsCBL10b with an enhanced ability to activate the SOS pathway and EsCBL10a with an ability to complement Atsos3 suggesting that it has a function not performed by EsCBL10b or AtCBL10. These results also demonstrate that the expression of EsCBL10 gene products in a plant is useful in methods for increasing the salt tolerance of a plant in need thereof.

The ability of EsCBL10a to complement Atsos3 in combination with its weaker interaction with AtSOS2 suggested that EsCBL10a may function with a different kinase. In Arabidopsis, SOS2 belongs to the 26-member CIPK family raising the possibility that EsCBL10a might interact with another CIPK to complement the Atsos3 salt-sensitive phenotype. Yeast two-hybrid assays were performed with all 26 CIPK proteins to identify a kinase that interacts specifically with EsCBL10a but not with EsCBL10b or AtCBL10. Four CIPK kinases (AtCIPK13, AtCIPK16, AtCIPK6 and AtCIPK18) specifically interacted with EsCBL10a (FIG. 10D). In the opposite orientation, interaction was not specific (FIG. 10E). Taken together, these results suggest that interaction with different protein partners might underlie the differences in EsCBL10a and EsCBL10b function.

### Example 4: EsCBL10a and EsSOS3 Function in Distinct Pathways in Response to Salt

The ability of EsCBL10a to complement the Atsos3 salt-sensitive phenotype suggests that two genes in Eutrema (EsCBL10a and the AtSOS3 ortholog EsSOS3) may have similar functions. To compare the activities of the two genes, the salt tolerance of Atsos3 seedlings expressing EsCBL10a or EsSOS3 was examined. EsCBL10a and EsSOS3 were found to complement Atsos3 in a tissue-specific manner; EsCBL10a restored rosette growth (fresh weight) while EsSOS3 restored primary root growth to wild type-like levels (FIGS. 10A and 10B). Due to sequence differences between CBL10 and SOS3, primers that anneal to all genes could not be designed. However, primers that amplify the CBL10 genes and those that amplify the SOS3 genes indicate that transcript accumulation of AtCBL10, EsCBL10a, and EsCBL10b and transcript accumulation of AtSOS3 and EsSOS3 were similar (FIG. 10C). The ability of EsSOS3 and EsCBL10a to activate the SOS pathway was compared by expressing the genes in AXT3K with SOS2 and SOS1 from either Arabidopsis or Eutrema. EsSOS3 activated the Arabidopsis and Eutrema SOS pathways better than EsCBL10a but not as well as EsCBL10b (FIGS. 11A and 11B; FIGS. 23A-23C). To determine the extent of EsSOS3's role in salt tolerance, it was expressed in the Atcbl10 mutant and the salt tolerance of five independently transformed, single insertion, homozygous lines was determined. While EsSOS3 did not restore growth to wild type- or EsCBL10a-like levels, seedlings expressing EsSOS3 had longer roots and greater fresh weight than Atcbl10 seedlings (FIG. 7 and FIG. 12). The difference in the ability of EsCBL10a and EsSOS3 to complement Atsos3 and Atcbl10 and to activate the SOS pathway suggests that, while both play a role in response to salt, these calcium sensors have diverged in function.

### Example 5: The Eutrema Calcium Sensors Confer Salt Tolerance to Arabidopsis

Down-regulation of EsCBL10a and EsCBL10b individually and in combination decreased growth of Eutrema indicating that both genes are necessary for Eutrema's salt tolerance. To determine if the duplicated genes are sufficient to confer salt tolerance, EsCBL10a and EsCBL10b were expressed in combination in Atcbl10, and EsCBL10a and EsSOS3 in combination in Atsos3. For these studies, the mutants were used instead of wild-type Arabidopsis to maintain gene composition consistent with what is found in Eutrema. Plants expressing two Eutrema calcium sensors in combination were generated by crossing homozygous plants expressing each gene individually and the salt tolerance of heterozygous seedlings was analyzed.

For plants expressing EsCBL10a and EsCBL10b in combination, two crosses were generated using strongly complementing single-expressing lines; representative crosses generated from strong lines are shown in FIG. 13. Expression of both genes improved growth relative to wild-type Arabidopsis in the presence of NaCl (growth increased by 10%) (FIGS. 13A and 13B). These results demonstrate the ability of EsCBL10a and EsCBL10b to confer salt tolerance and their co-expression can increase growth in the presence of salt above that of wild-type Arabidopsis. Accordingly, these results show that the EsCBL10a and EsCBL10b are useful in methods for increasing salt tolerance in a plant in need thereof.

In crosses expressing EsCBL10a and EsSOS3 in combination, seedlings had longer primary roots; length increased by 15 and 12% relative to wild type in the presence of NaCl (FIGS. 14A and 14B and FIGS. 15A and 15B). Transcript accumulation of EsCBL11a and EsSOS3 was similar in the single and double expressing lines (FIG. 14C and FIG. 15C). These results demonstrate that EsCBL10a and EsSOS3 are sufficient to confer salt tolerance and increase growth in the presence of salt above that of wild-type Arabidopsis. Accordingly, these results show that EsCBL10a and EsSOS3 are useful in methods for increasing salt tolerance in a plant in need thereof.

### Example 6: The EsCBL10a and EsCBL10b-Specific Functions Reside in the N-Terminus

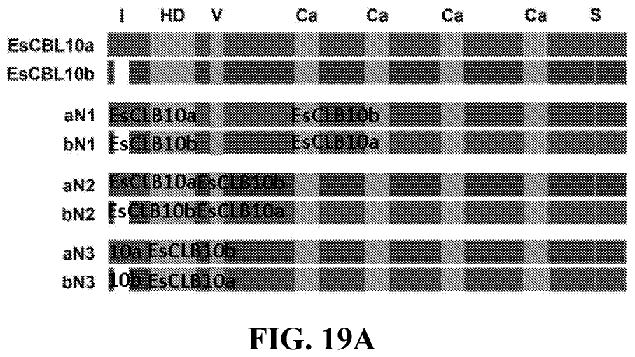

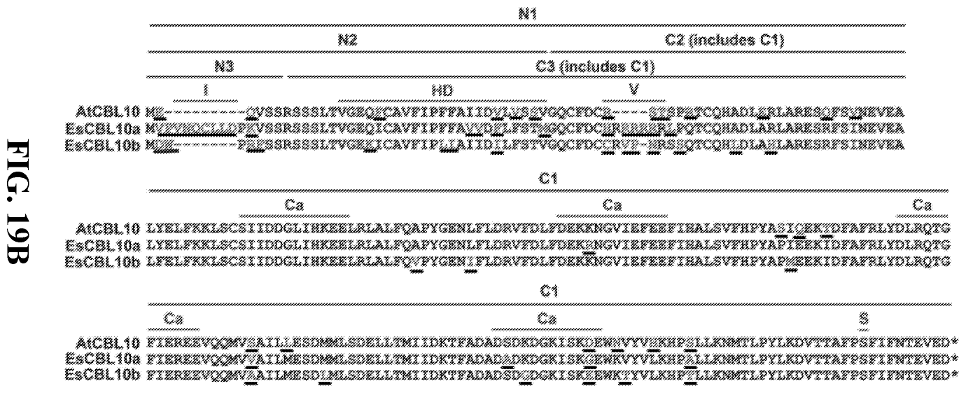

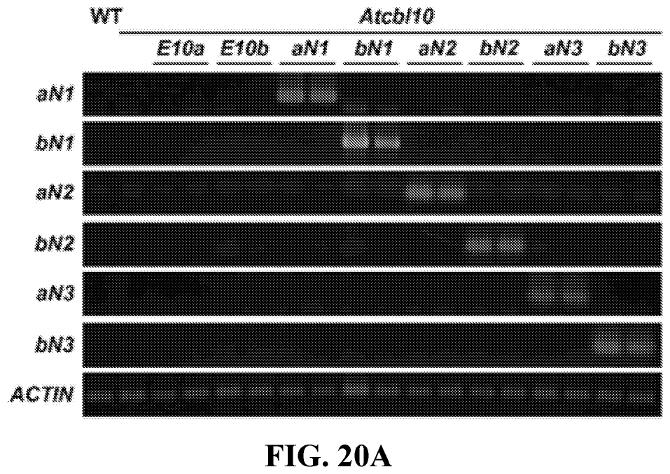

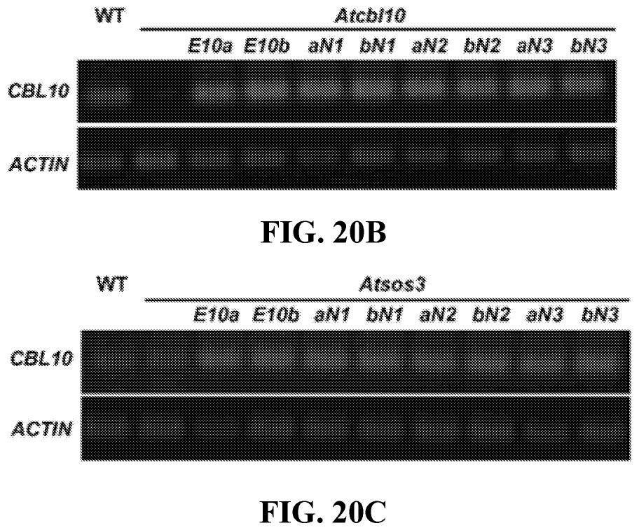

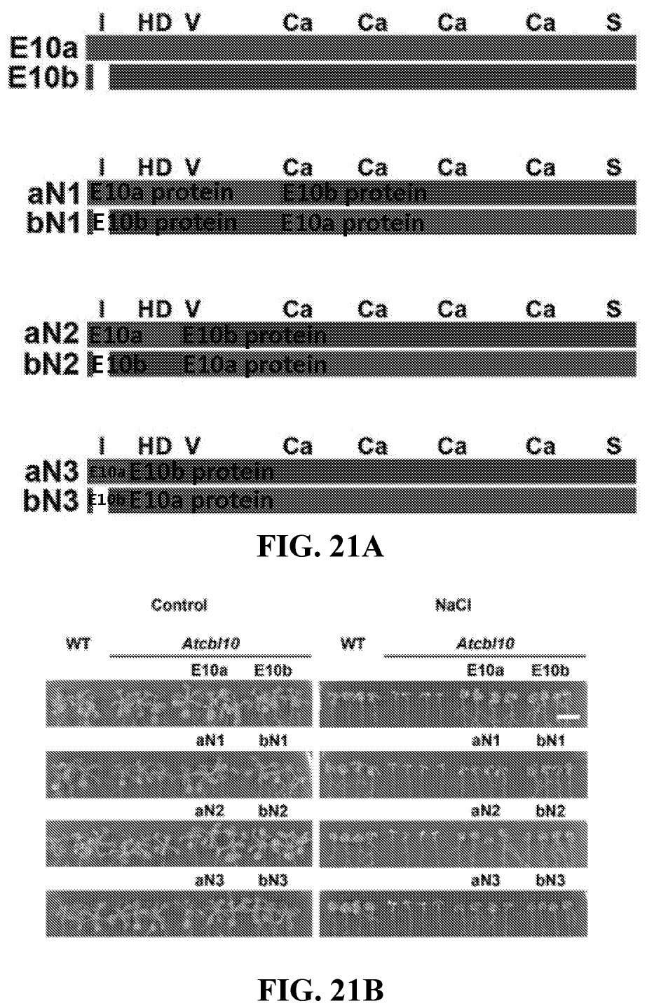

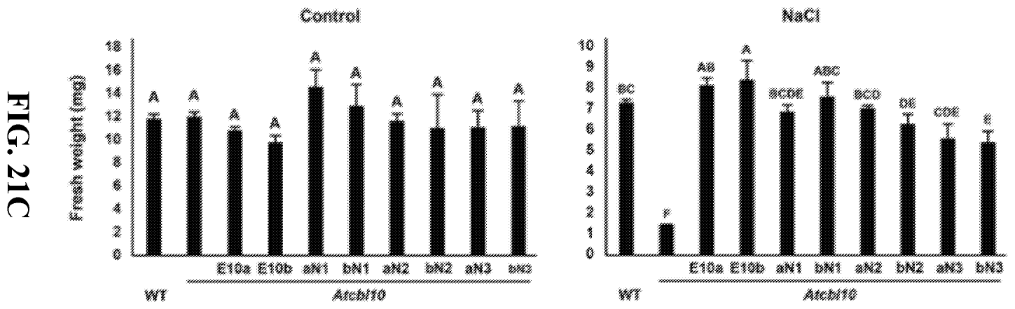

Identification of amino acids or domains that underlie specific functions is critical for understanding how duplication of a gene may contribute to plant adaptation. Because the same promoter was used for all EsCBL10a and EsCBL10b constructs, their distinct functions likely arose through alterations in biochemical functions via changes in their amino acid sequences. In AtCBL10, the hydrophobic domain has been linked to membrane localization, the four EF-hand domains bind calcium, and a serine in the C-terminus of the protein can be phosphorylated to strengthen interaction with AtSOS2 (FIGS. 19A-19B; Quan et al., 2007; Lin et al., 2009). Based on crystal structures, it has been suggested that CBL proteins interact with CIPK proteins through the three-dimensional arrangement of the EF-hand domains, which form a hydrophobic pocket to interact with the CIPK-FISL/NAF domain (a 24 amino acid CIPK-specific domain; (Guo et al., 2001; Sanchez-Barrena et al., 2005; Akaboshi et al., 2008)). Alignment of the EsCBL10 and AtCBL10 proteins identified two additional regions in the N-terminus which may be important for function; an insertion of seven amino acids in EsCBL10a and a variable region in which the amino acid sequence is distinct in all three CBL10 proteins (FIG. 19B). To identify amino acids or domains that underlie the distinct functions of the EsCBL10 proteins, portions of EsCBL10a and EsCBL10b were exchanged to generate three sets of chimeric proteins (FIG. 19A). The first chimeric proteins fused the N-terminal half of one of the EsCBL10 proteins with the C-terminal half of the other (FIG. 19A). The second and third sets of chimeric proteins progressively narrowed down the region underlying the specific functions (FIG. 19A). To verify that the chimeric proteins are functional, each protein was expressed in the Atcbl10 mutant downstream of the CaMV 35S promoter and the salt tolerance of five independently transformed, single insertion, homozygous lines was determined; all chimeric proteins complemented the Atcbl10 salt-sensitive phenotype (FIGS. 20A-20C and FIGS. 21A-21C). To identify regions important for activation of the SOS pathway, the chimeric proteins were expressed in AXT3K along with AtSOS2 and AtSOS1 and growth in the presence of salt was assessed as an indication of pathway activity. Based on these experiments, the ability of EsCBL10b to strongly activate the SOS pathway resides in the first eight amino acids of the protein (FIGS. 18A-18C). To identify regions important for complementation of Atsos3, the chimeric proteins were expressed in the Atsos3 mutant downstream of the CaMV 35S promoter and the salt tolerance of five independently transformed, single insertion, homozygous lines was determined (FIG. 20C). All chimeric proteins with the EsCBL10a hydrophobic domain (aN1, aN2, and bN3) complemented the Atsos3 salt-sensitive phenotype like the full-length protein (major element; FIGS. 22A-22D). Like EsCBL10a, the chimeric protein bN2, which has the EsCBL10b hydrophobic domain and the EsCBL10a variable region, complemented Atsos3 rosette growth (fresh weight) but not root length (minor element; FIGS. 22A-22D). Accordingly, these results demonstrate that the chimeric proteins of the present technology and the EsCBL10a major and minor elements are useful in methods for increasing salt tolerance in a plant in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications, including GenBank Accession Numbers, referred to or cited herein are incorporated by reference in their entirety, including all figures and tables, to the extent they are not inconsistent with the explicit teachings of this specification.

Other embodiments are set forth within the following claims.

### REFERENCES

Amtmann, A. (2009) ‘Learning from evolution: Thellungiella generates new knowledge on essential and critical components of abiotic stress tolerance in plants’, Molecular Plant 2(1): 3-12.
Bartlett, M. S. (1937) ‘Properties of sufficiency and statistical tests’, Proceedings of the Royal Society of London Series a-Mathematical and Physical Sciences 160(A901): 0268-0282.
Bressan, R. A., Zhang, C., Zhang, H., Hasegawa, P. M., Bohnert, H. J. and Zhu, J. K. (2001) ‘Learning from the Arabidopsis experience. The next gene search paradigm’, Plant Physiology 127(4): 1354-60.
Chomczynski, P. and Sacchi, N. (1987) ‘Single-Step Method of Rna Isolation by Acid Guanidinium Thiocyanate Phenol Chloroform Extraction’, Analytical Biochemistry 162(1): 156-159.
Chomczynski, P. and Sacchi, N. (2006) ‘The single-step method of RNA isolation by acid guanidinium thiocyanate-phenol-chloroform extraction: twenty-something years on’, Nature Protocols 1(2): 581-585.
Clough, S. J. and Bent, A. F. (1998) ‘Floral dip: a simplified method for Agrobacterium-mediated transformation of Arabidopsis thaliana’, Plant J 16(6): 735-43.
Conover, W. J. and Iman, R. L. (1981) ‘Rank Transformations as a Bridge between Parametric and Nonparametric Statistics’, American Statistician 35(3): 124-129.
Dassanayake, M., Oh, D. H., Haas, J. S., Hernandez, A., Hong, H., Ali, S., Yun, D. J., Bressan, R. A., Zhu, J. K., Bohnert, H. J. et al. (2011) ‘The genome of the extremophile crucifer Thellungiella parvula’, Nature genetics 43(9): 913-8.
Des Marais, D. L. and Rausher, M. D. (2008) ‘Escape from adaptive conflict after duplication in an anthocyanin pathway gene’, Nature 454(7205): 762-5.
FAO (2002) The salt of the earth: hazardous for food production, www.fao.org/worldfoodsummit/english/newsroom/focus/focus1.htm.
Flagel, L. E. and Wendel, J. F. (2009) ‘Gene duplication and evolutionary novelty in plants’, New Phytol 183(3): 557-64.
Force, A., Lynch, M., Pickett, F. B., Amores, A., Yan, Y. L. and Postlethwait, J. (1999) ‘Preservation of duplicate genes by complementary, degenerative mutations’, Genetics 151(4): 1531-45.
Gonzales-Vigil, E., Bianchetti, C. M., Phillips, G. N., Jr. and Howe, G. A. (2011) ‘Adaptive evolution of threonine deaminase in plant defense against insect herbivores’, Proceedings of the National Academy of Sciences of the United States of America 108(14): 5897-902.
Hahn, M. W. (2009) ‘Distinguishing among evolutionary models for the maintenance of gene duplicates’, J Hered 100(5): 605-17.
Halfter, U., Ishitani, M. and Zhu, J. K. (2000) ‘The Arabidopsis SOS2 protein kinase physically interacts with and is activated by the calcium-binding protein SOS3’, Proc Natl Acad Sci USA 97(7): 3735-40.
Hanikenne, M., Talke, I. N., Haydon, M. J., Lanz, C., Nolte, A., Motte, P., Kroymann, J., Weigel, D. and Kramer, U. (2008) ‘Evolution of metal hyperaccumulation required cis-regulatory changes and triplication of HMA4’, Nature 453(7193): 391-5.
Hoagland, D. R. and Arnon, D. I. (1938) ‘The water-culture method for growing plants without soil’, Calif. Agric. Exp. Station Circular 347: 1-39.
Hughes, A. L. (1994) ‘The evolution of functionally novel proteins after gene duplication’, Proceedings. Biological sciences 256(1346): 119-24.
Ilyas, M., Horger, A. C., Bozkurt, T. O., van den Burg, H. A., Kaschani, F., Kaiser, M., Belhaj, K., Smoker, M., Joosten, M. H., Kamoun, S. et al. (2015) ‘Functional Divergence of Two Secreted Immune Proteases of Tomato’, Current biology: CB 25(17): 2300-6.
Inan, G., Zhang, Q., Li, P., Wang, Z., Cao, Z., Zhang, H., Zhang, C., Quist, T. M., Goodwin, S. M., Zhu, J. et al. (2004) ‘Salt cress. A halophyte and cryophyte Arabidopsis relative model system and its applicability to molecular genetic analyses of growth and development of extremophiles’, Plant Physiology 135(3): 1718-37.
Innan, H. and Kondrashov, F. (2010) ‘The evolution of gene duplications: classifying and distinguishing between models’, Nat Rev Genet 11(2): 97-108.
Jarvis, D. E., Ryu, C. H., Beilstein, M. A. and Schumaker, K. S. (2014) ‘Distinct roles for SOS1 in the convergent evolution of salt tolerance in Eutrema salsugineum and Schrenkiella parvula’, Molecular biology and evolution 31(8): 2094-107.
Kim, B. G., Waadt, R., Cheong, Y. H., Pandey, G. K., Dominguez-Solis, J. R., Schultke, S., Lee, S. C., Kudla, J. and Luan, S. (2007) ‘The calcium sensor CBL10 mediates salt tolerance by regulating ion homeostasis in Arabidopsis’, Plant J 52(3): 473-84.
Kondrashov, F. A. (2012) ‘Gene duplication as a mechanism of genomic adaptation to a changing environment’, Proceedings of the Royal Society B-Biological Sciences 279(1749): 5048-5057.
Levene, H. (1960) Robust tests for equality of variances. in I. Olkin (ed.) Contributions to Probability and Statistics. Stanford: Stanford University Press.
Lin, H., Yang, Y., Quan, R., Mendoza, I., Wu, Y., Du, W., Zhao, S., Schumaker, K. S., Pardo, J. M. and Guo, Y. (2009) ‘Phosphorylation of SOS3-LIKE CALCIUM BINDING PROTEIN8 by SOS2 protein kinase stabilizes their protein complex and regulates salt tolerance in Arabidopsis’, Plant Cell 21(5): 1607-19.
Liu, J., Ishitani, M., Halfter, U., Kim, C. S. and Zhu, J. K. (2000) ‘The Arabidopsis thaliana SOS2 gene encodes a protein kinase that is required for salt tolerance’, Proc Natl Acad Sci USA 97(7): 3730-4.
Liu, J. and Zhu, J. K. (1997) ‘An Arabidopsis mutant that requires increased calcium for potassium nutrition and salt tolerance’, Proc Natl Acad Sci USA 94(26): 14960-4.
Liu, J. and Zhu, J. K. (1998) ‘A calcium sensor homolog required for plant salt tolerance’, Science 280(5371): 1943-5.
Lynch, J. P. (2013) ‘Steep, cheap and deep: an ideotype to optimize water and N acquisition by maize root systems’, Annals of Botany 112(2): 347-57.
Monihan, S. M., Magness, C. A., Yadegari, R., Smith, S. E. and Schumaker, K. S. (2016) ‘Arabidopsis CALCINEURIN B-LIKE10 Functions Independently of the SOS Pathway during Reproductive Development in Saline Conditions’, Plant Physiology 171(1): 369-79.
Munns, R. and Tester, M. (2008) ‘Mechanisms of salinity tolerance’, Annu Rev Plant Biol 59: 651-81.
Oh, D. H., Leidi, E., Zhang, Q., Hwang, S. M., Li, Y., Quintero, F. J., Jiang, X., D'Urzo, M. P., Lee, S. Y., Zhao, Y. et al. (2009) ‘Loss of halophytism by interference with SOS1 expression’, Plant Physiol 151(1): 210-22.
Ohno, S. (1970) Evolution by gene duplication. Berlin, Germany: Springer.
Ossowski, S., Schwab, R. and Weigel, D. (2008) ‘Gene silencing in plants using artificial microRNAs and other small RNAs’, The Plant journal: for cell and molecular biology 53(4): 674-90.
Qadir, M., Quillerou, E., Nangia, V., Murtaza, G., Singh, M., Thomas, R. J., Drechsel, P. and Noble, A. D. (2014) ‘Economics of salt-induced land degradation and restoration’, Natural Resources Forum 38(4): 282-295.
Qiu, Q. S., Barkla, B. J., Vera-Estrella, R., Zhu, J. K. and Schumaker, K. S. (2003) ‘Na+/H+ exchange activity in the plasma membrane of Arabidopsis’, Plant Physiol 132(2): 1041-52.
Qiu, Q. S., Guo, Y., Dietrich, M. A., Schumaker, K. S. and Zhu, J. K. (2002) ‘Regulation of SOS1, a plasma membrane Na⁺/H⁺ exchanger in Arabidopsis thaliana, by SOS2 and SOS3’, Proc Natl Acad Sci USA 99(12): 8436-41.
Quan, R., Lin, H., Mendoza, I., Zhang, Y., Cao, W., Yang, Y., Shang, M., Chen, S., Pardo, J. M., Guo, Y. (2007) ‘SCABP8/CBL10, a putative calcium sensor, interacts with the protein kinase SOS2 to protect Arabidopsis shoots from salt stress’, Plant Cell 19(4): 1415-31.
Quintero, F. J., Ohta, M., Shi, H., Zhu, J. K. and Pardo, J. M. (2002) ‘Reconstitution in yeast of the Arabidopsis SOS signaling pathway for Na+ homeostasis’, Proc Natl Acad Sci USA 99(13): 9061-6.
Rentsch, D., Laloi, M., Rouhara, I., Schmelzer, E., Delrot, S. and Frommer, W. B. (1995) ‘Ntrl Encodes a High-Affinity Oligopeptide Transporter in Arabidopsis’, Febs Letters 370(3): 264-268.
Rodrigueznavarro, A. and Ramos, J. (1984) ‘Dual System for Potassium-Transport in Saccharomyces-Cerevisiae’, Journal of Bacteriology 159(3): 940-945.
Schmittgen, T. D. and Livak, K. J. (2008) ‘Analyzing real-time PCR data by the comparative C(T) method’, Nature protocols 3(6): 1101-8.
Schwab, R., Ossowski, S., Riester, M., Warthmann, N. and Weigel, D. (2006) ‘Highly specific gene silencing by artificial microRNAs in Arabidopsis’, The Plant cell 18(5): 1121-33.
Shapiro, S. S. and Wilk, M. B. (1965) ‘An Analysis of Variance Test for Normality (Complete Samples)’, Biometrika 52: 591-&.
Shi, H., Ishitani, M., Kim, C. and Zhu, J. K. (2000) ‘The Arabidopsis thaliana salt tolerance gene SOS1 encodes a putative Na⁺/H⁺ antiporter’, Proc Natl Acad Sci USA 97(12): 6896-901.
Simon, S., Ruhl, M., de Montaigu, A., Wotzel, S. and Coupland, G. (2015) ‘Evolution of CONSTANS Regulation and Function after Gene Duplication Produced a Photoperiodic Flowering Switch in the Brassicaceae’, Molecular biology and evolution 32(9): 2284-301.
Smyth, D. R., Bowman, J. L. and Meyerowitz, E. M. (1990) ‘Early flower development in Arabidopsis’, Plant Cell 2(8): 755-67.
Tang, R. J., Yang, Y., Yang, L., Liu, H., Wang, C. T., Yu, M. M., Gao, X. S. and Zhang, H. X. (2014) ‘Poplar calcineurin B-like proteins PtCBL10A and PtCBL10B regulate shoot salt tolerance through interaction with PtSOS2 in the vacuolar membrane’, Plant Cell Environ 37(3): 573-88.
Wu, S. J., Ding, L. and Zhu, J. K. (1996) ‘SOS1, a Genetic Locus Essential for Salt Tolerance and Potassium Acquisition’, Plant Cell 8(4): 617-627.
Yang, R., Jarvis, D. E., Chen, H., Beilstein, M. A., Grimwood, J., Jenkins, J., Shu, S., Prochnik, S., Xin, M., Ma, C. et al. (2013) ‘The Reference Genome of the Halophytic Plant Eutrema salsugineum’, Frontiers in plant science 4: 46.
Yilmaz, R., Akça, O., Baloğlu, M. C., Öz, M. T., Öktem, H. A. and Yücel, M. (2012) ‘Optimization of yeast (Saccharomyces cerevisiae) RNA isolation method for real-time quantitative PCR and microarray analysis’, African Journal of Biotechnology 11(5): 1046-1053.
Zhu, J. K., Liu, J. and Xiong, L. (1998) ‘Genetic analysis of salt tolerance in Arabidopsis. Evidence for a critical role of potassium nutrition’, Plant Cell 10(7): 1181-91.

SEQUENCE LISTING
EsCBL10a gene
SEQ ID NO: 1 (1935 bp)
AAATACAATACCTCTCATCCAGAATTTCGAAATCTTGTGATGTGATCTCGACACCATCTTGG
AAGTCGGCGAGGACGAAGAAGAGGACTTAGAGGAGGATGGTCCCGGTTAATCAATGTCTCCT
GGACCCGAAAGTTTCCTCTGTAAATTTCTCTCATTTCGCTTTGATTTAGTTTTCTCCTTTCT
TCTCTCTCTTGGTTTTGGATTGAAGGTAATGTTTTGGGTCTTCCTTTGTTAAAACAGAGATC
AAGCTCTCTTACAGTTGGAGAGCAAATCTGCGCCGTCTTCATACCTTTCTTTGCCGTTGTTG
ATTTCCTCTTCTCCACCATGGGACAATGCTTCGATTGCCACCGCCGCCGCCGCCGGCGGTTG
CCTCAGACATGTCAGCACGCGGATCTGGCTCGTCTAGCTCGTGAATCTCGATGTGGGTTTTC
TTCTTTTCACCCTCTCTCTCTCTGGACCAATTCTTTGTCAATGGGTTTTACAATTTCTCTGT
TATGGAGCTCAAAAGTTTACAACTTTGGTTCGAATGTACTTGTAGTTTCAATCAACGAAGTT
GAAGCTTTGTATGAGCTATTCAAGAAACTGAGCTGTTCCATTATTGATGACGGCTTGATTCA
CAAGGTAAGTTTATTTTTTCTTGTCTCGGATACTAGTGAATAGAGATCCGGTTGTTGAATTT
GCCATAGGTTGAAGCTTCTATGTGTAAATTCTTTTCTTGTGCTGATGAGTATTGGTTGTGAG
AATATCGATGTTCCTGGTTTGTAGGAAGAGCTTAGACTGGCCCTGTTCCAAGCTCCATATGG
CGAGAACCTCTTTCTTGATAGGGTGAGTAGATGTGACTATGTCCTTTGGAGAAGACTGATTA
GCTTTTCGTGGTTTCTTGGACTTGTTGAGATGAAAGTTAATGATTATATACTCGAAGTTTTG
TAGGTTTTTGATTTGTTCGATGAAAAGAGGAATGGTGTTATTGAGTTTGAGGAATTCATCCA
TGCATTGAGTGTCTTTCATCCCTATGCACCGATTGAGGAGAAGATCGACTGTAAGTTTCATT
GAAACAATTATCTTAAGAAAAGGGATCATTGAACCAAAATTTTGTGTCAGTTGAACAATTAT
TAGTAATATATATAACCCAGGGATGAGTCTCAAGCATATGTTTTTAATCTGTGGCAGTTGCA
TTTAGGCTCTATGATCTAAGACAAACCGGATTTATTGAGCGAGAAGAGGTAAATGATTGTGC
CCACTGCAAGTTGCAACTGTATTTTGAGCTCAATAACAAGTTTTAAGCTGCTAATTTTAGGT
GCAACAAATGGTGGTTGCTATTTTGATGGAATCTGATATGATGCTGTCCGATGAACTTCTGA
CAATGATTATTGATAAAGTGAGTGTAATTGGAATCCTTTCTCGTAGTGATGACTCATATTTG
TTAAATTCAGTTAGCAGATGGGGTTTTTTTTAATCCTCTTCATTGTCTTTTGCTAGACATTT
GCTGATGCAGATGCGGATAAAGATGGTAAAATTAGCAAGGGAGAATGGAAGGTGTATGTGCT
TAAGCATCCCGCCTTATTGAAGAATATGACTCTTCCTTACCTAAAGTACCAATTTCTTTTCT
CTCTCTATTTGTTAGATTAACTCTCTAAATCTAAGATAACCTCTACTTTGTGATCATTAACT
TATCTGTTTGAATTTGAAGAGATGTGACGACAGCATTTCCAAGTTTTATATTCAACACAGAG
GTTGAAGACTGAGGCAGAACCGTGTGTGACCTTCTTCTGCTTGGTTAACGTACTAAAGCCGT
TATTTATTTTAAATGTTTTTAAACTCAAATTAGTTGGTGATTATTAGATGTAAGTAAAAAAT
GTGAGGTTTTGCCATCATTTGATGCACCCATTTGTAGACTGTTACATTGAAAATGTAATGAA
GCATTCTTTTTTC
EsCBL10a ORF
SEQ ID NO: 2 (780 bp)
ATGGTCCCGGTTAATCAATGTCTCCTGGACCCGAAAGTTTCCTCTAGATCAAGCTCTCTTAC
AGTTGGAGAGCAAATCTGCGCCGTCTTCATACCTTTCTTTGCCGTTGTTGATTTCCTCTTCT
CCACCATGGGACAATGCTTCGATTGCCACCGCCGCCGCCGCCGGCGGTTGCCTCAGACATGT
CAGCACGCGGATCTGGCTCGTCTAGCTCGTGAATCTCGATTTTCAATCAACGAAGTTGAAGC
TTTGTATGAGCTATTCAAGAAACTGAGCTGTTCCATTATTGATGACGGCTTGATTCACAAGG
AAGAGCTTAGACTGGCCCTGTTCCAAGCTCCATATGGCGAGAACCTCTTTCTTGATAGGGTT
TTTGATTTGTTCGATGAAAAGAGGAATGGTGTTATTGAGTTTGAGGAATTCATCCATGCATT
GAGTGTCTTTCATCCCTATGCACCGATTGAGGAGAAGATCGACTTTGCATTTAGGCTCTATG
ATCTAAGACAAACCGGATTTATTGAGCGAGAAGAGGTGCAACAAATGGTGGTTGCTATTTTG
ATGGAATCTGATATGATGCTGTCCGATGAACTTCTGACAATGATTATTGATAAAACATTTGC
TGATGCAGATGCGGATAAAGATGGTAAAATTAGCAAGGGAGAATGGAAGGTGTATGTGCTTA
AGCATCCCGCCTTATTGAAGAATATGACTCTTCCTTACCTAAAAGATGTGACGACAGCATTT
CCAAGTTTTATATTCAACACAGAGGTTGAAGACTGA
EsCBL10a polypeptide
SEQ ID NO: 3 (259 AA)
MVPVNQCLLDPKVSSRSSSLTVGEQICAVFIPFFAVVDFLFSTMGQCFDCHRRRRRRLPQTC
QHADLARLARESRFSINEVEALYELFKKLSCSIIDDGLIHKEELRLALFQAPYGENLFLDRV
FDLFDEKRNGVIEFEEFIHALSVFHPYAPIEEKIDFAFRLYDLRQTGFIEREEVQQMVVAIL
MESDMMLSDELLTMIIDKTFADADADKDGKISKGEWKVYVLKHPALLKNMTLPYLKDVTTAF
PSFIFNTEVED*
EsCBL10b gene
SEQ ID NO: 4 (2003 bp)
CAAGATTTGGAAAAATTATGATGTGATTCAAAACCAAATCATTTAGACGAAGAAGAAGAAGA
GGAGTTGTGCTTCAATGAGTCATGGACTGGCCCAGATTTTCCTCTGTAATTTTCCTATTTCT
AATTTCTTCTACTCTATTTTTGGGTTTTGTGTAATGTTTTGGATCTTCCTTTCTCTAAACAG
AGATCCAGTTCTTTGACTGTTGGGGAGAAAATCTGCGCCGTCTTCATACCTTTAATCGCCAT
TATTGATATTCTCTTCTCCACCGTTGGACAGTGCTTTGATTGCTGCCGTGTCCCCAATCGAT
CGTCTCAGACATGTCAGCACCTGGATCTGGCTCATCTTGCACGTGAATCTCGATGTGGGTTT
TCTTGTTTTCACTCTCTTATCAATGGTTTTGGAAATTATCTTGTTTTGTAGTTCAAAAGTTT
TCTAAGCTTTGGTTTGTTTTGTTTGCTTTTACGCAGTTTCGATCAATGAAGTTGAAGCTTTG
TTTGAGCTCTTCAAGAAATTGAGCTGTTCAATTATTGATGACGGCTTGATTCACAAGGTAAG
TTTTTTTTTTTGTTTTTCTCTCATTCTTTTTGGAAAGTATCTTAGAATATTCATCTGTTTGA
CATTTTAATGTGTGATTGCTTTCAAATCAAAATGAATGGTTTGCTCTTTTAGCTGATCATTA
CTCATCTCATAAAAGTGTCATTGTTTTTTTTCCCTCTTATGAAATGATTAATTATGAGAATT
TTGATATATTCTTGTTTTGTAGGAAGAGCTTCGACTGGCCCTGTTCCAAGTTCCATATGGTG
AGAACATCTTTCTTGATAGGGTGAGTGTGTTTCTTCAGAGAGACGAAATAGCTTACTTCATG
GTTTCTTGGACTTGTTGAGATGAAAGTTATGATTATATATATATATAAATTGATGTTATGTA
GGTTTTCGATTTGTTTGATGAAAAGAAGAATGGGGTTATTGAGTTTGAGGAATTTATCCATG
CATTAAGTGTCTTTCATCCTTATGCACCAATGGAGGAGAAGATCGACTGTAAGGATCATGGA
AAAAAAAACTCAATGTCCTATATATAGCAAAGCTTTGTCATATAACCCATGGATATGAGTCT
CAAACATATGTTTCTAATCTGTTGGCAGTTGCATTTAGGCTCTATGATTTGAGACAAACCGG
ATTTATCGAGCGCGAAGAGGTAAACATTATGTGCCTTGCCCATTTTGATTTCTTAGTTGTCT
TTTTAAGCTAAATAAAAAGTTGAAGTTGTTAAATGTTAAGGTACAACAAATGGTGGCTGCTA
TTTTGATGGAATCTGATTTGATGCTGTCAGATGAACTTCTAACCATGATTATCGATAAAGTG
AGTGTATTTGAAATAATTTATCTTAGTGATGAATCATGTTTTGGTATGTTTATCTTTTCTCT
ACTTTTTTTTTGTTTGGCTAGACATTTGCTGATGCGGACTCGGATGGGGATGGTAAAATTAG
CAAGGAGGAGTGGAAGACTTATGTGCTTAAGCATCCAACCTTGTTAAAGAATATGACTCTTC
CTTACTTAAAGTACCAAATCTACTCTCTTTGTTAGATTAACTCAGTCATTAAAAACAAAGCC
TTTACGTTATGACCAACACTTATCTACTTGAATTTGAAGGGATGTGACAACAGCATTCCCAA
GTTTTATATTCAATACTGAGGTTGAAGACTGATGCAGAAGCATGTCCATAAAACAACCGGTA
ATTTTTTTTTCTGCTTGGCTTACATATTTTTCTATATGTTGTATATATATGTAAACCAAGCA
GAAAATAAATTTTGATTTACATATAAGAACGGATATCTTTATTGTATTTTACCCAAATTGGT
TGGTGATTATTAAATGTGAGAAATGTTGTGAGGTTCCATCGAAGGATTTACCTATCCTTCTC
TTGCGCGCATTGTGGACTAATACATTGGAAGTAACTAAGCTTTTTTGTTCCAAGGGGAAAGA
GTGGAAAAGGGAAAAAAAA
EsCBL10b ORF
SEQ ID NO: 5 (756 bp)
ATGGACTGGCCCAGATTTTCCTCTAGATCCAGTTCTTTGACTGTTGGGGAGAAAATCTGCGC
CGTCTTCATACCTTTAATCGCCATTATTGATATTCTCTTCTCCACCGTTGGACAGTGCTTTG
ATTGCTGCCGTGTCCCCAATCGATCGTCTCAGACATGTCAGCACCTGGATCTGGCTCATCTT
GCACGTGAATCTCGATTTTCGATCAATGAAGTTGAAGCTTTGTTTGAGCTCTTCAAGAAATT
GAGCTGTTCAATTATTGATGACGGCTTGATTCACAAGGAAGAGCTTCGACTGGCCCTGTTCC
AAGTTCCATATGGTGAGAACATCTTTCTTGATAGGGTTTTCGATTTGTTTGATGAAAAGAAG
AATGGGGTTATTGAGTTTGAGGAATTTATCCATGCATTAAGTGTCTTTCATCCTTATGCACC
AATGGAGGAGAAGATCGACTTTGCATTTAGGCTCTATGATTTGAGACAAACCGGATTTATCG
AGCGCGAAGAGGTACAACAAATGGTGGCTGCTATTTTGATGGAATCTGATTTGATGCTGTCA
GATGAACTTCTAACCATGATTATCGATAAAACATTTGCTGATGCGGACTCGGATGGGGATGG
TAAAATTAGCAAGGAGGAGTGGAAGACTTATGTGCTTAAGCATCCAACCTTGTTAAAGAATA
TGACTCTTCCTTACTTAAAGGATGTGACAACAGCATTCCCAAGTTTTATATTCAATACTGAG
GTTGAAGACTGA
EsCBL10b polypeptide
SEQ ID NO: 6 (251 AA)
MDWPRFSSRSSSLTVGEKICAVFIPLIAIIDILFSTVGQCFDCCRVPNRSSQTCQHLDLAHL
ARESRFSINEVEALFELFKKLSCSIIDDGLIHKEELRLALFQVPYGENIFLDRVFDLFDEKK
NGVIEFEEFIHALSVFHPYAPMEEKIDFAFRLYDLRQTGFIEREEVQQMVAAILMESDLMLS
DELLTMIIDKTFADADSDGDGKISKEEWKTYVLKHPTLLKNMTLPYLKDVTTAFPSFIFNTE
VED*
EsSOS3 gene
SEQ ID NO: 7 (1517 bp)
ATGGGCTGCTCTCCGTCGAAGAGGAAGAAGGCAACACGACCGCCGGGATATGAGGATCCTAA
CCTTCTCGCCTCCGCTACGCCATGTCAGTTTTAAGTTCTTGATCTTGTTAATATGATACTCG
TTAGAAAATACACATAATAGACTTCGGACAAAACTCCAATCTAATTCTACTTTTGATATTGG
GAGGGACAATAGTCACGGTAGCAGAAGTGGAGGCTTTGTATGAACTGTTCATGAAGCTAAGC
AGTTCAATTATCGACGACGGTCTTATTCATAAGGTTTCATTCTTTCTTTTTCATCCAATATA
CTCTTTAACATTATCGAAAACAGAGTCCTCTGCTTTGCTTTTTGAGATCTTTTGTATTCATA
TATATATATATATATATATATATATATATTGGCTTAGAGTTTAAAGTGTGATTGATGTTGTA
GGAAGAATTTCAGCTGGCTTTATTCAGAAATAGGAATCGGAAGAACCTTTTCGCTGATCGGG
TACGTATATATCAACTACCTACAGGCTACAAAAAAGTTACACATTTTTAACAAAAGAAACAT
ATGTATTTTGTAAATGACGTTTTGTTTTATGATATTTTATCCGCAGATATTTGATGTATTTG
ATGTGAAGCGAAATGGAGTGATCGAGTTTGGGGAATTTGTCCGGTCTTTAGGTGTCTTCCAT
CCAGATGCATCTGTCCATGAAAAAATCAAATGTAACTTTTTCTAAACCCCTCCACATTGCTT
CACATTCATTCTATGTGTTAAAGTGTTCTTTCTATGTTAACTAACAATGAGTTTTATCCATG
AATTTTTGCAGTTGCTTTCAAGTTGTACGATTTACGGCAAACTGGATTCATCGAGCGGGAAG
AAGTGAGTCCATTTTACGGTTGTTTTCTTGGAACCTGCAAGATTATAGTTTATGTATCACCA
TAACTTTGTGTGTTGTTCTTACGTATAGCTGAAAGAGATGGTAATCGCTCTTCTTCACGAAT
CCGAACTTATACTTTCCGAAGATATGATTGAAGTAATGGTGGATAAGGTGCTCCATCTTTTC
ACACCATTACCTATTTGTTTATTTTTCTATCTATCACTATAGTATATTAACTCGATGTTGAA
GATTCTAATCTTAATATAAGGAGATGGTGTTGTATTATATGCACAGGCTTTTATCGAAGCAG
ATCGCAAGAACGATGGGAGAATTGATATAGATGAATGGAAAGATTTCGTGTCCAAGAATCCG
TCGCTCATCAAAAACATGACTTTGCCGTATCTAAAGTGAGTTTTACATTGACTACTTCAAAG
ATTTATAAAATGTTTGCTCAAAGGAGAACCCATTCTTGAGCCAAACCGACCGGTTTGATTGA
TGATAAGTTTCTTTGTAAAATGTAGGGACATAAAGGGGGCGTTTCCAAGTTTTGTTTCATCT
TGTGAAGACGATGAATTGGAGTTGCAAAAACTAAATTTTTAAGTAACCCAATACAGATTGCA
GATAAAGAAGATAAAAAAAAAGCCCCAAC
EsSOS3 ORF
SEQ ID NO: 8 (663 bp)
ATGGGCTGCTCTCCGTCGAAGAGGAAGAAGGCAACACGACCGCCGGGATATGAGGATCCTAA
CCTTCTCGCCTCCGCTACGCCATTCACGGTAGCAGAAGTGGAGGCTTTGTATGAACTGTTCA
TGAAGCTAAGCAGTTCAATTATCGACGACGGTCTTATTCATAAGGAAGAATTTCAGCTGGCT
TTATTCAGAAATAGGAATCGGAAGAACCTTTTCGCTGATCGGATATTTGATGTATTTGATGT
GAAGCGAAATGGAGTGATCGAGTTTGGGGAATTTGTCCGGTCTTTAGGTGTCTTCCATCCAG
ATGCATCTGTCCATGAAAAAATCAAATTTGCTTTCAAGTTGTACGATTTACGGCAAACTGGA
TTCATCGAGCGGGAAGAACTGAAAGAGATGGTAATCGCTCTTCTTCACGAATCCGAACTTAT
ACTTTCCGAAGATATGATTGAAGTAATGGTGGATAAGGCTTTTATCGAAGCAGATCGCAAGA
ACGATGGGAGAATTGATATAGATGAATGGAAAGATTTCGTGTCCAAGAATCCGTCGCTCATC
AAAAACATGACTTTGCCGTATCTAAAGGACATAAAGGGGGCGTTTCCAAGTTTTGTTTCATC
TTGTGAAGACGATGAATTGGAGTTGCAAAAACTAAATTTTTAA
EsSOS3 polypeptide
SEQ ID NO: 9 (220 AA)
MGCSPSKRKKATRPPGYEDPNLLASATPFTVAEVEALYELFMKLSSSIIDDGLIHKEEFQLA
LFRNRNRKNLFADRIFDVFDVKRNGVIEFGEFVRSLGVFHPDASVHEKIKFAFKLYDLRQTG
FIEREELKEMVIALLHESELILSEDMIEVMVDKAFIEADRKNDGRIDIDEWKDFVSKNPSLI
KNMTLPYLKDIKGAFPSFVSSCEDDELELQKLNF*
aN1 chimeric protein nucleotide sequence
SEQ ID NO: 10
ATGGTCCCGGTTAATCAATGTCTCCTGGACCCGAAAGTTTCCTCTAGATCAAGCT
CTCTTACAGTTGGAGAGCAAATCTGCGCCGTCTTCATACCTTTCTTTGCCGTTGTT
GATTTCCTCTTCTCCACCATGGGACAATGCTTCGATTGCCACCGCCGCCGCCGCC
GGCGGTTGCCTCAGACATGTCAGCACGCGGATCTGGCTCGTCTAGCTCGTGAATC
TCGATTTTCAATCAACGAAGTTGAAGCTTTGTTTGAGCTCTTCAAGAAATTGAGC
TGTTCAATTATTGATGACGGCTTGATTCACAAGGAAGAGCTTCGACTGGCCCTGT
TCCAAGTTCCATATGGTGAGAACATCTTTCTTGATAGGGTTTTCGATTTGTTTGAT
GAAAAGAAGAATGGGGTTATTGAGTTTGAGGAATTTATCCATGCATTAAGTGTCT
TTCATCCTTATGCACCAATGGAGGAGAAGATCGACTTTGCATTTAGGCTCTATGA
TTTGAGACAAACCGGATTTATCGAGCGCGAAGAGGTACAACAAATGGTGGCTGC
TATTTTGATGGAATCTGATTTGATGCTGTCAGATGAACTTCTAACCATGATTATCG
ATAAAACATTTGCTGATGCGGACTCGGATGGGGATGGTAAAATTAGCAAGGAGG
AGTGGAAGACTTATGTGCTTAAGCATCCAACCTTGTTAAAGAATATGACTCTTCC
TTACTTAAAGGATGTGACAACAGCATTCCCAAGTTTTATATTCAATACTGAGGTT
GAAGACTGA
aN1 chimeric protein amino acid sequence
SEQ ID NO: 11
MVPVNQCLLDPKVSSRSSSLTVGEQICAVFIPFFAVVDFLFSTMGQCFDCHRRRR
RRLPQTCQHADLARLARESRFSINEVEALFELFKKLSCSIIDDGLIHKEELRLALF
QVPYGENIFLDRVFDLFDEKKNGVIEFEEFIHALSVFHPYAPMEEKIDFAFRLYD
LRQTGFIEREEVQQMVAAILMESDLMLSDELLTMIIDKTFADADSDGDGKISKE
EWKTYVLKHPTLLKNMTLPYLKDVTTAFPSFIFNTEVED
aN2 chimeric protein nucleotide sequence
SEQ ID NO: 12
ATGGTCCCGGTTAATCAATGTCTCCTGGACCCGAAAGTTTCCTCTAGATCAAGCTCTCTTA
CAGTTGGAGAGCAAATCTGCGCCGTCTTCATACCTTTCTTTGCCGTTGTTGATTTCCTCTT
CTCCACCATGGGACAGTGCTTTGATTGCTGCCGTGTCCCCAATCGATCGTCTCAGACATG
TCAGCACCTGGATCTGGCTCATCTTGCACGTGAATCTCGATTTTCGATCAATGAAGTTGA
AGCTTTGTTTGAGCTCTTCAAGAAATTGAGCTGTTCAATTATTGATGACGGCTTGATTCAC
AAGGAAGAGCTTCGACTGGCCCTGTTCCAAGTTCCATATGGTGAGAACATCTTTCTTGAT
AGGGTTTTCGATTTGTTTGATGAAAAGAAGAATGGGGTTATTGAGTTTGAGGAATTTATC
CATGCATTAAGTGTCTTTCATCCTTATGCACCAATGGAGGAGAAGATCGACTTTGCATTT
AGGCTCTATGATTTGAGACAAACCGGATTTATCGAGCGCGAAGAGGTACAACAAATGGT
GGCTGCTATTTTGATGGAATCTGATTTGATGCTGTCAGATGAACTTCTAACCATGATTATC
GATAAAACATTTGCTGATGCGGACTCGGATGGGGATGGTAAAATTAGCAAGGAGGAGTG
GAAGACTTATGTGCTTAAGCATCCAACCTTGTTAAAGAATATGACTCTTCCTTACTTAAA
GGATGTGACAACAGCATTCCCAAGTTTTATATTCAATACTGAGGTTGAAGAC
aN2 chimeric protein amino acid sequence
SEQ ID NO: 13
MVPVNQCLLDPKVSSRSSSLTVGEQICAVFIPFFAVVDFLFSTMGQCFDCCRVPN
RSSQTCQHLDLAHLARESRFSINEVEALFELFKKLSCSIIDDGLIHKEELRLALFQ
VPYGENIFLDRVFDLFDEKKNGVIEFEEFIHALSVFHPYAPMEEKIDFAFRLYDL
RQTGFIEREEVQQMVAAILMESDLMLSDELLTMIIDKTFADADSDGDGKISKEE
WKTYVLKHPTLLKNMTLPYLKDVTTAFPSFIFNTEVED
aN3 chimeric protein nucleotide sequence
SEQ ID NO: 14
ATGGTCCCGGTTAATCAATGTCTCCTGGACCCGAAAGTTTCCTCTAGATCCAGTTCTTTGA
CTGTTGGGGAGAAAATCTGCGCCGTCTTCATACCTTTAATCGCCATTATTGATATTCTCTT
CTCCACCGTTGGACAGTGCTTTGATTGCTGCCGTGTCCCCAATCGATCGTCTCAGACATGT
CAGCACCTGGATCTGGCTCATCTTGCACGTGAATCTCGATTTTCGATCAATGAAGTTGAA
GCTTTGTTTGAGCTCTTCAAGAAATTGAGCTGTTCAATTATTGATGACGGCTTGATTCACA
AGGAAGAGCTTCGACTGGCCCTGTTCCAAGTTCCATATGGTGAGAACATCTTTCTTGATA
GGGTTTTCGATTTGTTTGATGAAAAGAAGAATGGGGTTATTGAGTTTGAGGAATTTATCC
ATGCATTAAGTGTCTTTCATCCTTATGCACCAATGGAGGAGAAGATCGACTTTGCATTTA
GGCTCTATGATTTGAGACAAACCGGATTTATCGAGCGCGAAGAGGTACAACAAATGGTG
GCTGCTATTTTGATGGAATCTGATTTGATGCTGTCAGATGAACTTCTAACCATGATTATCG
ATAAAACATTTGCTGATGCGGACTCGGATGGGGATGGTAAAATTAGCAAGGAGGAGTGG
AAGACTTATGTGCTTAAGCATCCAACCTTGTTAAAGAATATGACTCTTCCTTACTTAAAG
GATGTGACAACAGCATTCCCAAGTTTTATATTCAATACTGAGGTTGAAGAC
aN3 chimeric protein amino acid sequence
SEQ ID NO: 15
MVPVNQCLLDPKVSSRSSSLTVGEKICAVFIPLIAIIDILFSTVGQCFDCCRVPNRS
SQTCQHLDLAHLARESRFSINEVEALFELFKKLSCSIIDDGLIHKEELRLALFQVP
YGENIFLDRVFDLFDEKKNGVIEFEEFIHALSVFHPYAPMEEKIDFAFRLYDLRQ
TGFIEREEVQQMVAAILMESDLMLSDELLTMIIDKTFADADSDGDGKISKEEWK
TYVLKHPTLLKNMTLPYLKDVTTAFPSFIFNTEVED
bN1 chimeric protein nucleotide sequence
SEQ ID NO: 16
ATGGACTGGCCCAGATTTTCCTCTAGATCCAGTTCTTTGACTGTTGGGGAGAAAA
TCTGCGCCGTCTTCATACCTTTAATCGCCATTATTGATATTCTCTTCTCCACCGTTG
GACAGTGCTTTGATTGCTGCCGTGTCCCCAATCGATCGTCTCAGACATGTCAGCA
CCTGGATCTGGCTCATCTTGCACGTGAATCTCGATTTTCGATCAATGAAGTTGAA
GCTTTGTATGAGCTATTCAAGAAACTGAGCTGTTCCATTATTGATGACGGCTTGA
TTCACAAGGAAGAGCTTAGACTGGCCCTGTTCCAAGCTCCATATGGCGAGAACCT
CTTTCTTGATAGGGTTTTTGATTTGTTCGATGAAAAGAGGAATGGTGTTATTGAGT
TTGAGGAATTCATCCATGCATTGAGTGTCTTTCATCCCTATGCACCGATTGAGGA
GAAGATCGACTTTGCATTTAGGCTCTATGATCTAAGACAAACCGGATTTATTGAG
CGAGAAGAGGTGCAACAAATGGTGGTTGCTATTTTGATGGAATCTGATATGATGC
TGTCCGATGAACTTCTGACAATGATTATTGATAAAACATTTGCTGATGCAGATGC
GGATAAAGATGGTAAAATTAGCAAGGGAGAATGGAAGGTGTATGTGCTTAAGCA
TCCCGCCTTATTGAAGAATATGACTCTTCCTTACCTAAAAGATGTGACGACAGCA
TTTCCAAGTTTTATATTCAACACAGAGGTTGAAGACTGA
bN1 chimeric protein amino acid sequence
SEQ ID NO: 17
MDWPRFSSRSSSLTVGEKICAVFIPLIAIIDILFSTVGQCFDCCRVPNRSSQTCQHL
DLAHLARESRFSINEVEALYELFKKLSCSIIDDGLIHKEELRLALFQAPYGENLFL
DRVFDLFDEKRNGVIEFEEFIHALSVFHPYAPIEEKIDFAFRLYDLRQTGFIEREE
VQQMVVAILMESDMMLSDELLTMIIDKTFADADADKDGKISKGEWKVYVLKH
PALLKNMTLPYLKDVTTAFPSFIFNTEVED
bN2 chimeric protein nucleotide sequence
SEQ ID NO: 18
ATGGACTGGCCCAGATTTTCCTCTAGATCCAGTTCTTTGACTGTTGGGGAGAAAATCTGC
GCCGTCTTCATACCTTTAATCGCCATTATTGATATTCTCTTCTCCACCGTTGGACAATGCT
TCGATTGCCACCGCCGCCGCCGCCGGCGGTTGCCTCAGACATGTCAGCACGCGGATCTGG
CTCGTCTAGCTCGTGAATCTCGATTTTCAATCAACGAAGTTGAAGCTTTGTATGAGCTATT
CAAGAAACTGAGCTGTTCCATTATTGATGACGGCTTGATTCACAAGGAAGAGCTTAGACT
GGCCCTGTTCCAAGCTCCATATGGCGAGAACCTCTTTCTTGATAGGGTTTTTGATTTGTTC
GATGAAAAGAGGAATGGTGTTATTGAGTTTGAGGAATTCATCCATGCATTGAGTGTCTTT
CATCCCTATGCACCGATTGAGGAGAAGATCGACTTTGCATTTAGGCTCTATGATCTAAGA
CAAACCGGATTTATTGAGCGAGAAGAGGTGCAACAAATGGTGGTTGCTATTTTGATGGA
ATCTGATATGATGCTGTCCGATGAACTTCTGACAATGATTATTGATAAAACATTTGCTGA
TGCAGATGCGGATAAAGATGGTAAAATTAGCAAGGGAGAATGGAAGGTGTATGTGCTTA
AGCATCCCGCCTTATTGAAGAATATGACTCTTCCTTACCTAAAAGATGTGACGACAGCAT
TTCCAAGTTTTATATTCAACACAGAGGTTGAAGAC
bN2 chimeric protein amino acid sequence
SEQ ID NO: 19
MDWPRFSSRSSSLTVGEKICAVFIPLIAIIDILFSTVGQCFDCHRRRRRRLPQTCQ
HADLARLARESRFSINEVEALYELFKKLSCSIIDDGLIHKEELRLALFQAPYGENL
FLDRVFDLFDEKRNGVIEFEEFIHALSVFHPYAPIEEKIDFAFRLYDLRQTGFIER
EEVQQMVVAILMESDMMLSDELLTMIIDKTFADADADKDGKISKGEWKVYVLK
HPALLKNMTLPYLKDVTTAFPSFIFNTEVED
bN3 chimeric protein nucleotide sequence
SEQ ID NO: 20
ATGGACTGGCCCAGATTTTCCTCTAGATCAAGCTCTCTTACAGTTGGAGAGCAAATCTGC
GCCGTCTTCATACCTTTCTTTGCCGTTGTTGATTTCCTCTTCTCCACCATGGGACAATGCTT
CGATTGCCACCGCCGCCGCCGCCGGCGGTTGCCTCAGACATGTCAGCACGCGGATCTGGC
TCGTCTAGCTCGTGAATCTCGATTTTCAATCAACGAAGTTGAAGCTTTGTATGAGCTATTC
AAGAAACTGAGCTGTTCCATTATTGATGACGGCTTGATTCACAAGGAAGAGCTTAGACTG
GCCCTGTTCCAAGCTCCATATGGCGAGAACCTCTTTCTTGATAGGGTTTTTGATTTGTTCG
ATGAAAAGAGGAATGGTGTTATTGAGTTTGAGGAATTCATCCATGCATTGAGTGTCTTTC
ATCCCTATGCACCGATTGAGGAGAAGATCGACTTTGCATTTAGGCTCTATGATCTAAGAC
AAACCGGATTTATTGAGCGAGAAGAGGTGCAACAAATGGTGGTTGCTATTTTGATGGAA
TCTGATATGATGCTGTCCGATGAACTTCTGACAATGATTATTGATAAAACATTTGCTGAT
GCAGATGCGGATAAAGATGGTAAAATTAGCAAGGGAGAATGGAAGGTGTATGTGCTTAA
GCATCCCGCCTTATTGAAGAATATGACTCTTCCTTACCTAAAAGATGTGACGACAGCATT
TCCAAGTTTTATATTCAACACAGAGGTTGAAGAC
bN3 chimeric protein amino acid sequence
SEQ ID NO: 21
MDWPRFSSRSSSLTVGEQICAVFIPFFAVVDFLFSTMGQCFDCHRRRRRRLPQTC
QHADLARLARESRFSINEVEALYELFKKLSCSIIDDGLIHKEELRLALFQAPYGEN
LFLDRVFDLFDEKRNGVIEFEEFIHALSVFHPYAPIEEKIDFAFRLYDLRQTGFIE
REEVQQMVVAILMESDMMLSDELLTMIIDKTFADADADKDGKISKGEWKVYVL
KHPALLKNMTLPYLKDVTTAFPSFIFNTEVED
EsCBL10a major element nucleotide sequence
SEQ ID NO: 22
AGATCAAGCTCTCTTACAGTTGGAGAGCAAATCTGCGCCGTCTTCATACCTTTCTTTGCCGTTGTTG
ATTTCCTCTTCTCCACCATG
EsCBL10a major element polypeptide
SEQ ID NO: 23
RSSSLTVGEQICAVFIPFFAVVDFLFSTM
EsCBL10a minor element nucleotide sequence
SEQ ID NO: 24
GGCCAGTGCTTCGACTGCCACAGGAGGAGGAGGAGGAGGCTGCCCCAGACCTGCCAGCAC
GCCGACCTGGCCAGGCTGGCCAGGGAGAGCAGGTTCAGCATCAACGAGGTGGAG
EsCBL10a minor element polypeptide
SEQ ID NO: 25
GQCFDCHRRRRRRLPQTCQHADLARLARESRFSINEVE

What is claimed is:

1. An isolated cDNA molecule comprising a nucleotide sequence selected from the group consisting of:
(a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24;
(b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25;
(c) a nucleotide sequence that is at least 90% identical to the nucleotide sequence of (a) or (b);
(d) a nucleotide sequence that differs from the nucleotide sequence of (a) or (b) due to degeneracy of the genetic code; and
(e) any combination of the nucleotide sequences of (a)-(d).

2. The cDNA molecule of claim 1, wherein the nucleotide sequence is operably linked to a heterologous nucleic acid.

3. The cDNA molecule of claim 2, wherein the heterologous nucleic acid is a promoter.

4. An expression vector comprising the cDNA molecule of claim 1, operably linked to one or more control sequences suitable for directing expression in a host cell.

5. A transgenic plant comprising a cell comprising a chimeric nucleic acid construct comprising the isolated cDNA molecule of claim 1.

6. The transgenic plant of claim 5, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop; alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

7. Seeds from the transgenic plant of claim 5 or a progeny plant thereof, wherein the seeds comprise the chimeric nucleic acid construct.

8. A crop comprising a plurality of plants of claim 6.

9. The isolated cDNA molecule of claim 1, wherein the nucleotide sequence is set forth in SEQ ID NO: 1.

10. The isolated cDNA molecule of claim 1, wherein the nucleotide sequence is set forth in SEQ ID NO: 2.

11. The isolated cDNA molecule of claim 1, wherein the nucleotide sequence is set forth in SEQ ID NO: 4.

12. The isolated cDNA molecule of claim 1, wherein the nucleotide sequence is set forth in SEQ ID NO: 5.

13. A method for increasing salt tolerance in a plant in need thereof, comprising: growing the plant under conditions which allow for the expression of a Eutrema salsugineum CALCINEURIN B-LIKE10 (EsCBL10) gene product from an exogenous expression vector in the plant, the vector comprising a promoter that is functional in a plant cell operably linked to a nucleotide sequence selected from the group consisting of:
(a) the nucleotide sequence set forth in SEQ ID NO: 1, SEQ ID NO: 2, SEQ ID NO: 4, SEQ ID NO: 5, SEQ ID NO: 10, SEQ ID NO: 12, SEQ ID NO: 14, SEQ ID NO: 16, SEQ ID NO: 18, SEQ ID NO: 20, SEQ ID NO: 22, or SEQ ID NO: 24;
(b) a nucleotide sequence that encodes a polypeptide having the amino acid sequence set forth in SEQ ID NO: 3, SEQ ID NO: 6, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25;
(c) a nucleotide sequence that is at least 90% identical to the nucleotide sequence of (a) or (b), and which encodes an EsCBL10 protein;
(d) a nucleotide sequence that differs from the nucleotide sequence of (a) or (b) due to degeneracy of the genetic code; and
(e) any combination of the nucleotide sequences of (a)-(d); and
wherein expression of the EsCBL10 gene product results in the plant having an increased salt tolerance as compared to a control plant grown under similar conditions.

14. The method of claim 13, wherein the vector comprises the nucleotide sequence set forth in SEQ ID NO: 2.

15. The method of claim 13, wherein the vector comprises the nucleotide sequence set forth in SEQ ID NO: 5.

16. The method of claim 13, wherein the increased salt tolerance comprises an improvement in fresh weight, root length, or any combination thereof, as compared to a control plant grown under similar conditions.

17. A transgenic plant produced by the method of claim 13, wherein the plant comprises the exogenous expression vector expressing an EsCBL10 gene product and has increased salt tolerance as compared to a control plant grown under similar conditions.

18. The transgenic plant of claim 17, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

19. Seeds from the transgenic plant of claim 17 or a progeny thereof.

20. A method for increasing salt tolerance in a plant in need thereof, comprising introducing into the plant:
(i) a first vector comprising a nucleotide sequence selected from the group consisting of:
(a) the nucleotide sequence set forth in SEQ ID NO: 1 or SEQ ID NO: 2;
(b) a nucleotide sequence that encodes a polypeptide having the amino acid sequence set forth in SEQ ID NO: 3;
(c) a nucleotide sequence that is at least 90% identical to the nucleotide sequence of (a) or (b), and which encodes an EsCBL10a protein;
(d) a nucleotide sequence that differs from the nucleotide sequence of (a) or (b) due to degeneracy of the genetic code; and
(e) any combination of the nucleotide sequences of (a)-(d); and
(ii) a second vector comprising a nucleotide sequence selected from the group consisting of:
(f) the nucleotide sequence set forth in SEQ ID NO: 4 or SEQ ID NO: 5;
(g) a nucleotide sequence that encodes a polypeptide having the amino acid sequence set forth in SEQ ID NO: 6;
(h) a nucleotide sequence that is at least 90% identical to the nucleotide sequence of (f) or (g), and which encodes an EsCBL10b protein;
(i) a nucleotide sequence that differs from the nucleotide sequence of (f) or (g) due to degeneracy of the genetic code; and
(j) any combination of the nucleotide sequences of (f)-(i);
growing the plant under conditions which allow for the expression of an EsCBL10a and EsCBL10b gene product from the nucleotide sequence;
wherein expression of the EsCBL10a and EsCBL10b gene products results in the plant having an increased salt tolerance as compared to a control plant grown under similar conditions.

21. The method of claim 20, wherein the first vector comprises the nucleotide sequence set forth in SEQ ID NO: 2 and the second vector comprises the nucleotide sequence set forth in SEQ ID NO: 5.

22. The method of claim 20, wherein the increased salt tolerance comprises an improvement in fresh weight, root length, or any combination thereof, as compared to a control plant grown under similar conditions.

23. A transgenic plant produced by the method of claim 20, wherein the plant expresses an EsCBL10a and EsCBL10b gene product and has increased salt tolerance as compared to a control plant grown under similar conditions.

24. The transgenic plant of claim 23, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

25. Seeds from the transgenic plant of claim 23 or a progeny thereof.

26. A method for increasing salt tolerance in a plant in need thereof, comprising introducing into the plant:
(i) a first vector comprising a nucleotide sequence selected from the group consisting of:
(a) the nucleotide sequence set forth in SEQ ID NO: 1 or SEQ ID NO: 2;
(b) a nucleotide sequence that encodes a polypeptide having the amino acid sequence set forth in SEQ ID NO: 3;
(c) a nucleotide sequence that is at least 90% identical to the nucleotide sequence of (a) or (b), and which encodes an EsCBL10a protein;
(d) a nucleotide sequence that differs from the nucleotide sequence of (a) or (b) due to degeneracy of the genetic code; and
(e) any combination of the nucleotide sequences of (a)-(d); and
(ii) a second vector comprising a nucleotide sequence selected from the group consisting of:
(f) the nucleotide sequence set forth in SEQ ID NO: 7 or SEQ ID NO: 8;
(g) a nucleotide sequence that encodes a polypeptide having the amino acid sequence set forth in SEQ ID NO: 9;
(h) a nucleotide sequence that is at least 90% identical to the nucleotide sequence of (f) or (g), and which encodes a EsSOS3 protein;
(i) a nucleotide sequence that differs from the nucleotide sequence of (f) or (g) due to degeneracy of the genetic code; and
(j) any combination of the nucleotide sequences of (f)-(i);
growing the plant under conditions which allow for the expression of an EsCBL10a and EsSOS3 gene product from the nucleotide sequence;
wherein expression of the EsCBL10a and EsSOS3 gene products results in the plant having an increased salt tolerance as compared to a control plant grown under similar conditions.

27. The method of claim 26, wherein the first vector comprises the nucleotide sequence set forth in SEQ ID NO: 2 and the second vector comprises the nucleotide sequence set forth in SEQ ID NO: 8.

28. The method of claim 26, wherein the increased salt tolerance comprises an improvement in fresh weight, root length, or any combination thereof, as compared to a control plant grown under similar conditions.

29. A transgenic plant produced by the method of claim 26, wherein the plant expresses an EsCBL10a and EsSOS3 gene product and has increased salt tolerance as compared to a control plant grown under similar conditions.

30. The transgenic plant of claim 29, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

31. Seeds from the transgenic plant of claim 29 or a progeny thereof.

32. A method for increasing salt tolerance in a plant comprising:
crossing a first plant comprising a first vector comprising a nucleotide sequence selected from the group consisting of:
(a) the nucleotide sequence set forth in SEQ ID NO: 1 or SEQ ID NO: 2;
(b) a nucleotide sequence that encodes a polypeptide having the amino acid sequence set forth in SEQ ID NO: 3;
(c) a nucleotide sequence that is at least 90% identical to the nucleotide sequence of (a) or (b), and which encodes an EsCBL10a protein;
(d) a nucleotide sequence that differs from the nucleotide sequence of (a) or (b) due to degeneracy of the genetic code; and
(e) any combination of the nucleotide sequences of (a)-(d);
with a second plant comprising a second vector comprising a nucleotide sequence selected from the group consisting of:
(f) the nucleotide sequence set forth in SEQ ID NO: 4 or SEQ ID NO: 5;
(g) a nucleotide sequence that encodes a polypeptide having the amino acid sequence set forth in SEQ ID NO: 6;
(h) a nucleotide sequence that is at least 90% identical to the nucleotide sequence of (f) or (g), and which encodes an EsCBL10b protein;
(i) a nucleotide sequence that differs from the nucleotide sequence of (f) or (g) due to degeneracy of the genetic code; and
(j) any combination of the nucleotide sequences of (f)-(i);
to produce a plant expressing EsCBL10a and EsCBL10b gene product and having an increased salt tolerance as compared to a control plant.

33. The method of claim 32, wherein the first vector comprises the nucleotide sequence set forth in SEQ ID NO: 2 and the second vector comprises the nucleotide sequence set forth in SEQ ID NO: 5.

34. The method of claim 32, wherein the increased salt tolerance comprises an improvement in fresh weight, root length, or any combination thereof, as compared to a control plant grown under similar conditions.

35. A transgenic plant produced by the method of claim 32, wherein the plant expresses an EsCBL10a and EsCBL10b gene product and has increased salt tolerance as compared to a control plant.

36. The transgenic plant of claim 35, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

37. Seeds from the transgenic plant of claim 35 or a progeny thereof.

38. A method for increasing salt tolerance in a plant comprising:
crossing a first plant comprising a first vector comprising a nucleotide sequence selected from the group consisting of:
(a) the nucleotide sequence set forth in SEQ ID NO: 1 or SEQ ID NO: 2;
(b) a nucleotide sequence that encodes a polypeptide having the amino acid sequence set forth in SEQ ID NO: 3;
(c) a nucleotide sequence that is at least 90% identical to the nucleotide sequence of (a) or (b), and which encodes an EsCBL10a protein;
(d) a nucleotide sequence that differs from the nucleotide sequence of (a) or (b) due to degeneracy of the genetic code; and
(e) any combination of the nucleotide sequences of (a)-(d);
with a second plant comprising a second vector comprising a nucleotide sequence selected from the group consisting of:
(f) the nucleotide sequence set forth in SEQ ID NO: 7 or SEQ ID NO: 8;
(g) a nucleotide sequence that encodes a polypeptide having the amino acid sequence set forth in SEQ ID NO: 9;
(h) a nucleotide sequence that is at least 90% identical to the nucleotide sequence of (f) or (g), and which encodes a EsSOS3 protein;
(i) a nucleotide sequence that differs from the nucleotide sequence of (f) or (g) due to degeneracy of the genetic code; and
(j) any combination of the nucleotide sequences of (f)-(i);
to produce a plant expressing EsCBL10a and EsSOS3 gene product and having an increased salt tolerance as compared to a control plant.

39. The method of claim 38, wherein the first vector comprises the nucleotide sequence set forth in SEQ ID NO: 2 and the second vector comprises the nucleotide sequence set forth in SEQ ID NO: 8.

40. The method of claim 38, wherein the increased salt tolerance comprises an improvement in fresh weight, root length, or any combination thereof, as compared to a control plant grown under similar conditions.

41. A transgenic plant produced by the method of claim 38, wherein the plant expresses an EsCBL10a and EsSOS3 gene product and has increased salt tolerance as compared to a control plant.

42. The transgenic plant of claim 41, wherein the plant is selected from the group consisting of Arabidopsis thaliana, soybean, wheat, oat, peanut, cotton, corn, rice, amaranth, artichoke, asparagus, broccoli, Brussels sprouts, cabbage, canola, carrot, cauliflower, celery, collard greens, flax, kale, lentil, okra, onion, potato, straw, sugar beet, sugar cane, sunflower, tomato, squash, tea, maize, barley, rye, pea, a forage crop, alfalfa, apple, banana, barley, castor bean, chicory, chrysanthemum, clover, cocoa, coffee, cottonseed, crambe, cranberry, dendrobium, dioscorea, eucalyptus, fescue, gladiolus, liliacea, linseed, millet, muskmelon, mustard, oil palm, papaya, pineapple, ornamental plants, beans, rapeseed, ryegrass, safflower, sesame, sorghum, strawberry, tobacco, turf grass, lettuce, bell pepper, cucurbits, pear, peach, orange, grapefruit, lemon, lime, almond, pecan, walnut, hazel, grapes, kiwi, hops, raspberry, blackberry, gooseberry, coniferous plants, hybrid poplar, moss, algae, and any combinations thereof.

43. Seeds from the transgenic plant of claim 41 or a progeny thereof.

44. The isolated cDNA molecule of claim 1, wherein the nucleotide sequence is set forth in SEQ ID NO: 10.

45. The isolated cDNA molecule of claim 1, wherein the nucleotide sequence is set forth in SEQ ID NO: 12.

46. The isolated cDNA molecule of claim 1, wherein the nucleotide sequence is set forth in SEQ ID NO: 14.

47. The isolated cDNA molecule of claim 1, wherein the nucleotide sequence is set forth in SEQ ID NO: 16.

48. The isolated cDNA molecule of claim 1, wherein the nucleotide sequence is set forth in SEQ ID NO: 18.

49. The isolated cDNA molecule of claim 1, wherein the nucleotide sequence is set forth in SEQ ID NO: 20.

50. The isolated cDNA molecule of claim 1, wherein the nucleotide sequence is set forth in SEQ ID NO: 22.

51. The isolated cDNA molecule of claim 1, wherein the nucleotide sequence is set forth in SEQ ID NO: 24.


SEQUENCE LISTING (269 sequences)

SEQ ID NO: 1 - DNA, 1935 bp, Eutrema salsugineum
aaatacaatacctctcatccagaatttcgaaatcttgtgatgtgatctcgacaccatcttggaagtcggcgaggacgaagaagaggacttagaggaggatggtcccggttaatcaatgtctcctggacccgaaagtttcctctgtaaatttctctcatttcgctttgatttagttttctcctttcttctctctcttggttttggattgaaggtaatgttttgggtcttcctttgttaaaacagagatcaagctctcttacagttggagagcaaatctgcgccgtcttcatacctttctttgccgttgttgatttcctcttctccaccatgggacaatgcttcgattgccaccgccgccgccgccggcggttgcctcagacatgtcagcacgcggatctggctcgtctagctcgtgaatctcgatgtgggttttcttcttttcaccctctctctctctggaccaattctttgtcaatgggttttacaatttctctgttatggagctcaaaagtttacaactttggttcgaatgtacttgtagtttcaatcaacgaagttgaagctttgtatgagctattcaagaaactgagctgttccattattgatgacggcttgattcacaaggtaagtttattttttcttgtctcggatactagtgaatagagatccggttgttgaatttgccataggttgaagcttctatgtgtaaattcttttcttgtgctgatgagtattggttgtgagaatatcgatgttcctggtttgtaggaagagcttagactggccctgttccaagctccatatggcgagaacctctttcttgatagggtgagtagatgtgactatgtcctttggagaagactgattagcttttcgtggtttcttggacttgttgagatgaaagttaatgattatatactcgaagttttgtaggtttttgatttgttcgatgaaaagaggaatggtgttattgagtttgaggaattcatccatgcattgagtgtctttcatccctatgcaccgattgaggagaagatcgactgtaagtttcattgaaacaattatcttaagaaaagggatcattgaaccaaaattttgtgtcagttgaacaattattagtaatatatataacccagggatgagtctcaagcatatgtttttaatctgtggcagttgcatttaggctctatgatctaagacaaaccggatttattgagcgagaagaggtaaatgattgtgcccactgcaagttgcaactgtattttgagctcaataacaagttttaagctgctaattttaggtgcaacaaatggtggttgctattttgatggaatctgatatgatgctgtccgatgaacttctgacaatgattattgataaagtgagtgtaattggaatcctttctcgtagtgatgactcatatttgttaaattcagttagcagatggggttttttttaatcctcttcattgtcttttgctagacatttgctgatgcagatgcggataaagatggtaaaattagcaagggagaatggaaggtgtatgtgcttaagcatcccgccttattgaagaatatgactcttccttacctaaagtaccaatttcttttctctctctatttgttagattaactctctaaatctaagataacctctactttgtgatcattaacttatctgtttgaatttgaagagatgtgacgacagcatttccaagttttatattcaacacagaggttgaagactgaggcagaaccgtgtgtgaccttcttctgcttggttaacgtactaaagccgttatttattttaaatgtttttaaactcaaattagttggtgattattagatgtaagtaaaaaatgtgaggttttgccatcatttgatgcacccatttgtagactgttacattgaaaatgtaatgaagcattcttttttc

SEQ ID NO: 2 - DNA, 780 bp, Eutrema salsugineum
atggtcccggttaatcaatgtctcctggacccgaaagtttcctctagatcaagctctcttacagttggagagcaaatctgcgccgtcttcatacctttctttgccgttgttgatttcctcttctccaccatgggacaatgcttcgattgccaccgccgccgccgccggcggttgcctcagacatgtcagcacgcggatctggctcgtctagctcgtgaatctcgattttcaatcaacgaagttgaagctttgtatgagctattcaagaaactgagctgttccattattgatgacggcttgattcacaaggaagagcttagactggccctgttccaagctccatatggcgagaacctctttcttgatagggtttttgatttgttcgatgaaaagaggaatggtgttattgagtttgaggaattcatccatgcattgagtgtctttcatccctatgcaccgattgaggagaagatcgactttgcatttaggctctatgatctaagacaaaccggatttattgagcgagaagaggtgcaacaaatggtggttgctattttgatggaatctgatatgatgctgtccgatgaacttctgacaatgattattgataaaacatttgctgatgcagatgcggataaagatggtaaaattagcaagggagaatggaaggtgtatgtgcttaagcatcccgccttattgaagaatatgactcttccttacctaaaagatgtgacgacagcatttccaagttttatattcaacacagaggttgaagactga

SEQ ID NO: 3 - protein, 259 aa, Eutrema salsugineum
MVPVNQCLLDPKVSSRSSSLTVGEQICAVFIPFFAVVDFLFSTMGQCFDCHRRRRRRLPQTCQHADLARLARESRFSINEVEALYELFKKLSCSIIDDGLIHKEELRLALFQAPYGENLFLDRVFDLFDEKRNGVIEFEEFIHALSVFHPYAPIEEKIDFAFRLYDLRQTGFIEREEVQQMVVAILMESDMMLSDELLTMIIDKTFADADADKDGKISKGEWKVYVLKHPALLKNMTLPYLKDVTTAFPSFIFNTEVED

SEQ ID NO: 4 - DNA, 2003 bp, Eutrema salsugineum
caagatttggaaaaattatgatgtgattcaaaaccaaatcatttagacgaagaagaagaagaggagttgtgcttcaatgagtcatggactggcccagattttcctctgtaattttcctatttctaatttcttctactctatttttgggttttgtgtaatgttttggatcttcctttctctaaacagagatccagttctttgactgttggggagaaaatctgcgccgtcttcatacctttaatcgccattattgatattctcttctccaccgttggacagtgctttgattgctgccgtgtccccaatcgatcgtctcagacatgtcagcacctggatctggctcatcttgcacgtgaatctcgatgtgggttttcttgttttcactctcttatcaatggttttggaaattatcttgttttgtagttcaaaagttttctaagctttggtttgttttgtttgcttttacgcagtttcgatcaatgaagttgaagctttgtttgagctcttcaagaaattgagctgttcaattattgatgacggcttgattcacaaggtaagtttttttttttgtttttctctcattctttttggaaagtatcttagaatattcatctgtttgacattttaatgtgtgattgctttcaaatcaaaatgaatggtttgctcttttagctgatcattactcatctcataaaagtgtcattgttttttttccctcttatgaaatgattaattatgagaattttgatatattcttgttttgtaggaagagcttcgactggccctgttccaagttccatatggtgagaacatctttcttgatagggtgagtgtgtttcttcagagagacgaaatagcttacttcatggtttcttggacttgttgagatgaaagttatgattatatatatatataaattgatgttatgtaggttttcgatttgtttgatgaaaagaagaatggggttattgagtttgaggaatttatccatgcattaagtgtctttcatccttatgcaccaatggaggagaagatcgactgtaaggatcatggaaaaaaaaactcaatgtcctatatatagcaaagctttgtcatataacccatggatatgagtctcaaacatatgtttctaatctgttggcagttgcatttaggctctatgatttgagacaaaccggatttatcgagcgcgaagaggtaaacattatgtgccttgcccattttgatttcttagttgtctttttaagctaaataaaaagttgaagttgttaaatgttaaggtacaacaaatggtggctgctattttgatggaatctgatttgatgctgtcagatgaacttctaaccatgattatcgataaagtgagtgtatttgaaataatttatcttagtgatgaatcatgttttggtatgtttatcttttctctactttttttttgtttggctagacatttgctgatgcggactcggatggggatggtaaaattagcaaggaggagtggaagacttatgtgcttaagcatccaaccttgttaaagaatatgactcttccttacttaaagtaccaaatctactctctttgttagattaactcagtcattaaaaacaaagcctttacgttatgaccaacacttatctacttgaatttgaagggatgtgacaacagcattcccaagttttatattcaatactgaggttgaagactgatgcagaagcatgtccataaaacaaccggtaatttttttttctgcttggcttacatatttttctatatgttgtatatatatgtaaaccaagcagaaaataaattttgatttacatataagaacggatatctttattgtattttacccaaattggttggtgattattaaatgtgagaaatgttgtgaggttccatcgaaggatttacctatccttctcttgcgcgcattgtggactaatacattggaagtaactaagcttttttgttccaaggggaaagagtggaaaagggaaaaaaaa

SEQ ID NO: 5 - DNA, 756 bp, Eutrema salsugineum
atggactggcccagattttcctctagatccagttctttgactgttggggagaaaatctgcgccgtcttcatacctttaatcgccattattgatattctcttctccaccgttggacagtgctttgattgctgccgtgtccccaatcgatcgtctcagacatgtcagcacctggatctggctcatcttgcacgtgaatctcgattttcgatcaatgaagttgaagctttgtttgagctcttcaagaaattgagctgttcaattattgatgacggcttgattcacaaggaagagcttcgactggccctgttccaagttccatatggtgagaacatctttcttgatagggttttcgatttgtttgatgaaaagaagaatggggttattgagtttgaggaatttatccatgcattaagtgtctttcatccttatgcaccaatggaggagaagatcgactttgcatttaggctctatgatttgagacaaaccggatttatcgagcgcgaagaggtacaacaaatggtggctgctattttgatggaatctgatttgatgctgtcagatgaacttctaaccatgattatcgataaaacatttgctgatgcggactcggatggggatggtaaaattagcaaggaggagtggaagacttatgtgcttaagcatccaaccttgttaaagaatatgactcttccttacttaaaggatgtgacaacagcattcccaagttttatattcaatactgaggttgaagactga

SEQ ID NO: 6 - protein, 251 aa, Eutrema salsugineum
MDWPRFSSRSSSLTVGEKICAVFIPLIAIIDILFSTVGQCFDCCRVPNRSSQTCQHLDLAHLARESRFSINEVEALFELFKKLSCSIIDDGLIHKEELRLALFQVPYGENIFLDRVFDLFDEKKNGVIEFEEFIHALSVFHPYAPMEEKIDFAFRLYDLRQTGFIEREEVQQMVAAILMESDLMLSDELLTMIIDKTFADADSDGDGKISKEEWKTYVLKHPTLLKNMTLPYLKDVTTAFPSFIFNTEVED

SEQ ID NO: 7 - DNA, 1517 bp, Eutrema salsugineum
atgggctgctctccgtcgaagaggaagaaggcaacacgaccgccgggatatgaggatcctaaccttctcgcctccgctacgccatgtcagttttaagttcttgatcttgttaatatgatactcgttagaaaatacacataatagacttcggacaaaactccaatctaattctacttttgatattgggagggacaatagtcacggtagcagaagtggaggctttgtatgaactgttcatgaagctaagcagttcaattatcgacgacggtcttattcataaggtttcattctttctttttcatccaatatactctttaacattatcgaaaacagagtcctctgctttgctttttgagatcttttgtattcatatatatatatatatatatatatatatatattggcttagagtttaaagtgtgattgatgttgtaggaagaatttcagctggctttattcagaaataggaatcggaagaaccttttcgctgatcgggtacgtatatatcaactacctacaggctacaaaaaagttacacatttttaacaaaagaaacatatgtattttgtaaatgacgttttgttttatgatattttatccgcagatatttgatgtatttgatgtgaagcgaaatggagtgatcgagtttggggaatttgtccggtctttaggtgtcttccatccagatgcatctgtccatgaaaaaatcaaatgtaactttttctaaacccctccacattgcttcacattcattctatgtgttaaagtgttctttctatgttaactaacaatgagttttatccatgaatttttgcagttgctttcaagttgtacgatttacggcaaactggattcatcgagcgggaagaagtgagtccattttacggttgttttcttggaacctgcaagattatagtttatgtatcaccataactttgtgtgttgttcttacgtatagctgaaagagatggtaatcgctcttcttcacgaatccgaacttatactttccgaagatatgattgaagtaatggtggataaggtgctccatcttttcacaccattacctatttgtttatttttctatctatcactatagtatattaactcgatgttgaagattctaatcttaatataaggagatggtgttgtattatatgcacaggcttttatcgaagcagatcgcaagaacgatgggagaattgatatagatgaatggaaagatttcgtgtccaagaatccgtcgctcatcaaaaacatgactttgccgtatctaaagtgagttttacattgactacttcaaagatttataaaatgtttgctcaaaggagaacccattcttgagccaaaccgaccggtttgattgatgataagtttctttgtaaaatgtagggacataaagggggcgtttccaagttttgtttcatcttgtgaagacgatgaattggagttgcaaaaactaaatttttaagtaacccaatacagattgcagataaagaagataaaaaaaaagccccaac

SEQ ID NO: 8 - DNA, 663 bp, Eutrema salsugineum
atgggctgctctccgtcgaagaggaagaaggcaacacgaccgccgggatatgaggatcctaaccttctcgcctccgctacgccattcacggtagcagaagtggaggctttgtatgaactgttcatgaagctaagcagttcaattatcgacgacggtcttattcataaggaagaatttcagctggctttattcagaaataggaatcggaagaaccttttcgctgatcggatatttgatgtatttgatgtgaagcgaaatggagtgatcgagtttggggaatttgtccggtctttaggtgtcttccatccagatgcatctgtccatgaaaaaatcaaatttgctttcaagttgtacgatttacggcaaactggattcatcgagcgggaagaactgaaagagatggtaatcgctcttcttcacgaatccgaacttatactttccgaagatatgattgaagtaatggtggataaggcttttatcgaagcagatcgcaagaacgatgggagaattgatatagatgaatggaaagatttcgtgtccaagaatccgtcgctcatcaaaaacatgactttgccgtatctaaaggacataaagggggcgtttccaagttttgtttcatcttgtgaagacgatgaattggagttgcaaaaactaaatttttaa

SEQ ID NO: 9 - protein, 220 aa, Eutrema salsugineum
MGCSPSKRKKATRPPGYEDPNLLASATPFTVAEVEALYELFMKLSSSIIDDGLIHKEEFQLALFRNRNRKNLFADRIFDVFDVKRNGVIEFGEFVRSLGVFHPDASVHEKIKFAFKLYDLRQTGFIEREELKEMVIALLHESELILSEDMIEVMVDKAFIEADRKNDGRIDIDEWKDFVSKNPSLIKNMTLPYLKDIKGAFPSFVSSCEDDELELQKLNF

SEQ ID NO: 10 - DNA, 780 bp, Artificial Sequence
  Description of Artificial Sequence Synthetic polynucleotide
atggtcccggttaatcaatgtctcctggacccgaaagtttcctctagatcaagctctcttacagttggagagcaaatctgcgccgtcttcatacctttctttgccgttgttgatttcctcttctccaccatgggacaatgcttcgattgccaccgccgccgccgccggcggttgcctcagacatgtcagcacgcggatctggctcgtctagctcgtgaatctcgattttcaatcaacgaagttgaagctttgtttgagctcttcaagaaattgagctgttcaattattgatgacggcttgattcacaaggaagagcttcgactggccctgttccaagttccatatggtgagaacatctttcttgatagggttttcgatttgtttgatgaaaagaagaatggggttattgagtttgaggaatttatccatgcattaagtgtctttcatccttatgcaccaatggaggagaagatcgactttgcatttaggctctatgatttgagacaaaccggatttatcgagcgcgaagaggtacaacaaatggtggctgctattttgatggaatctgatttgatgctgtcagatgaacttctaaccatgattatcgataaaacatttgctgatgcggactcggatggggatggtaaaattagcaaggaggagtggaagacttatgtgcttaagcatccaaccttgttaaagaatatgactcttccttacttaaaggatgtgacaacagcattcccaagttttatattcaatactgaggttgaagactga

SEQ ID NO: 11 - protein, 259 aa, Artificial Sequence
  Description of Artificial Sequence Synthetic polypeptide
MVPVNQCLLDPKVSSRSSSLTVGEQICAVFIPFFAVVDFLFSTMGQCFDCHRRRRRRLPQTCQHADLARLARESRFSINEVEALFELFKKLSCSIIDDGLIHKEELRLALFQVPYGENIFLDRVFDLFDEKKNGVIEFEEFIHALSVFHPYAPMEEKIDFAFRLYDLRQTGFIEREEVQQMVAAILMESDLMLSDELLTMIIDKTFADADSDGDGKISKEEWKTYVLKHPTLLKNMTLPYLKDVTTAFPSFIFNTEVED

SEQ ID NO: 12 - DNA, 774 bp, Artificial Sequence
  Description of Artificial Sequence Synthetic polynucleotide
atggtcccggttaatcaatgtctcctggacccgaaagtttcctctagatcaagctctcttacagttggagagcaaatctgcgccgtcttcatacctttctttgccgttgttgatttcctcttctccaccatgggacagtgctttgattgctgccgtgtccccaatcgatcgtctcagacatgtcagcacctggatctggctcatcttgcacgtgaatctcgattttcgatcaatgaagttgaagctttgtttgagctcttcaagaaattgagctgttcaattattgatgacggcttgattcacaaggaagagcttcgactggccctgttccaagttccatatggtgagaacatctttcttgatagggttttcgatttgtttgatgaaaagaagaatggggttattgagtttgaggaatttatccatgcattaagtgtctttcatccttatgcaccaatggaggagaagatcgactttgcatttaggctctatgatttgagacaaaccggatttatcgagcgcgaagaggtacaacaaatggtggctgctattttgatggaatctgatttgatgctgtcagatgaacttctaaccatgattatcgataaaacatttgctgatgcggactcggatggggatggtaaaattagcaaggaggagtggaagacttatgtgcttaagcatccaaccttgttaaagaatatgactcttccttacttaaaggatgtgacaacagcattcccaagttttatattcaatactgaggttgaagac

SEQ ID NO: 13 - protein, 258 aa, Artificial Sequence
  Description of Artificial Sequence Synthetic polypeptide
MVPVNQCLLDPKVSSRSSSLTVGEQICAVFIPFFAVVDFLFSTMGQCFDCCRVPNRSSQTCQHLDLAHLARESRFSINEVEALFELFKKLSCSIIDDGLIHKEELRLALFQVPYGENIFLDRVFDLFDEKKNGVIEFEEFIHALSVFHPYAPMEEKIDFAFRLYDLRQTGFIEREEVQQMVAAILMESDLMLSDELLTMIIDKTFADADSDGDGKISKEEWKTYVLKHPTLLKNMTLPYLKDVTTAFPSFIFNTEVED

SEQ ID NO: 14 - DNA, 774 bp, Artificial Sequence
  Description of Artificial Sequence Synthetic polynucleotide
atggtcccggttaatcaatgtctcctggacccgaaagtttcctctagatccagttctttgactgttggggagaaaatctgcgccgtcttcatacctttaatcgccattattgatattctcttctccaccgttggacagtgctttgattgctgccgtgtccccaatcgatcgtctcagacatgtcagcacctggatctggctcatcttgcacgtgaatctcgattttcgatcaatgaagttgaagctttgtttgagctcttcaagaaattgagctgttcaattattgatgacggcttgattcacaaggaagagcttcgactggccctgttccaagttccatatggtgagaacatctttcttgatagggttttcgatttgtttgatgaaaagaagaatggggttattgagtttgaggaatttatccatgcattaagtgtctttcatccttatgcaccaatggaggagaagatcgactttgcatttaggctctatgatttgagacaaaccggatttatcgagcgcgaagaggtacaacaaatggtggctgctattttgatggaatctgatttgatgctgtcagatgaacttctaaccatgattatcgataaaacatttgctgatgcggactcggatggggatggtaaaattagcaaggaggagtggaagacttatgtgcttaagcatccaaccttgttaaagaatatgactcttccttacttaaaggatgtgacaacagcattcccaagttttatattcaatactgaggttgaagac

SEQ ID NO: 15 - protein, 258 aa, Artificial Sequence
  Description of Artificial Sequence Synthetic polypeptide
MVPVNQCLLDPKVSSRSSSLTVGEKICAVFIPLIAIIDILFSTVGQCFDCCRVPNRSSQTCQHLDLAHLARESRFSINEVEALFELFKKLSCSIIDDGLIHKEELRLALFQVPYGENIFLDRVFDLFDEKKNGVIEFEEFIHALSVFHPYAPMEEKIDFAFRLYDLRQTGFIEREEVQQMVAAILMESDLMLSDELLTMIIDKTFADADSDGDGKISKEEWKTYVLKHPTLLKNMTLPYLKDVTTAFPSFIFNTEVED

SEQ ID NO: 16 - DNA, 756 bp, Artificial Sequence
  Description of Artificial Sequence Synthetic polynucleotide
atggactggcccagattttcctctagatccagttctttgactgttggggagaaaatctgcgccgtcttcatacctttaatcgccattattgatattctcttctccaccgttggacagtgctttgattgctgccgtgtccccaatcgatcgtctcagacatgtcagcacctggatctggctcatcttgcacgtgaatctcgattttcgatcaatgaagttgaagctttgtatgagctattcaagaaactgagctgttccattattgatgacggcttgattcacaaggaagagcttagactggccctgttccaagctccatatggcgagaacctctttcttgatagggtttttgatttgttcgatgaaaagaggaatggtgttattgagtttgaggaattcatccatgcattgagtgtctttcatccctatgcaccgattgaggagaagatcgactttgcatttaggctctatgatctaagacaaaccggatttattgagcgagaagaggtgcaacaaatggtggttgctattttgatggaatctgatatgatgctgtccgatgaacttctgacaatgattattgataaaacatttgctgatgcagatgcggataaagatggtaaaattagcaagggagaatggaaggtgtatgtgcttaagcatcccgccttattgaagaatatgactcttccttacctaaaagatgtgacgacagcatttccaagttttatattcaacacagaggttgaagactga

SEQ ID NO: 17 - protein, 251 aa, Artificial Sequence
  Description of Artificial Sequence Synthetic polypeptide
MDWPRFSSRSSSLTVGEKICAVFIPLIAIIDILFSTVGQCFDCCRVPNRSSQTCQHLDLAHLARESRFSINEVEALYELFKKLSCSIIDDGLIHKEELRLALFQAPYGENLFLDRVFDLFDEKRNGVIEFEEFIHALSVFHPYAPIEEKIDFAFRLYDLRQTGFIEREEVQQMVVAILMESDMMLSDELLTMIIDKTFADADADKDGKISKGEWKVYVLKHPALLKNMTLPYLKDVTTAFPSFIFNTEVED

SEQ ID NO: 18 - DNA, 756 bp, Artificial Sequence
  Description of Artificial Sequence Synthetic polynucleotide
atggactggcccagattttcctctagatccagttctttgactgttggggagaaaatctgcgccgtcttcatacctttaatcgccattattgatattctcttctccaccgttggacaatgcttcgattgccaccgccgccgccgccggcggttgcctcagacatgtcagcacgcggatctggctcgtctagctcgtgaatctcgattttcaatcaacgaagttgaagctttgtatgagctattcaagaaactgagctgttccattattgatgacggcttgattcacaaggaagagcttagactggccctgttccaagctccatatggcgagaacctctttcttgatagggtttttgatttgttcgatgaaaagaggaatggtgttattgagtttgaggaattcatccatgcattgagtgtctttcatccctatgcaccgattgaggagaagatcgactttgcatttaggctctatgatctaagacaaaccggatttattgagcgagaagaggtgcaacaaatggtggttgctattttgatggaatctgatatgatgctgtccgatgaacttctgacaatgattattgataaaacatttgctgatgcagatgcggataaagatggtaaaattagcaagggagaatggaaggtgtatgtgcttaagcatcccgccttattgaagaatatgactcttccttacctaaaagatgtgacgacagcatttccaagttttatattcaacacagaggttgaagac

SEQ ID NO: 19 - protein, 252 aa, Artificial Sequence
  Description of Artificial Sequence Synthetic polypeptide
MDWPRFSSRSSSLTVGEKICAVFIPLIAIIDILFSTVGQCFDCHRRRRRRLPQTCQHADLARLARESRFSINEVEALYELFKKLSCSIIDDGLIHKEELRLALFQAPYGENLFLDRVFDLFDEKRNGVIEFEEFIHALSVFHPYAPIEEKIDFAFRLYDLRQTGFIEREEVQQMVVAILMESDMMLSDELLTMIIDKTFADADADKDGKISKGEWKVYVLKHPALLKNMTLPYLKDVTTAFPSFIFNTEVED

SEQ ID NO: 20 - DNA, 756 bp, Artificial Sequence
  Description of Artificial Sequence Synthetic polynucleotide
atggactggcccagattttcctctagatcaagctctcttacagttggagagcaaatctgcgccgtcttcatacctttctttgccgttgttgatttcctcttctccaccatgggacaatgcttcgattgccaccgccgccgccgccggcggttgcctcagacatgtcagcacgcggatctggctcgtctagctcgtgaatctcgattttcaatcaacgaagttgaagctttgtatgagctattcaagaaactgagctgttccattattgatgacggcttgattcacaaggaagagcttagactggccctgttccaagctccatatggcgagaacctctttcttgatagggtttttgatttgttcgatgaaaagaggaatggtgttattgagtttgaggaattcatccatgcattgagtgtctttcatccctatgcaccgattgaggagaagatcgactttgcatttaggctctatgatctaagacaaaccggatttattgagcgagaagaggtgcaacaaatggtggttgctattttgatggaatctgatatgatgctgtccgatgaacttctgacaatgattattgataaaacatttgctgatgcagatgcggataaagatggtaaaattagcaagggagaatggaaggtgtatgtgcttaagcatcccgccttattgaagaatatgactcttccttacctaaaagatgtgacgacagcatttccaagttttatattcaacacagaggttgaagac

SEQ ID NO: 21 - protein, 252 aa, Artificial Sequence
  Description of Artificial Sequence Synthetic polypeptide
MDWPRFSSRSSSLTVGEQICAVFIPFFAVVDFLFSTMGQCFDCHRRRRRRLPQTCQHADLARLARESRFSINEVEALYELFKKLSCSIIDDGLIHKEELRLALFQAPYGENLFLDRVFDLFDEKRNGVIEFEEFIHALSVFHPYAPIEEKIDFAFRLYDLRQTGFIEREEVQQMVVAILMESDMMLSDELLTMIIDKTFADADADKDGKISKGEWKVYVLKHPALLKNMTLPYLKDVTTAFPSFIFNTEVED

SEQ ID NO: 22 - DNA, 87 bp, Eutrema salsugineum
agatcaagctctcttacagttggagagcaaatctgcgccgtcttcatacctttctttgccgttgttgatttcctcttctccaccatg

SEQ ID NO: 23 - protein, 29 aa, Eutrema salsugineum
RSSSLTVGEQICAVFIPFFAVVDFLFSTM

SEQ ID NO: 24 - DNA, 114 bp, Eutrema salsugineum
ggccagtgcttcgactgccacaggaggaggaggaggaggctgccccagacctgccagcacgccgacctggccaggctggccagggagagcaggttcagcatcaacgaggtggag

SEQ ID NO: 25 - protein, 38 aa, Eutrema salsugineum
GQCFDCHRRRRRRLPQTCQHADLARLARESRFSINEVE

SEQ ID NO: 26 - protein, 8 aa, Eutrema salsugineum
MDWPRFSS

SEQ ID NO: 27 - protein, 6 aa, Arabidopsis thaliana
MEQVSS

SEQ ID NO: 28 - protein, 15 aa, Eutrema salsugineum
MVPVNQCLLDPKVSS

SEQ ID NO: 29 - protein, 82 aa, Eutrema salsugineum
MVPVNQCLLDPKVSSRSSSLTVGEQICAVFIPFFAVVDFLFSTMGQCFDCHRRRRRRLPQTCQHADLARLARESRFSINEVE

SEQ ID NO: 30 - protein, 37 aa, Arabidopsis thaliana
MGCSVSKKKKKNAMRPPGYEDPELLASVTPFTVEEVE

SEQ ID NO: 31 - protein, 74 aa, Eutrema salsugineum
MDWPRFSSRSSSLTVGEKICAVFIPLIAIIDILFSTVGQCFDCCRVPNRSSQTCQHLDLAHLARESRFSINEVE

SEQ ID NO: 32 - protein, 69 aa, Arabidopsis thaliana
MEQVSSRSSSLTVGEQFCAVFIPFFAIIDVLVSSVGQCFDCRSTSPRTCQHADLERLARESQFSVNEVE

SEQ ID NO: 33 - DNA, 24 bp, Artificial Sequence
  Description of Artificial Sequence Synthetic primer
tgagctattcaagaaattgagctg

SEQ ID NO: 34 - DNA, 30 bp, Artificial Sequence
  Description of Artificial Sequence Synthetic primer
gcgctcgagatgggctgctctgtatcgaag

SEQ ID NO: 35 - DNA, 20 bp, Artificial Sequence
  Description of Artificial Sequence Synthetic primer
ggttttccgcagaagtgaag

SEQ ID NO: 36 - DNA, 20 bp, Artificial Sequence
  Description of Artificial Sequence Synthetic primer
agtgattctctcgttcttgt

SEQ ID NO: 37 - DNA, 21 bp, Artificial Sequence
  Description of Artificial Sequence Synthetic primer
tgagcacgctcttcttgcttt

SEQ ID NO: 38 - DNA, 29 bp, Artificial Sequence
  Description of Artificial Sequence Synthetic primer
gcgctcgagatggtcccggttaatcaatg

SEQ ID NO: 39 - DNA, 30 bp, Artificial Sequence
  Description of Artificial Sequence Synthetic primer
gcgctcgagatggactggcccagattttcc

SEQ ID NO: 40 - DNA, 19 bp, Artificial Sequence
  Description of Artificial Sequence Synthetic primer
agaggaagaaggcaacacg

SEQ ID NO: 41 - DNA, 20 bp, Artificial Sequence
  Description of Artificial Sequence Synthetic primer
ttacgatggttcagcagcag

SEQ ID NO: 42 - DNA, 18 bp, Artificial Sequence
  Description of Artificial Sequence Synthetic primer
caagcagcacgatacagt

SEQ ID NO: 43 - DNA, 21 bp, Artificial Sequence
  Description of Artificial Sequence Synthetic primer
tgagcacgctcttcttgcttt

SEQ ID NO: 44 - DNA, 22 bp, Artificial Sequence
  Description of Artificial Sequence Synthetic primer
ggttaatcaatgtctcctggac

SEQ ID NO: 45 - DNA, 20 bp, Artificial Sequence
  Description of Artificial Sequence Synthetic primer
cctctagatccagttctttg

SEQ ID NO: 46 - DNA, 26 bp, Artificial Sequence
  Description of Artificial Sequence Synthetic primer
caagcagcatgaagattaaggtcgtt

SEQ ID NO: 47 - DNA, 31 bp, Artificial Sequence
  Description of Artificial Sequence Synthetic primer
gcgctcgagatggaacaagtttcctctagat

SEQ ID NO: 48 - DNA, 29 bp, Artificial Sequence
  Description of Artificial Sequence Synthetic primer
gcgctcgagatggtcccggttaatcaatg

SEQ ID NO: 49 - DNA, 30 bp, Artificial Sequence; identical to SEQ ID NO: 39
  Description of Artificial Sequence Synthetic primer

SEQ ID NO: 50 - DNA, 30 bp, Artificial Sequence
  Description of Artificial Sequence Synthetic primer
gcgctcgagatgggctgctctccgtcgaag

SEQ ID NO: 51 - DNA, 20 bp, Artificial Sequence
  Description of Artificial Sequence Synthetic primer
ggcttgattcacaaggaaga

SEQ ID NO: 52 - DNA, 25 bp, Artificial Sequence
  Description of Artificial Sequence Synthetic primer
agaagtggaggctttgtatgaactg

SEQ ID NO: 53 - DNA, 20 bp, Artificial Sequence
  Description of Artificial Sequence Synthetic primer
gtcgtacaaccggtattgtg

SEQ ID NO: 54 - DNA, 22 bp, Artificial Sequence
  Description of Artificial Sequence Synthetic primer
ggttaatcaatgtctcctggac

SEQ ID NO: 55 - DNA, 20 bp, Artificial Sequence
  Description of Artificial Sequence Synthetic primer
cctctagatccagttctttg

SEQ ID NO: 56 - DNA, 19 bp, Artificial Sequence
  Description of Artificial Sequence Synthetic primer
agaggaagaaggcaacacg

SEQ ID NO: 57 - DNA, 31 bp, Artificial Sequence; identical to SEQ ID NO: 47
  Description of Artificial Sequence Synthetic primer

SEQ ID NO: 58 - DNA, 29 bp, Artificial Sequence
  Description of Artificial Sequence Synthetic primer
gcgctcgagatggtcccggttaatcaatg

SEQ ID NO: 59 - DNA, 30 bp, Artificial Sequence; identical to SEQ ID NO: 39
  Description of Artificial Sequence Synthetic primer

SEQ ID NO: 60 - DNA, 20 bp, Artificial Sequence
  Description of Artificial Sequence Synthetic primer
ggcttgattcacaaggaaga

SEQ ID NO: 61 - DNA, 23 bp, Artificial Sequence
  Description of Artificial Sequence Synthetic primer
gctttgtatgaactgttcaagaa

SEQ ID NO: 62 - DNA, 20 bp, Artificial Sequence
  Description of Artificial Sequence Synthetic primer
aaacggctaccacatccaag

SEQ ID NO: 63 - DNA, 25 bp, Artificial Sequence
  Description of Artificial Sequence Synthetic primer
actaattttaccgtctttgtcagaa

SEQ ID NO: 64 - DNA, 32 bp, Artificial Sequence
  Description of Artificial Sequence Synthetic primer
ggcgaattcgggaagatacgttttgcaattcc

SEQ ID NO: 65 - DNA, 20 bp, Artificial Sequence
  Description of Artificial Sequence Synthetic primer
atttacgaggtggcaccatc

SEQ ID NO: 66 - DNA, 31 bp, Artificial Sequence
  Description of Artificial Sequence Synthetic primer
aaattgggtagtggatccattaactatcaga

SEQ ID NO: 67 - DNA, 19 bp, Artificial Sequence
  Description of Artificial Sequence Synthetic primer
ccactggcaccgttccaat

SEQ ID NO: 68 - DNA, 31 bp, Artificial Sequence
  Description of Artificial Sequence Synthetic primer
ggcggatcccggtcttcaacctctgtgttga

SEQ ID NO: 69 - DNA, 31 bp, Artificial Sequence
  Description of Artificial Sequence Synthetic primer
ggcggatcccggtcttcaacctcagtattga

SEQ ID NO: 70 - DNA, 19 bp, Artificial Sequence
  Description of Artificial Sequence Synthetic primer
gttcttgcgatctgcttcg

SEQ ID NO: 71 - DNA, 20 bp, Artificial Sequence
  Description of Artificial Sequence Synthetic primer
ccagcagcctttctaacgtc

SEQ ID NO: 72 - DNA, 26 bp, Artificial Sequence
  Description of Artificial Sequence Synthetic primer
cctctagatcacacatcgttcctgaa

SEQ ID NO: 73 - DNA, 19 bp, Artificial Sequence
  Description of Artificial Sequence Synthetic primer
ccactggcaccgttccaat

SEQ ID NO: 74 - DNA, 20 bp, Artificial Sequence
  Description of Artificial Sequence Synthetic primer
tggagaagaggaaatcaaca

SEQ ID NO: 75 - DNA, 19 bp, Artificial Sequence
  Description of Artificial Sequence Synthetic primer
gacgatcgattggggacac

SEQ ID NO: 76 - DNA, 25 bp, Artificial Sequence
  Description of Artificial Sequence Synthetic primer
cttggagatccacatctgctggaat

SEQ ID NO: 77 - DNA, 32 bp, Artificial Sequence
  Description of Artificial Sequence Synthetic primer
ggcggatcccggtcttcaacctcagtgttgaa

SEQ ID NO: 78 - DNA, 31 bp, Artificial Sequence; identical to SEQ ID NO: 68
  Description of Artificial Sequence Synthetic primer

SEQ ID NO: 79 - DNA, 31 bp, Artificial Sequence; identical to SEQ ID NO: 69
  Description of Artificial Sequence Synthetic primer

SEQ ID NO: 80 - DNA, 41 bp, Artificial Sequence
  Description of Artificial Sequence Synthetic primer
ggcgaattcgaaaatttagtttttgcaactccaattcatcg

SEQ ID NO: 81 - DNA, 20 bp, Artificial Sequence
  Description of Artificial Sequence Synthetic primer
tcatagagcctaaatgcaaa

SEQ ID NO: 82 - DNA, 27 bp, Artificial Sequence
  Description of Artificial Sequence Synthetic primer
ccaccattacttcaatcatatcttcgg

SEQ ID NO: 83 - DNA, 20 bp, Artificial Sequence
  Description of Artificial Sequence Synthetic primer
gagctggtctttgaggtttc

SEQ ID NO: 84 - DNA, 20 bp, Artificial Sequence
  Description of Artificial Sequence Synthetic primer
ttcttcaataaggcgggatg

SEQ ID NO: 85 - DNA, 22 bp, Artificial Sequence
  Description of Artificial Sequence Synthetic primer
tgggaatgctgttgtcacatcc

SEQ ID NO: 86 - DNA, 19 bp, Artificial Sequence
  Description of Artificial Sequence Synthetic primer
gttcttgcgatctgcttcg

SEQ ID NO: 87 - DNA, 33 bp, Artificial Sequence
  Description of Artificial Sequence Synthetic primer
gcggcggccgctcagtcttcaacctcagtgttg

SEQ ID NO: 88 - DNA, 31 bp, Artificial Sequence
  Description of Artificial Sequence Synthetic primer
tttgcggccgctcagtcttcaacctctgtgt

SEQ ID NO: 89 - DNA, 33 bp, Artificial Sequence
  Description of Artificial Sequence Synthetic primer
tttgcggccgctcagtcttcaacctcagtattg

SEQ ID NO: 90 - DNA, 20 bp, Artificial Sequence
  Description of Artificial Sequence Synthetic primer
tcatagagcctaaatgcaaa

SEQ ID NO: 91 - DNA, 20 bp, Artificial Sequence
  Description of Artificial Sequence Synthetic primer
ttctcgctcgatgaatccag

SEQ ID NO: 92 - DNA, 20 bp, Artificial Sequence
  Description of Artificial Sequence Synthetic primer
cctccaattgttcctcgtta

SEQ ID NO: 93 - DNA, 30 bp, Artificial Sequence; identical to SEQ ID NO: 34
  Description of Artificial Sequence Synthetic primer

SEQ ID NO: 94 - DNA, 20 bp, Artificial Sequence
  Description of Artificial Sequence Synthetic primer
agtgattctctcgttcttgt

SEQ ID NO: 95 - DNA, 21 bp, Artificial Sequence
  Description of Artificial Sequence Synthetic primer
tgagcacgctcttcttgcttt

SEQ ID NO: 96 - DNA, 29 bp, Artificial Sequence
  Description of Artificial Sequence Synthetic primer
gcgctcgagatggtcccggttaatcaatg

SEQ ID NO: 97 - DNA, 30 bp, Artificial Sequence; identical to SEQ ID NO: 39
  Description of Artificial Sequence Synthetic primer

SEQ ID NO: 98 - DNA, 19 bp, Artificial Sequence
  Description of Artificial Sequence Synthetic primer
agaggaagaaggcaacacg

SEQ ID NO: 99 - DNA, 20 bp, Artificial Sequence
  Description of Artificial Sequence Synthetic primer
ttacgatggttcagcagcag

SEQ ID NO: 100 - DNA, 18 bp, Artificial Sequence
  Description of Artificial Sequence Synthetic primer
caagcagcacgatacagt

SEQ ID NO: 101 - DNA, 21 bp, Artificial Sequence
  Description of Artificial Sequence Synthetic primer
tgagcacgctcttcttgcttt

SEQ ID NO: 102 - DNA, 22 bp, Artificial Sequence
  Description of Artificial Sequence Synthetic primer
ggttaatcaatgtctcctggac

SEQ ID NO: 103 - DNA, 20 bp, Artificial Sequence
  Description of Artificial Sequence Synthetic primer
cctctagatccagttctttg

SEQ ID NO: 104 - DNA, 26 bp, Artificial Sequence
  Description of Artificial Sequence Synthetic primer
caagcagcatgaagattaaggtcgtt

SEQ ID NO: 105 - DNA, 31 bp, Artificial Sequence; identical to SEQ ID NO: 47
  Description of Artificial Sequence Synthetic primer

SEQ ID NO: 106 - DNA, 29 bp, Artificial Sequence
  Description of Artificial Sequence Synthetic primer
gcgctcgagatggtcccggttaatcaatg

SEQ ID NO: 107 - DNA, 30 bp, Artificial Sequence; identical to SEQ ID NO: 39
  Description of Artificial Sequence Synthetic primer

SEQ ID NO: 108 - DNA, 30 bp, Artificial Sequence; identical to SEQ ID NO: 50
  Description of Artificial Sequence Synthetic primer

SEQ ID NO: 109 - DNA, 20 bp, Artificial Sequence
  Description of Artificial Sequence Synthetic primer
ggcttgattcacaaggaaga

SEQ ID NO: 110 - DNA, 25 bp, Artificial Sequence
  Description of Artificial Sequence Synthetic primer
agaagtggaggctttgtatgaactg

SEQ ID NO: 111 - DNA, 20 bp, Artificial Sequence
  Description of Artificial Sequence Synthetic primer
gtcgtacaaccggtattgtg

SEQ ID NO: 112 - DNA, 22 bp, Artificial Sequence
  Description of Artificial Sequence Synthetic primer
ggttaatcaatgtctcctggac

SEQ ID NO: 113 - DNA, 20 bp, Artificial Sequence
  Description of Artificial Sequence Synthetic primer
cctctagatccagttctttg

SEQ ID NO: 114 - DNA, 19 bp, Artificial Sequence
  Description of Artificial Sequence Synthetic primer
agaggaagaaggcaacacg

SEQ ID NO: 115 - DNA, 20 bp, Artificial Sequence
  Description of Artificial Sequence Synthetic primer
ggcttgattcacaaggaaga

SEQ ID NO: 116 - DNA, 23 bp, Artificial Sequence
  Description of Artificial Sequence Synthetic primer
gctttgtatgaactgttcaagaa

SEQ ID NO: 117 - DNA, 20 bp, Artificial Sequence
  Description of Artificial Sequence Synthetic primer
aaacggctaccacatccaag

SEQ ID NO: 118 - DNA, 27 bp, Artificial Sequence
  Description of Artificial Sequence Synthetic primer
gcgccatggaacaagtttcctctagat

SEQ ID NO: 119 - DNA, 32 bp, Artificial Sequence
  Description of Artificial Sequence Synthetic primer
gcgccatggagatgggctgctctgtatcgaag

SEQ ID NO: 120 - DNA, 30 bp, Artificial Sequence
  Description of Artificial Sequence Synthetic primer
cgggatccatatggtcccggttaatcaatg

SEQ ID NO: 121 - DNA, 31 bp, Artificial Sequence
  Description of Artificial Sequence Synthetic primer
cgggatccatatggactggcccagattttcc

SEQ ID NO: 122 - DNA, 31 bp, Artificial Sequence
  Description of Artificial Sequence Synthetic primer
ccggaattcatggtgagaaggcaagaggagg

SEQ ID NO: 123 - DNA, 31 bp, Artificial Sequence
  Description of Artificial Sequence Synthetic primer
ccggaattcatggagaacaaaccaagtgtat

SEQ ID NO: 124 - DNA, 32 bp, Artificial Sequence
  Description of Artificial Sequence Synthetic primer
ccccccgggtatgttgatccccaacaaaaaat

SEQ ID NO: 125 - DNA, 31 bp, Artificial Sequence
  Description of Artificial Sequence Synthetic primer
ccggaattcatggaatctccatatccaaaat

SEQ ID NO: 126 - DNA, 32 bp, Artificial Sequence
  Description of Artificial Sequence Synthetic primer
ccccccgggtatggaggaagaacggcgagttc

SEQ ID NO: 127 - DNA, 31 bp, Artificial Sequence
  Description of Artificial Sequence Synthetic primer
ccggaattcatggtcggagcaaaaccggtgg

SEQ ID NO: 128 - DNA, 31 bp, Artificial Sequence
  Description of Artificial Sequence Synthetic primer
ccggaattcatggaatcacttccccagccgc

SEQ ID NO: 129 - DNA, 32 bp, Artificial Sequence
  Description of Artificial Sequence Synthetic primer
ccccccgggtatggtggtaaggaaggtgggca

SEQ ID NO: 130 - DNA, 31 bp, Artificial Sequence
  Description of Artificial Sequence Synthetic primer
ccggaattcatgagtggaagcagaaggaagg

SEQ ID NO: 131 - DNA, 31 bp, Artificial Sequence
  Description of Artificial Sequence Synthetic primer
ccggaattcatggaaaataagccaagtgttt

SEQ ID NO: 132 - DNA, 31 bp, Artificial Sequence
  Description of Artificial Sequence Synthetic primer
ccggaattcatgccagagatcgagattgccg

SEQ ID NO: 133 - DNA, 32 bp, Artificial Sequence
  Description of Artificial Sequence Synthetic primer
ccccccgggtatggcggagaaaatcacgagag

SEQ ID NO: 134 - DNA, 31 bp, Artificial Sequence
  Description of Artificial Sequence Synthetic primer
ccggaattcatggctcaagtactatctacac

SEQ ID NO: 135 - DNA, 32 bp, Artificial Sequence
  Description of Artificial Sequence Synthetic primer
ccccccgggtatggtagattctgacccggtgg

SEQ ID NO: 136 - DNA, 32 bp, Artificial Sequence
  Description of Artificial Sequence Synthetic primer
ccccccgggtatggagaagaaaggatctgtgt

SEQ ID NO: 137 - DNA, 32 bp, Artificial Sequence
  Description of Artificial Sequence Synthetic primer
ccccccgggtatggaagaatcaaaccgtagta

SEQ ID NO: 138 - DNA, 32 bp, Artificial Sequence
  Description of Artificial Sequence Synthetic primer
ccccccgggtatggtgataaagggaatgcgtg

SEQ ID NO: 139 - DNA, 31 bp, Artificial Sequence
  Description of Artificial Sequence Synthetic primer
ccggaattcatggctcaagccttggctcaac

SEQ ID NO: 140 - DNA, 31 bp, Artificial Sequence
  Description of Artificial Sequence Synthetic primer
ccggaattcatggcggatttgttaagaaaag

SEQ ID NO: 141 - DNA, 32 bp, Artificial Sequence
  Description of Artificial Sequence Synthetic primer
ccccccgggtatggataaaaacggcatagttt

SEQ ID NO: 142 - DNA, 31 bp, Artificial Sequence
  Description of Artificial Sequence Synthetic primer
ccggaattcatgggtttgtttggaacgaaga

SEQ ID NO: 143 - DNA, 32 bp, Artificial Sequence
  Description of Artificial Sequence Synthetic primer
ccccccgggtatgtcttttacaattcctagac

SEQ ID NO: 144 - DNA, 32 bp, Artificial Sequence
  Description of Artificial Sequence Synthetic primer
ccccccgggtatggcttctcgaacaacgcctt

SEQ ID NO: 145 - DNA, 31 bp, Artificial Sequence
  Description of Artificial Sequence Synthetic primer
ccggaattcatgacaaagaaaatgagaagag

SEQ ID NO: 146 - DNA, 32 bp, Artificial Sequence
  Description of Artificial Sequence Synthetic primer
ccccccgggtatgggatccaaacttaaacttt

SEQ ID NO: 147 - DNA, 32 bp, Artificial Sequence
  Description of Artificial Sequence Synthetic primer
ccccccgggtatgaatcggccaaaggttcagc

SEQ ID NO: 148 - DNA, 29 bp, Artificial Sequence
  Description of Artificial Sequence Synthetic primer
gcgctcgagatggtcccggttaatcaatg

SEQ ID NO: 149 - DNA, 35 bp, Artificial Sequence
  Description of Artificial Sequence Synthetic primer
ctcttctccaccatgggacagtgctttgattgctg

SEQ ID NO: 150 - DNA, 30 bp, Artificial Sequence; identical to SEQ ID NO: 39
  Description of Artificial Sequence Synthetic primer

SEQ ID NO: 151 - DNA, 35 bp, Artificial Sequence
  Description of Artificial Sequence Synthetic primer
ctcttctccaccgttggacaatgcttcgattgcca

SEQ ID NO: 152 - DNA, 32 bp, Artificial Sequence; identical to SEQ ID NO: 64
  Description of Artificial Sequence Synthetic primer

SEQ ID NO: 153 - DNA, 31 bp, Artificial Sequence; identical to SEQ ID NO: 66
  Description of Artificial Sequence Synthetic primer

SEQ ID NO: 154 - DNA, 19 bp, Artificial Sequence
  Description of Artificial Sequence Synthetic primer
ccactggcaccgttccaat

SEQ ID NO: 155 - DNA, 31 bp, Artificial Sequence; identical to SEQ ID NO: 68
  Description of Artificial Sequence Synthetic primer

SEQ ID NO: 156 - DNA, 31 bp, Artificial Sequence; identical to SEQ ID NO: 69
  Description of Artificial Sequence Synthetic primer

SEQ ID NO: 157 - DNA, 19 bp, Artificial Sequence
  Description of Artificial Sequence Synthetic primer
gttcttgcgatctgcttcg

SEQ ID NO: 158 - DNA, 20 bp, Artificial Sequence
  Description of Artificial Sequence Synthetic primer
ccagcagcctttctaacgtc

SEQ ID NO: 159 - DNA, 26 bp, Artificial Sequence
  Description of Artificial Sequence Synthetic primer
cctctagatcacacatcgttcctgaa

SEQ ID NO: 160 - DNA, 19 bp, Artificial Sequence
  Description of Artificial Sequence Synthetic primer
ccactggcaccgttccaat

SEQ ID NO: 161 - DNA, 20 bp, Artificial Sequence
  Description of Artificial Sequence Synthetic primer
tggagaagaggaaatcaaca

SEQ ID NO: 162 - DNA, 19 bp, Artificial Sequence
  Description of Artificial Sequence Synthetic primer
gacgatcgattggggacac

SEQ ID NO: 163 - DNA, 25 bp, Artificial Sequence
  Description of Artificial Sequence Synthetic primer
cttggagatccacatctgctggaat

SEQ ID NO: 164 - DNA, 32 bp, Artificial Sequence; identical to SEQ ID NO: 77
  Description of Artificial Sequence Synthetic primer

SEQ ID NO: 165 - DNA, 31 bp, Artificial Sequence; identical to SEQ ID NO: 68
  Description of Artificial Sequence Synthetic primer

SEQ ID NO: 166 - DNA, 31 bp, Artificial Sequence; identical to SEQ ID NO: 69
  Description of Artificial Sequence Synthetic primer

SEQ ID NO: 167 - DNA, 41 bp, Artificial Sequence; identical to SEQ ID NO: 80
  Description of Artificial Sequence Synthetic primer

SEQ ID NO: 168 - DNA, 20 bp, Artificial Sequence
  Description of Artificial Sequence Synthetic primer
tcatagagcctaaatgcaaa

SEQ ID NO: 169 - DNA, 27 bp, Artificial Sequence
  Description of Artificial Sequence Synthetic primer
ccaccattacttcaatcatatcttcgg

SEQ ID NO: 170 - DNA, 20 bp, Artificial Sequence
  Description of Artificial Sequence Synthetic primer
gagctggtctttgaggtttc

SEQ ID NO: 171 - DNA, 20 bp, Artificial Sequence
  Description of Artificial Sequence Synthetic primer
ttcttcaataaggcgggatg

SEQ ID NO: 172 - DNA, 22 bp, Artificial Sequence
  Description of Artificial Sequence Synthetic primer
tgggaatgctgttgtcacatcc

SEQ ID NO: 173 - DNA, 19 bp, Artificial Sequence
  Description of Artificial Sequence Synthetic primer
gttcttgcgatctgcttcg

SEQ ID NO: 174 - DNA, 20 bp, Artificial Sequence
  Description of Artificial Sequence Synthetic primer
tcatagagcctaaatgcaaa

SEQ ID NO: 175 - DNA, 20 bp, Artificial Sequence
  Description of Artificial Sequence Synthetic primer
ttctcgctcgatgaatccag

SEQ ID NO: 176 - DNA, 20 bp, Artificial Sequence
  Description of Artificial Sequence Synthetic primer
cctccaattgttcctcgtta

SEQ ID NO: 177 - DNA, 32 bp, Artificial Sequence; identical to SEQ ID NO: 77
  Description of Artificial Sequence Synthetic primer

SEQ ID NO: 178 - DNA, 32 bp, Artificial Sequence; identical to SEQ ID NO: 64
  Description of Artificial Sequence Synthetic primer

SEQ ID NO: 179 - DNA, 28 bp, Artificial Sequence
  Description of Artificial Sequence Synthetic primer
ttctcgagtcagtcttcaacctctgtgt

SEQ ID NO: 180 - DNA, 30 bp, Artificial Sequence
  Description of Artificial Sequence Synthetic primer
ttctcgagtcagtcttcaacctcagtattg

SEQ ID NO: 181 - DNA, 31 bp, Artificial Sequence
  Description of Artificial Sequence Synthetic primer
cgcggatccctaagttactatctcttgctcc

SEQ ID NO: 182 - DNA, 31 bp, Artificial Sequence
  Description of Artificial Sequence Synthetic primer
cgcggatccctatgatggttcttgctctcct

SEQ ID NO: 183 - DNA, 31 bp, Artificial Sequence
  Description of Artificial Sequence Synthetic primer
ccgctcgagtcactttgctgtttctttctta

SEQ ID NO: 184 - DNA, 31 bp, Artificial Sequence
  Description of Artificial Sequence Synthetic primer
cgcggatcctcaattgtgccatgagagcaca

SEQ ID NO: 185 - DNA, 31 bp, Artificial Sequence
  Description of Artificial Sequence Synthetic primer
ccgctcgagtcaacaatcctcggaagaagtg

SEQ ID NO: 186 - DNA, 31 bp, Artificial Sequence
  Description of Artificial Sequence Synthetic primer
cgcggatcctcaagcaggtgtagaggtccag

SEQ ID NO: 187 - DNA, 31 bp, Artificial Sequence
  Description of Artificial Sequence Synthetic primer
cgcggatccttacatgatgtcattgtgccat

SEQ ID NO: 188 - DNA, 31 bp, Artificial Sequence
  Description of Artificial Sequence Synthetic primer
ccgctcgagtcaacgtcttttactcttggcc

SEQ ID NO: 189 - DNA, 31 bp, Artificial Sequence
  Description of Artificial Sequence Synthetic primer
cgcggatccttattgcttttgttcttcagcg

SEQ ID NO: 190 - DNA, 31 bp, Artificial Sequence
  Description of Artificial Sequence Synthetic primer
cgcggatcctcaaaacttcaatggttcttcc

SEQ ID NO: 191 - DNA, 31 bp, Artificial Sequence
  Description of Artificial Sequence Synthetic primer
cgcggatccttaaatagccgcgtttgttgac

SEQ ID NO: 192 - DNA, 31 bp, Artificial Sequence
  Description of Artificial Sequence Synthetic primer
ccgctcgagctattcagtgtcagacggcaag

SEQ ID NO: 193 - DNA, 31 bp, Artificial Sequence
  Description of Artificial Sequence Synthetic primer
cgcggatcctcactgttcaatttcaggtggc

SEQ ID NO: 194 - DNA, 31 bp, Artificial Sequence
  Description of Artificial Sequence Synthetic primer
ccgctcgagctacgacgtcgtatgtacttga

SEQ ID NO: 195 - DNA, 31 bp, Artificial Sequence
  Description of Artificial Sequence Synthetic primer
ccgctcgagtcagtgccaagctaatacaaag

SEQ ID NO: 196 - DNA, 31 bp, Artificial Sequence
  Description of Artificial Sequence Synthetic primer
ccgctcgagtcatgaaacattatttattttg

SEQ ID NO: 197 - DNA, 31 bp, Artificial Sequence
  Description of Artificial Sequence Synthetic primer
ccgctcgagctacgctaaaagctcctgtact

SEQ ID NO: 198 - DNA, 31 bp, Artificial Sequence
  Description of Artificial Sequence Synthetic primer
cgcggatccctattcagtatcagatggcaaa

SEQ ID NO: 199 - DNA, 31 bp, Artificial Sequence
  Description of Artificial Sequence Synthetic primer
cgcggatccctaatcagtatcagaaagtaaa

SEQ ID NO: 200 - DNA, 31 bp, Artificial Sequence
  Description of Artificial Sequence Synthetic primer
ccgctcgagttaatgtatcacttcaatcttc

SEQ ID NO: 201 - DNA, 31 bp, Artificial Sequence
  Description of Artificial Sequence Synthetic primer
cgcggatccttagcttacttccgcggtaagt

SEQ ID NO: 202 - DNA, 31 bp, Artificial Sequence
  Description of Artificial Sequence Synthetic primer
ccgctcgagttacggtttgtcaggaacttta

SEQ ID NO: 203 - DNA, 31 bp, Artificial Sequence
  Description of Artificial Sequence Synthetic primer
ccgctcgagttatgtcgactgttttgcaatt

SEQ ID NO: 204 - DNA, 31 bp, Artificial Sequence
  Description of Artificial Sequence Synthetic primer
cgcggatcctcaaaacgtgattgttctgaga

SEQ ID NO: 205 - DNA, 31 bp, Artificial Sequence
  Description of Artificial Sequence Synthetic primer
ccggtcgacttagcagtcactaccagaattt

SEQ ID NO: 206 - DNA, 31 bp, Artificial Sequence
  Description of Artificial Sequence Synthetic primer
ccgctcgagttatttgcttagaccagagctc

SEQ ID NO: 207 - DNA, 35 bp, Artificial Sequence
  Description of Artificial Sequence Synthetic primer
atcaaagcactgtcccatggtggagaagaggaaat

SEQ ID NO: 208 - DNA, 33 bp, Artificial Sequence; identical to SEQ ID NO: 89
  Description of Artificial Sequence Synthetic primer

SEQ ID NO: 209 - DNA, 31 bp, Artificial Sequence; identical to SEQ ID NO: 69
  Description of Artificial Sequence Synthetic primer

SEQ ID NO: 210 - DNA, 35 bp, Artificial Sequence
  Description of Artificial Sequence Synthetic primer
atcgaagcattgtccaacggtggagaagagaatat

SEQ ID NO: 211 - DNA, 31 bp, Artificial Sequence; identical to SEQ ID NO: 88
  Description of Artificial Sequence Synthetic primer

SEQ ID NO: 212 - DNA, 31 bp, Artificial Sequence; identical to SEQ ID NO: 68
  Description of Artificial Sequence Synthetic primer

SEQ ID NO: 213 - DNA, 21 bp, Artificial Sequence
  Description of Artificial Sequence Synthetic oligonucleotide
tacattgattaacggggacca

SEQ ID NO: 214 - DNA, 21 bp, Artificial Sequence
  Description of Artificial Sequence Synthetic oligonucleotide
taccatccccatcccagtccg

SEQ ID NO: 215 - DNA, 21 bp, Artificial Sequence
  Description of Artificial Sequence Synthetic oligonucleotide
ttcacgtgcaagatgagccac

SEQ ID NO: 216 - DNA, 21 bp, Artificial Sequence
  Description of Artificial Sequence Synthetic oligonucleotide
tcatcaaaatagcagccacca

SEQ ID NO: 217 - DNA, 21 bp, Artificial Sequence
  Description of Artificial Sequence Synthetic oligonucleotide
tagaagttcatctgacagcac

SEQ ID NO: 218 - DNA, 40 bp, Artificial Sequence
  Description of Artificial Sequence Synthetic primer
gattcacgagctagacgcgccattctctcttttgtattcc

SEQ ID NO: 219 - DNA, 40 bp, Artificial Sequence
  Description of Artificial Sequence Synthetic primer
gaatggcgcgtctagctcgtgaatcaaagagaatcaatga

SEQ ID NO: 220 - DNA, 40 bp, Artificial Sequence
  Description of Artificial Sequence Synthetic primer
gaatagcgcgtctaggtcgtgattcacaggtcgtgatatg

SEQ ID NO: 221 - DNA, 40 bp, Artificial Sequence
  Description of Artificial Sequence Synthetic primer
gaatcacgacctagacgcgctattctacatatatattcct

SEQ ID NO: 222 - DNA, 40 bp, Artificial Sequence
  Description of Artificial Sequence Synthetic primer
gataccatccccatcccagtccgtctctcttttgtattcc

SEQ ID NO: 223 - DNA, 40 bp, Artificial Sequence
  Description of Artificial Sequence Synthetic primer
gacggactgggatggggatggtatcaaagagaatcaatga

SEQ ID NO: 224 - DNA, 40 bp, Artificial Sequence
  Description of Artificial Sequence Synthetic primer
gacgaactgggatggcgatggtttcacaggtcgtgatatg

SEQ ID NO: 225 - DNA, 40 bp, Artificial Sequence
  Description of Artificial Sequence Synthetic primer
gaaaccatcgccatcccagttcgtctacatatatattcct

SEQ ID NO: 226 - DNA, 40 bp, Artificial Sequence
  Description of Artificial Sequence Synthetic primer
gattcacgtgcaagatgagccactctctcttttgtattcc

SEQ ID NO: 227 - DNA, 40 bp, Artificial Sequence
  Description of Artificial Sequence Synthetic primer
gagtggctcatcttgcacgtgaatcaaagagaatcaatga

SEQ ID NO: 228 - DNA, 40 bp, Artificial Sequence
  Description of Artificial Sequence Synthetic primer
gagtagctcatcttggacgtgattcacaggtcgtgatatg

SEQ ID NO: 229 - DNA, 40 bp, Artificial Sequence
  Description of Artificial Sequence Synthetic primer
gaatcacgtccaagatgagctactctacatatatattcct

SEQ ID NO: 230 - DNA, 40 bp, Artificial Sequence
  Description of Artificial Sequence Synthetic primer
gatcatcaaaatagcagccaccatctctcttttgtattcc

SEQ ID NO: 231 - DNA, 40 bp, Artificial Sequence
  Description of Artificial Sequence Synthetic primer
gatggtggctgctattttgatgatcaaagagaatcaatga

SEQ ID NO: 232 - DNA, 40 bp, Artificial Sequence
  Description of Artificial Sequence Synthetic primer
gatgatggctgctatattgatgttcacaggtcgtgatatg

SEQ ID NO: 233 - DNA, 40 bp, Artificial Sequence
  Description of Artificial Sequence Synthetic primer
gaacatcaatatagcagccatcatctacatatatattcct

SEQ ID NO: 234 - DNA, 40 bp, Artificial Sequence
  Description of Artificial Sequence Synthetic primer
gatagaagttcatctgacagcactctctcttttgtattcc

SEQ ID NO: 235 - DNA, 40 bp, Artificial Sequence
  Description of Artificial Sequence Synthetic primer
gagtgctgtcagatgaacttctatcaaagagaatcaatga

SEQ ID NO: 236 - DNA, 40 bp, Artificial Sequence
  Description of Artificial Sequence Synthetic primer
gagtactgtcagatgtacttctttcacaggtcgtgatatg

SEQ ID NO: 237 - DNA, 40 bp, Artificial Sequence
  Description of Artificial Sequence Synthetic primer
gaaagaagtacatctgacagtactctacatatatattcct

SEQ ID NO: 238 - DNA, 21 bp, Artificial Sequence
  Description of Artificial Sequence Synthetic oligonucleotide
tggtcccggttaatcaatgtc

SEQ ID NO: 239 - DNA, 21 bp, Artificial Sequence
  Description of Artificial Sequence Synthetic oligonucleotide
atgctgtccgatgaacttctg

SEQ ID NO: 240 - DNA, 21 bp, Artificial Sequence
  Description of Artificial Sequence Synthetic oligonucleotide
tggtggttgctattttgatgg

SEQ ID NO: 241 - DNA, 21 bp, Artificial Sequence
  Description of Artificial Sequence Synthetic oligonucleotide
atgctgtcagatgaacttcta

SEQ ID NO: 242 - DNA, 21 bp, Artificial Sequence
  Description of Artificial Sequence Synthetic oligonucleotide
tggtggctgctattttgatgg

SEQ ID NO: 243 - DNA, 21 bp, Artificial Sequence
  Description of Artificial Sequence Synthetic oligonucleotide
ctggctcatcttgcacgtgaa

SEQ ID NO: 244 - DNA, 21 bp, Artificial Sequence
  Description of Artificial Sequence Synthetic oligonucleotide
cggactcggatggggatggta

SEQ ID NO: 245 - DNA, 21 bp, Artificial Sequence
  Description of Artificial Sequence Synthetic oligonucleotide
tacattgattaacggggacca

SEQ ID NO: 246 - DNA, 21 bp, Artificial Sequence
  Description of Artificial Sequence Synthetic oligonucleotide
atgtaactaattgcccctggt

SEQ ID NO: 247 - DNA, 21 bp, Artificial Sequence
  Description of Artificial Sequence Synthetic oligonucleotide
ctgtaactaattggccctggt

SEQ ID NO: 248 - DNA, 21 bp, Artificial Sequence
  Description of Artificial Sequence Synthetic oligonucleotide
aggtaaccacgtattcctact

SEQ ID NO: 249 - DNA, 21 bp, Artificial Sequence
  Description of Artificial Sequence Synthetic oligonucleotide
taccatccccatcccagtccg

SEQ ID NO: 250 - DNA, 21 bp, Artificial Sequence
  Description of Artificial Sequence Synthetic oligonucleotide
atggtaggggtagggtcaggc

SEQ ID NO: 251 - DNA, 21 bp, Artificial Sequence
  Description of Artificial Sequence Synthetic oligonucleotide
atggtagaaataggcgtagac

SEQ ID NO: 252 - DNA, 21 bp, Artificial Sequence
  Description of Artificial Sequence Synthetic oligonucleotide
atggtaggggtaggctcaggc

SEQ ID NO: 253 - DNA, 21 bp, Artificial Sequence
  Description of Artificial Sequence Synthetic oligonucleotide
ttcacgtgcaagatgagccac

SEQ ID NO: 254 - DNA, 21 bp, Artificial Sequence
  Description of Artificial Sequence Synthetic oligonucleotide
aagtgcacgttctactcggtg

SEQ ID NO: 255 - DNA, 21 bp, Artificial Sequence
  Description of Artificial Sequence Synthetic oligonucleotide
aagtgctcgatctgctcggtc

SEQ ID NO: 256 - DNA, 21 bp, Artificial Sequence
  Description of Artificial Sequence Synthetic oligonucleotide
aagtgcacgttctactcggtc

SEQ ID NO: 257 - DNA, 21 bp, Artificial Sequence
  Description of Artificial Sequence Synthetic oligonucleotide
tcatcaaaatagcagccacca

SEQ ID NO: 258 - DNA, 21 bp, Artificial Sequence
  Description of Artificial Sequence Synthetic oligonucleotide
agtagttttatcgtcggtggt

SEQ ID NO: 259 - DNA, 21 bp, Artificial Sequence
  Description of Artificial Sequence Synthetic oligonucleotide
ggtagttttatcgttggtggt

SEQ ID NO: 260 - DNA, 21 bp, Artificial Sequence
  Description of Artificial Sequence Synthetic oligonucleotide
ggtagttttatcgtcggtggt

SEQ ID NO: 261 - DNA, 21 bp, Artificial Sequence
  Description of Artificial Sequence Synthetic oligonucleotide
tagaagttcatctgacagcac

SEQ ID NO: 262 - DNA, 21 bp, Artificial Sequence
  Description of Artificial Sequence Synthetic oligonucleotide
atcttcaagtagactgtcgtg

SEQ ID NO: 263 - DNA, 21 bp, Artificial Sequence
  Description of Artificial Sequence Synthetic oligonucleotide
gtcttcaagtagcctgtcgta

SEQ ID NO: 264 - DNA, 21 bp, Artificial Sequence
  Description of Artificial Sequence Synthetic oligonucleotide
atcttcaagtagactgtcgta

SEQ ID NO: 265 - protein, 246 aa, Arabidopsis thaliana
MEQVSSRSSSLTVGEQFCAVFIPFFAIIDVLVSSVGQCFDCRSTSPRTCQHADLERLARESQFSVNEVEALYELFKKLSCSIIDDGLIHKEELRLALFQAPYGENLFLDRVFDLFDEKKNGVIEFEEFIHALSVFHPYASIQEKTDFAFRLYDLRQTGFIEREEVQQMVSAILLESDMMLSDELLTMIIDKTFADADSDKDGKISKDEWNVYVHKHPSLLKNMTLPYLKDVTTAFPSFIFNTEVED

SEQ ID NO: 266 - protein, 259 aa, Eutrema salsugineum; identical to SEQ ID NO: 3

SEQ ID NO: 267 - protein, 251 aa, Eutrema salsugineum; identical to SEQ ID NO: 6

SEQ ID NO: 268 - protein, 222 aa, Arabidopsis thaliana
MGCSVSKKKKKNAMRPPGYEDPELLASVTPFTVEEVEALYELFKKLSSSIIDDGLIHKEEFQLALFRNRNRRNLFADRIFDVFDVKRNGVIEFGEFVRSLGVFHPSAPVHEKVKFAFKLYDLRQTGFIEREELKEMVVALLHESELVLSEDMIEVMVDKAFVQADRKNDGKIDIDEWKDFVSLNPSLIKNMTLPYLKDINRTFPSFVSSCEEEEMELQNVSS

SEQ ID NO: 269 - protein, 220 aa, Eutrema salsugineum; identical to SEQ ID NO: 9